(19) United States
(12) Patent Application Publication
(10) Pub. No.: US 2026/0265731 A1
(43) Pub. Date: Sep. 10, 2026

(54) MULTI-SITE EDITING IN LIVING CELLS

(71) Applicants: The Broad Institute, Inc., Cambridge, MA (US); PRESIDENT AND FELLOWS OF HARVARD COLLEGE, Cambridge, MA (US)

(72) Inventors: Verena VOLF, Cambridge, MA (US); George CHURCH, Cambridge, MA (US); Fei CHEN, Cambridge, MA (US)

(73) Assignees: The Broad Institute, Inc., Cambridge, MA (US); PRESIDENT AND FELLOWS OF HARVARD COLLEGE, Cambridge, MA (US)

(21) Appl. No.: 19/665,561

(22) Filed: May 1, 2026

Related U.S. Application Data
(63) Continuation of application No. PCT/US2024/054084, filed on Nov. 1, 2024.
(60) Provisional application No. 63/547,290, filed on Nov. 3, 2023.

(51) Int. Cl. C12N 15/10; C12N 9/22; C12N 9/78; C12N 15/11

(52) U.S. Cl. CPC C12N 15/1086; C12N 9/222; C12N 9/78; C12N 15/1082; C12N 15/111; C12N 2310/20; C12Y 305/04004; C12Y 305/04005

(57) ABSTRACT

Methods and systems described herein enable robust and facile multi-site base editing through pooled editing, suitable for one-step encoding of information. gRNAs show robust editing in pools and demonstrate simultaneous multi-site editing across >100 genomic sites. Additionally, individual stem cells with more than two dozen edits can be obtained with minimal screening.

### STATEMENT REGARDING FEDERALLY SPONSORED RESEARCH

This invention was made with government support under Grant No. OD024583 awarded by the National Institutes of Health. The government has certain rights in the invention.

### TECHNICAL FIELD

The subject matter disclosed herein is generally directed to high-throughput genome-editing techniques including single cell enrichment.

### BACKGROUND

Gene editing, especially through the CRISPR-Cas9 system, has profoundly impacted biotechnology by enabling precise modifications to DNA sequences in a variety of organisms. This technology has accelerated research, with applications spanning medicine, agriculture, and industrial biotechnology. There is an increasing need to be able to make and select for multiple edits in parallel. For example, multiple genomic edits enable the manipulation of several genes simultaneously, which is crucial for engineering complex traits that are governed by multiple genetic factors. This is particularly useful in agriculture for developing crops with multiple desirable traits like increased yield, disease resistance, and drought tolerance. Parallel edits significantly speed up the research and development process by allowing researchers to test the effects of multiple genetic modifications in a single experiment rather than sequentially. This acceleration can lead to faster discoveries and quicker translation of research findings into complex biological systems and diseases. In medicine, the ability to edit multiple loci in parallel could be pivotal for developing therapies caused by mutations in multiple genes or pathways. Further, adoptive cell-based therapies increase the need to make multiple edits to multiple cellular functions in order to develop safe and effective cell-based therapeutics. In industrial biotechnology, the ability to perform multiple edits in parallel enables the creation of cellular factories tailored to produce a variety of chemicals, materials, or biofuels efficiently. In recent years, DNA has gained increased interest as a repurposed medium for encoding data without any cellular function. Encoding information in the DNA of living organisms also requires the ability to make multiple parallel edits, especially where cryptographic encoding is utilized. Thus, the ability to make multiple genetic edits in parallel can greatly increase the efficiency and security with which information is encoded in DNA. Accordingly, there is a need in the art for methods and systems that increase the efficiency with which multiple edits can be made, screened, and enriched for.

Citation or identification of any document in this application is not an admission that such a document is available as prior art to the present invention.

### SUMMARY

In some aspects, the techniques described herein relate to a method of enriching for multi-site edited cells, including (a) delivering to a population of cells, a set of guide molecules and one or more programmable nucleases, wherein the set of guide molecules directs the one or more programmable nucleases to introduce a set of edits to a plurality of genomic loci to generate an edited population of cells; (b) screening the population of cells for one or more enrichment markers; and (c) isolating one or more cells from the population of cells that include the one or more enrichment markers.

In example embodiments, the one or more enrichment markers include an editing efficiency, an exogenous reporter, or a combination thereof. In example embodiments, editing efficiency is determined by sequencing one or more genomic loci having a low overall editing efficiency in the plurality of genomic loci, wherein an edit above an editing efficiency threshold at the one or more genomic loci is an indicator that a cell includes all edits in the set of edits.

In example embodiments, the one or more genomic loci having a low overall editing efficiency are associated with a functional phenotype or an encoded reporter gene, wherein the functional phenotype or encoded reported gene are expressed if the one or more genomic loci having the low overall editing efficiency are successfully edited. In example embodiments, the functional phenotype is antibiotic resistance. In example embodiments, the editing efficiency threshold is at least 0.1%, 0.2%, 0.5%, or 1%.

In example embodiments, the exogenous reporter is co-delivered to the population of cells. In example embodiments, the exogenous reporter is a fluorescent protein. In example embodiments, one or more cells that express the exogenous reporter above an expression threshold are isolated. In example embodiments, the expression threshold is in a top 20%, 15%, 10%, 5%, or 1% of the edited population of cells.

In example embodiments, the method further includes, before the delivering step, preparing a screening pool of guide molecule vectors including the set of guide molecules, wherein the set of guide molecules target a plurality of target sites in a cell genome and are configured to introduce the set of edits. In example embodiments, the screening step is conducted in replicate and selecting the one or more cells includes selecting one or more cells with a correlated enrichment.

In example embodiments, the one or more programmable nucleases is a base editor including a nucleotide deaminase. In example embodiments, the nucleotide deaminase is a cytidine deaminase or an adenosine deaminase. In example embodiments, the one or more programmable nucleases is a prime editor. In example embodiments, the one or more programmable nucleases is a Cas or OMEGA nuclease.

In some aspects, the techniques described herein relate to an engineered cell including edits at a plurality of genomic loci that are obtained by any of the methods described herein. In example embodiments, the engineered cell is a primary cell or a stem cell. In example embodiments, the edits at the plurality of genomic loci are cryptographically encoded information. In example embodiments, the engineered cell includes one or more additional engineered modifications and wherein the cryptographically encoded information identifies the one or more additional engineered modifications. In example embodiments, the engineered cell is an adoptive cell therapeutic. In example embodiments, the engineered cell is a cell factory.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1D—Strategy for simultaneous multi-site genome editing. FIG. 1A shows an overview of the workflow for the design and evaluation of gRNAs. Base editor gRNAs are designed to target random genomic sites, and editing efficiency is evaluated through pooled cloning and transfection. Editing rates are analyzed and well-performing gRNAs are selected, enabling simultaneous multi-site base editing experiments. FIG. 1B shows a correlation of editing rate and transfected gRNA batch size. Editing rates were compared using the same gRNAs for different batch sizes of gRNA with the total concentration of gRNAs per transfection kept constant. Editing efficiencies were normalized to one, and the log-fold change of the editing rate was calculated. FIG. 1C shows a correlation between editing of gRNAs cloned and transfected in pools (y-axis) and subsequently selected gRNAs that were individually cloned, quantified, combined in a pool, and re-transfected (x-axis). FIG. 1D shows a replicate correlation of editing rates between sites for Adenine base editors (ABEs) and cytosine base editors (CBEs).

FIG. 2A-2D show multi-site base editing for information encoding. FIG. 2A shows, on the left: Binary information encoding through edited or unedited state, corresponding to is and 0s, respectively. The right side of FIG. 2A shows gRNAs are numbered and pools of either odd or even-numbered gRNAs are transfected. Edits are detected through targeted high throughput sequencing. FIGS. 2B, C show ROC (top) and precision-recall (bottom) curves for detection of gRNAs transfected in pools of 55 and 45 gRNAs each for CBE and ABE, respectively. FIG. 2D shows classification of editing outcomes with selected editing threshold. Editing above and below the threshold is colored in dark grey (1) and mid-grey (0), respectively, and false negatives (FNs) are colored in white and false positives (FPs) are colored in light grey.

FIG. 3A-3F show secure information transfer through asymmetric difficulty of detecting point mutations. FIG. 3A shows when the key is not known, whole genome sequencing (WGS) and variant calling software is required to detect installed edits. Possible outcomes are true positives (TPs), false negatives (FNs), false positives (FPs), and true negatives (TNs). FIG. 3B shows the rate of detection of an edited index site without a key depends on the false negative rate and false positive rate. The false negative rate depends on coverage and editing frequency, while the false positive rate depends on editing frequency. FIG. 3C shows breaking the key over multiple messages. The left side shows detection of TPs is obscured through false negative rates, but sites can be detected over multiple messages. The right side shows a distinction between false positives and true positives over multiple messages, with TPs shown in dark grey and FPs shown in light grey. FIG. 3D shows a minimum number of messages required to break the key for an adversary when not limited by sequencing coverage. FIG. 3E shows the sequencing cost to break the key over different editing frequencies and coverage levels (log 10). The coverage level required for breaking the code within 30 messages for each editing frequency is boxed in black. FIG. 3F shows the difference in cost in detecting the message without versus with a key over various allele frequencies.

FIG. 4A-4G shows a message encoding in cell signature and detection of perturbations through allelic frequencies. FIG. 4A shows an overview of application of GSE for application as encrypted cell signature for cell populations such as for cell lines or cell therapy. Cells carrying a mutation at the quality control (QC) site are spiked into a cell strain at a desired ratio, resulting in a defined editing frequency at the QC site. Under regular growth conditions, editing frequency at the QC site is maintained. In contrast, when cells are subjected to a bottleneck, the editing frequency is perturbed. FIG. 4B shows an absolute log 2 fold change (LFC) of editing frequency under regular passaging conditions. FIG. 4C shows an absolute log 2 fold change (LFC) of editing when bottlenecked to 50, 100, 500 or 1000 cells. FIG. 4D shows a fraction of sites with editing changes above different log 2 fold change (LFC) thresholds for cells after 4 passages, and cells that were to 50 and 500 cells, respectively. FIG. 4E shows the original message and errors occurring during encoding or decoding are shown above. The double errors represent the shift character for shifting to numeric values. FIG. 4F shows decoding of messages, showing the number of true positives (TP), false negatives (FN), and false positives (FP). FIG. 4G shows cell strains mixed with a strain that carries edit at QC site. ‘Defined edit (%)’ is the editing percentage as encoded in the message, ‘actual edit (%)’ is the experimentally observed percentage.

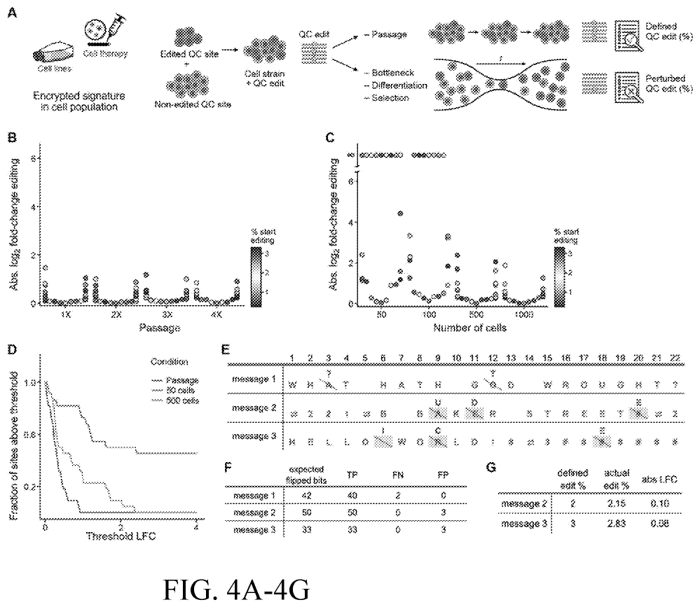

FIG. 5A-5D shows multi-site editing in individual embryonic stem cells. FIG. 5A shows an overview of the application of GSE for encrypted signatures in living animals. The encoding of a short message “Eureka” into mouse embryonic stem cells, which are routinely used for zygote injection and can thus be used for the generation of transgenic animals, was demonstrated. FIG. 5B shows an overview of workflow for obtaining individual editing cells. FIG. 5C shows editing across all sites before enrichment and after enrichment. FIG. 5D shows a histogram of the total number of edits in cells after enrichment for editing at a single site.

FIG. 6A-6B shows results of pooled gRNA cloning and screening. FIG. 6A shows out of 381 gRNAs that were cloned, efficient PCR amplification of 318 of the corresponding genomic sites was achieved. When transfected in batches of 48 gRNAs, 224 sites showed editing greater than 0.1%, and 137 sites showed editing greater than 0.5%. The background rates of the 138 sites with >0.5 editing (data not shown) and selected 111 sites with low background rates were further analyzed. FIG. 6B shows an editing distribution of 226 sites that showed >0.1% editing when transfected in batches of 48 gRNAs, before filtering out sites with high background.

FIG. 7 shows a correlation between editing rates for BE-hive prediction (assuming editing with a single gRNA for BE4 in HEK293 Ts) and observed editing rates in pools for selected sites. Pearson and Spearman correlation coefficient were calculated.

FIG. 8A-8B shows false negative rates at varying allele percentages and coverages obtained from calling variants on read files with artificially introduced mutations. FIG. 8A shows false negative rates obtained for Mutect2. FIG. 8B shows false negative rates obtained for VarScan2.

FIG. 9 shows a fraction of detected mutations from whole exome sequencing data at ~1000× coverage with variant caller Mutect2 and VarScan2, at ~1000× sequencing coverage.

FIG. 10 shows a false positive rates generated from whole exome sequencing. Whole exome sequencing was performed at 1000× coverage and variants were called using Mutect and VarScan at 1% and 0.5% allele frequency thresholds.

FIG. 11A-11B show a proportion of times a genomic index that is a true positive versus a false positive is called a variant. FIG. 11A shows a binomial distributions of the number of times key indices would be called variants over 200 messages at false negative rates (FNR) of 50, 75 and 85%. FIG. 11B shows binomial distributions of true positives and false positives and the proportion of times they are called variants over 10, 20 and 100 messages.

FIG. 12 shows a number of messages needed to reveal key indices using Varscan2 versus Mutect2. Values were calculated using a statistical analysis of how many messages are needed to meet an acceptable false positive rate of 100/(3*10) representing roughly the number of bits in a message over the size of the human genome and a false negative threshold of 0.1 meaning 90% of key indices have been discovered.

FIG. 13 shows attacker's cost when the optimal combination of sequence coverage and number of messages is chosen. Cost with unlimited messages is shown in black boxes; cost with messages limited to max. 30 messages is shown in grey boxes.

FIG. 14 shows the cost for an adversary to break a key when messages are encrypted at various allele frequencies. Cost for VarScan2 is shown by the top line and cost for Mutect2 is shown by the bottom line; at less than −2% allele frequency the cost approaches infinity when using Mutect2.

FIG. 15 shows a message authentication scheme. One of the key sites is specified as a message authentication site. A strain carrying a mutation at only this site is created, the editing frequency determined by sequencing, and the strain is then diluted into cell strains with encoded messages at a ratio achieving final editing frequencies as specified in the messages, e.g. in ‘HELLO WORD!#3’ the digit 3 specifies the editing frequency. Due to genetic drift, the editing frequency is expected to be perturbed when cells are subjected to a bottleneck compared to regular growth conditions, and the editing frequency can therefore inform about the integrity of the strain.

FIG. 16 shows a modified version of the five-bit International Telegraph Alphabet no. 2 (ITA2) for converting text to binary. ‘Shift character’ enables changing between state 1 and state 2.

FIG. 17 shows an increase in editing per site over two rounds of editing in a population of mESCs. Line depicts median editing values and points represent allelic editing frequencies per site.

FIG. 18 shows an editing increase at site with lowest editing efficiency in top 5% GFP expressing cells compared to all GFP positive cells.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a” “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present disclosure encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide methods editing, selecting, and enriching cells with multi-site edits. The methods help ensure that cells are isolated that contain a full set of desired edits. The present systems and methods can do this down to the single cell level. For many applications spanning agriculture, industrial biology, DNA cryptography, and cell-based therapeutics, assuring that individual cells have all the intended edits is critical. The embodiments disclosed herein establish parameters that may be used to enhance identification of and selection for fully edited cells. These methods significantly enhance the speed and efficiency with which complex engineered cells can be manufactured.

### Method of Enriching for Multi-Site Genome Editing

In one aspect, a method of enriching for multi-site edited cells comprises delivering to a population of cells a set of guide molecules and one or more nucleases, wherein the set of guide molecules directs the one or more programmable nuclease to introduce a set of edits to a plurality of genomic loci to generate an edited population of cells. The edited population of cells may then be screened for the presence of one or more enrichment marker. As demonstrated herein, the enrichment markers allow for prediction and/or verification that a given cell has all edits without requiring verification at each edited locus. Cells identified as having the one or more enrichment markers may then be isolated ensuring a sub-population of cells comprising the full set of intended edits. The methods disclosed herein may be carried out in a single delivery reaction and allow for rapid screening and enrichment of isolated cells.

### Generating a Population Of Edited Cells

A set of guide molecules, as referred to herein as a “guide library”, may be used in the context of the present disclosure to target multiple genomic loci. In example embodiments, the pool of randomized guide molecules for multi-site genome editing comprises a set of guide molecules. In general, multiple guide molecules, corresponding to multiple unique genomic loci, are introduced to the plurality of genomic loci. For systems using programmable nucleases guided by guide molecules, the programmable nuclease can then be directed to multiple genomic loci simultaneously. For example, multiple edits may be carried out in parallel.

In example embodiments, the set of guide molecules is at least two unique guide molecules. A unique guide molecule comprises a guide molecule designed to direct a programmable nuclease to a target genomic locus. Two or more guide molecules are unique if they direct a programmable nuclease to different genomic loci. A set of at least two unique guide molecules refers to multiple guide molecules (e.g., 2, 3, 4, 5, 10, 100, 1000, 10,000, etc.,) corresponding to the at least two unique guide molecules. For example, a set of four guide molecules with two unique guide molecules X and Y could correspond to a set of 2X and 2Y or 1X and 3Y or 3X and 1Y. Therefore, in larger examples, any combination of unique guide molecules in a set is considered. In example embodiments, the set of guide molecules comprise of at least 5, 10, 20, 30, 40, 50, 100, 200, 300, 400, 500, or 1000 unique guide molecules. In example embodiments, the set of guide molecules comprise between 10 and 1000, 10 and 500, 10 and 250, 10 and 100, 10 and 50, 50 and 1000, 50 and 500, 50 and 250, 50 and 100, 100 and 1000, 100 and 500, 100 and 250, 100 and 200, 250 and 1000, 250 and 500, or 500 and 1000 unique guide molecules. In example embodiments, the set of guide molecules may comprise of 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140 unique guide molecules.

### Guide Molecules

A guide molecule comprises a guide molecule and a scaffold. The guide molecule represents a reprogrammable aspect of the guide molecule and may be set to hybridize with a desired target sequence at a given genomic loci. The genomic loci may constitute a coding or non-coding sequence. The scaffold portion of the guide molecule interfaces with the programmable nuclease to form a complex comprising the guide molecule and the programmable nuclease.

In some embodiments the system includes two guide molecules that can each be splint or bridge molecules. In some embodiments, the first and second guide molecules comprise a region capable of hybridizing to a cleaved strand of the target polynucleotide and a region capable of hybridizing to the donor sequence. In some embodiments, the composition comprises a splint oligonucleotide that has a region capable of hybridizing to a cleaved strand of the target polynucleotide and a region capable of hybridizing to the donor molecule.

The ability of a guide molecule to directly bind the one or more programmable nucleases to a target nucleic acid sequence may be assessed by any suitable assay. For example, the components of one or more programmable nucleases may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of the one or more programmable nucleases, including the guide molecule to be tested and a control guide molecule different from the test guide molecule, and comparing binding or rate of cleavage at the target sequence between the test and control guide molecule reactions. Other assays are possible and will occur to those skilled in the art.

In some embodiments, the guide molecule is an RNA. The guide molecule(s) are included in the one or more programmable nucleases and has sufficient complementarity with a target nucleic acid sequence to hybridize with the target nucleic acid sequence and direct sequence-specific binding of a nucleic acid-targeting complex to the target nucleic acid sequence. The degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), Clustal W, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net).

A guide molecule may be selected to target any target nucleic acid sequence in a target polynucleotide. In one embodiment, the target polynucleotide may be DNA. In one embodiment, the target polynucleotide is an RNA polynucleotide. The target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In one embodiment, the target sequence may be a sequence within an RNA molecule selected from the group consisting of mRNA, pre-mRNA, and rRNA. In one embodiment, the target sequence may be a sequence within an RNA molecule selected from the group consisting of ncRNA, and lncRNA. In one example embodiment, the target sequence may be a sequence within an mRNA molecule or a pre-mRNA molecule.

In one embodiment, the scaffold is located 5′ of the guide molecule. In one embodiment, the scaffold is located 3′ of the guide molecule. The direct repeat may comprise one or more modifications. The modifications may remove unnecessary secondary structure or otherwise minimize the overall size of the scaffold component of the guide molecule. The direct repeat may have one or more modifications that increase the stability of the guide molecule, enhance complex formation with the Cas polypeptides described herein, for example by modulating nuclease activity (either by increasing or decreasing), and/or reduce off-target effects.

In certain embodiments, the guide molecule length of the guide molecule is from 15 to 35 nt. In certain embodiments, the guide molecule length of the guide molecule is at least 15 nucleotides. In certain embodiments, the guide molecule length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

In some embodiments, the degree of complementarity between a guide molecule and its corresponding target sequence can be about or more than about 50%, 60%, 75%, 80%, 85%, 90%, 95%, 97.5%, 99%, or 100%; a guide molecule can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or a guide molecule can be less than about 75, 50, 45, 40, 35, 30, 25, 20, 15, 12, or fewer nucleotides in length. In some embodiments, the degree of complementarity between a guide molecule and its corresponding target sequence is greater than 94.5% or 95% or 95.5% or 96% or 96.5% or 97% or 97.5% or 98% or 98.5% or 99% or 99.5% or 99.9% or is 100%. In some embodiments, off-target complementarity is less than 100% or 99.9% or 99.5% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% between the off-target sequence and the guide molecule. In example embodiments the off-target complementarity is 0%, or 0.5%, or 1%, or 1.5%, or 2%, or 2.5%, or 3%, or 3.5%, or 4%, or 4.5%, or 5%, or 5.5%, or 6%, or 6.5%, or 7%, or 7.5%, or 8%, or 8.5%, or 9%, or 9.5%, or 10%, or 10.5%, or 11%, or 11.5%, or 12%, or 12.5%, or 13%, or 13.5%, or 14%, or 14.5%, or 15%, or 15.5%, or 16%, or 16.5%, or 17%, or 17.5%, or 18%, or 18.5%, or 19%, or 19.5%, or 20%, or 20.5%, or 21%, or 21.5%, or 22%, or 22.5%, or 23%, or 23.5%, or 24%, or 24.5%, or 25%, or 25.5%, or 26%, or 26.5%, or 27%, or 27.5%, or 28%, or 28.5%, or 29%, or 29.5%, or 30%, or 30.5%, or 31%, or 31.5%, or 32%, or 32.5%, or 33%, or 33.5%, or 34%, or 34.5%, or 35%, or 35.5%, or 36%, or 36.5%, or 37%, or 37.5%, or 38%, or 38.5%, or 39%, or 39.5%, or 40%, or 40.5%, or 41%, or 41.5%, or 42%, or 42.5%, or 43%, or 43.5%, or 44%, or 44.5%, or 45%, or 45.5%, or 46%, or 46.5%, or 47%, or 47.5%, or 48%, or 48.5%, or 49%, or 49.5%, or 50%, or 50.5%, or 51%, or 51.5%, or 52%, or 52.5%, or 53%, or 53.5%, or 54%, or 54.5%, or 55%, or 55.5%, or 56%, or 56.5%, or 57%, or 57.5%, or 58%, or 58.5%, or 59%, or 59.5%, or 60%, or 60.5%, or 61%, or 61.5%, or 62%, or 62.5%, or 63%, or 63.5%, or 64%, or 64.5%, or 65%, or 65.5%, or 66%, or 66.5%, or 67%, or 67.5%, or 68%, or 68.5%, or 69%, or 69.5%, or 70%, or 70.5%, or 71%, or 71.5%, or 72%, or 72.5%, or 73%, or 73.5%, or 74%, or 74.5%, or 75%, or 75.5%, or 76%, or 76.5%, or 77%, or 77.5%, or 78%, or 78.5%, or 79%, or 79.5%, or 80%, or 80.5%, or 81%, or 81.5%, or 82%, or 82.5%, or 83%, or 83.5%, or 84%, or 84.5%, or 85%, or 85.5%, or 86%, or 86.5%, or 87%, or 87.5%, or 88%, or 88.5%, or 89%, or 89.5%, or 90%, or 90.5%, or 91%, or 91.5%, or 92%, or 92.5%, or 93%, or 93.5%, or 94%, or 94.5%, or 95%, or 95.5%, or 96%, or 96.5%, or 97%, or 97.5%, or 98%, or 98.5%, or 99%, or 99.5%, with it being advantageous that the off-target complementarity is less than 0.5%, or 1%, or 1.5%, or 2%, or 2.5%, or 3%, or 3.5%, or 4%, or 4.5%, or 5%, or 5.5%, or 6%, or 6.5%, or 7%, or 7.5%, or 8%, or 8.5%, or 9%, or 9.5%, or 10%, or 10.5%, or 11%, or 11.5%, or 12%, or 12.5%, or 13%, or 13.5%, or 14%, or 14.5%, or 15%, or 15.5%, or 16%, or 16.5%, or 17%, or 17.5%, or 18%, or 18.5%, or 19%, or 19.5%, or 20%, or 2.5%, or 21%, or 21.5%, or 22%, or 22.5%, or 23%, or 23.5%, or 24%, or 24.5%, or 25%, or 25.5%, or 26%, or 26.5%, or 27%, or 27.5%, or 28%, or 28.5%, or 29%, or 29.5%, or 30%, or 3.5%, or 31%, or 31.5%, or 32%, or 32.5%, or 33%, or 33.5%, or 34%, or 34.5%, or 35%, or 35.5%, or 36%, or 36.5%, or 37%, or 37.5%, or 38%, or 38.5%, or 39%, or 39.5%, or 40%, or 4.5%, or 41%, or 41.5%, or 42%, or 42.5%, or 43%, or 43.5%, or 44%, or 44.5%, or 45%, or 45.5%, or 46%, or 46.5%, or 47%, or 47.5%, or 48%, or 48.5%, or 49%, or 49.5%, or 50%, or 50.5%, or 50.5%, or 51%, or 51.5%, or 52%, or 52.5%, or 53%, or 53.5%, or 54%, or 54.5%, or 55%, or 55.5%, or 56%, or 56.5%, or 57%, or 57.5%, or 58%, or 58.5%, or 59%, or 59.5%, or 60%, or 6.5%, or 61%, or 61.5%, or 62%, or 62.5%, or 63%, or 63.5%, or 64%, or 64.5%, or 65%, or 65.5%, or 66%, or 66.5%, or 67%, or 67.5%, or 68%, or 68.5%, or 69%, or 69.5%, or 70%, or 7.5%, or 71%, or 71.5%, or 72%, or 72.5%, or 73%, or 73.5%, or 74%, or 74.5%, or 75%, or 75.5%, or 76%, or 76.5%, or 77%, or 77.5%, or 78%, or 78.5%, or 79%, or 79.5%, or 80% between the target sequence and the guide molecule.

Many modifications to guide molecules are known in the art and are further contemplated within the context of this disclosure. Various modifications may be used to increase the specificity of binding to the target sequence and/or increase the activity of the Cas protein and/or reduce off-target effects. Example guide molecule modifications are described in International Patent Application No. PCT/US2019/045582, specifically paragraphs [0178]-[0333], which is incorporated herein by reference as if expressed in its entirety herein. Additional guide molecule modifications are described in detail below.

The guide molecule may be designed to reduce the degree secondary structure within the guide molecule. In some embodiments, about or less than about 75%, 50%, 40%, 30%, 25%, 20%, 15%, 10%, 5%, 1%, or fewer of the nucleotides of the guide molecule participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151-62).

The guide molecule is configured to minimize or reduce off-target effects. Guide molecules and strategies to minimize toxicity and off-target effects can be as in WO 2014/093622 (PCT/US2013/074667); or, via mutation as described herein.

In many instances, the guide molecule is randomized. For certain screening methods or encryption methods, as described herein, randomized guide molecules can be used with one or more programmable nuclease to edit multiple genomic loci. For example, using a randomized set of guide molecules can be used for the unbiased design of efficient targeted guide molecules, operability of genome-editing products, and encoding of data. Generating/preparing randomized guide molecules may comprise any method of randomization and guide molecule design. For example, a set of random genomic loci can be generated using any random number generation technique. Then guide molecules are designed around the randomly generated target loci using any known method in the art and described herein. Finally, the guide molecules are prepared according to the design and any known method in the art and described herein resulting in randomized guide molecules.

### Programmable Nuclease

As used herein, a programmable nuclease refers to a nuclease capable of forming a complex with said guide molecule and wherein the site of nuclease activity on a genomic loci is dictated by the guide molecule. A programmable nuclease may comprise a CRISPR-Cas system or a component thereof (e.g., a Cas nuclease) or an OMEGA system or a component thereof (e.g., an IscB nuclease, an IsrB nucelase, an IshB nuclease, a TnpB nuclease, a Fanzor, etc.) (see e.g., Altae-Tran, H.; et al. The Widespread IS200/IS605 Transposon Family Encodes Diverse Programmable RNA→Guided Endonucleases. Science, 2021, 374:57-65; Karvelis et al., Nature, 599: 692-696 (2021); Hirano et al., Nature, 610:575-581 (2022); and Saito et al., Nature, 620:660-668 (2023); Jiang et al., Science Advances, 9(39), DOI: 10.1126/sciadv.adk0171 (2023)). The same programmable nuclease may be used with the entire set of guide molecules, or the set of molecules may comprise one or more guides capable of complexing with different types of programmable nucleases.

### CRISPR-Cas Systems

The programmable nuclease may be a Cas nuclease. CRISPR-Cas systems can generally fall into two classes based on their architectures of their effector molecules, which are each further subdivided by type and subtype. The two classes are Class 1 and Class 2. Class 1 CRISPR-Cas systems have effector modules composed of multiple Cas proteins, some of which form crRNA-binding complexes, while Class 2 CRISPR-Cas systems include a single, multi-domain crRNA-binding protein.

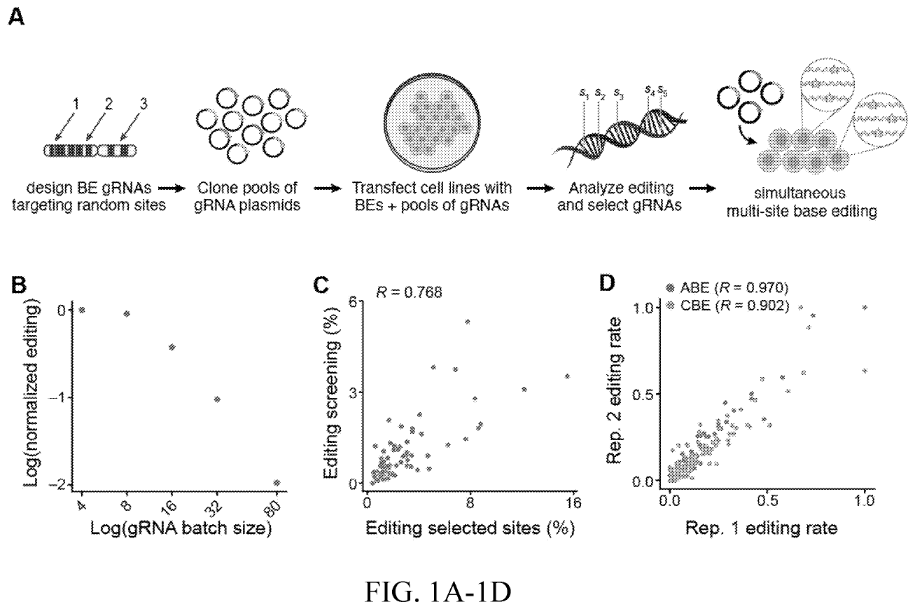

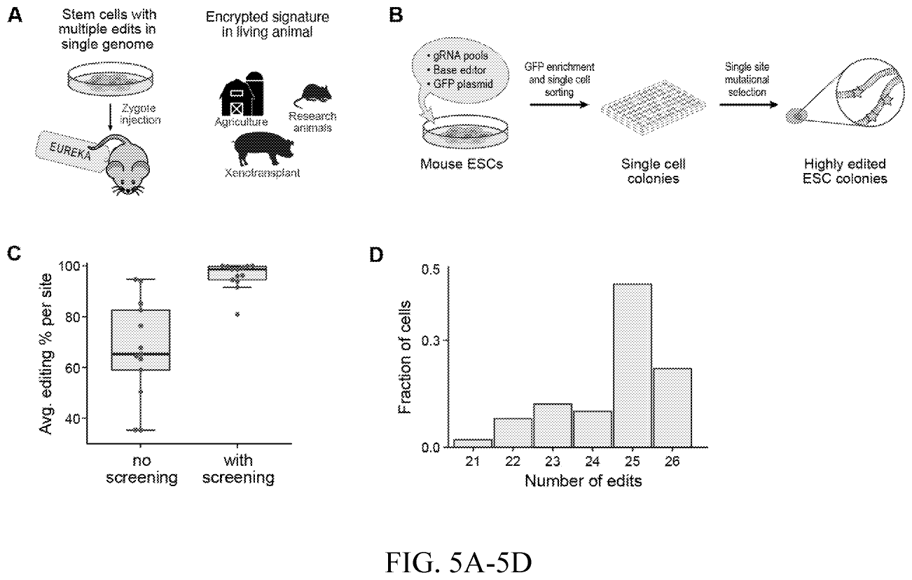

In some embodiments, the CRISPR-Cas system that can be used to modify a polynucleotide as described herein can be a Class 1 CRISPR-Cas system. Class 1 CRISPR-Cas systems are divided into types I, III, and IV. Makarova et al. 2020. Nat. Rev. 18: 67-83, particularly as described in FIG. 1. Type I CRISPR-Cas systems are divided into 9 subtypes (I-A, I-B, I-C, I-D, I-E, I-F1, I-F2, I-F3, and I-G). Makarova et al., 2020. Class 1, Type I CRISPR-Cas systems can contain a Cas3 protein that can have helicase activity. Type III CRISPR-Cas systems are divided into 6 subtypes (III-A, III-B, III-C, III-D, III-E, and III-F). Type III CRISPR-Cas systems can contain a Cas10 that can include an RNA recognition motif called Palm and a cyclase domain that can cleave polynucleotides. Makarova et al., 2020. Type IV CRISPR-Cas systems are divided into 3 subtypes. (IV-A, IV-B, and IV-C). Makarova et al., 2020. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al. 2018. The CRISPR Journal, v. 1, n5, FIG. 5.

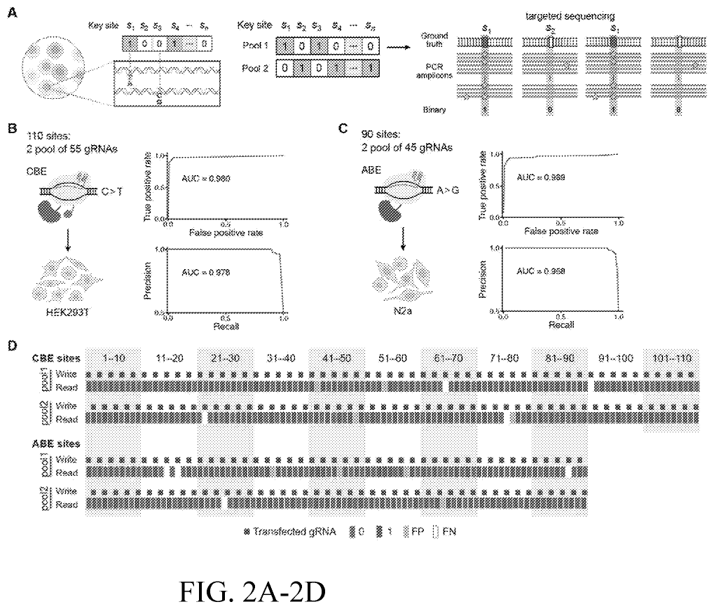

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type VI systems can be divided into 5 subtypes: VI-A, VI-B1, VI-B2, VI-C, and VI-D.

### OMEGA Nucleases

The programmable nuclease may be an OMEGA nuclease. The OMEGA nuclease may be an IscB nuclease, an IsrB nuclease, a TnpB nuclease, or a Fanzor nuclease.

### IscB Nuclease

In one embodiment, IscB nucleases may comprise a split RuvC nuclease domain comprising RuvC-1, RuvC-II, and RuvC-III subdomains. Some IscB proteins may further comprise a HNH endonuclease domain. In one example embodiment, the RuvC endonuclease domain is split by the insertion of a bridge helix, a HNH domain, or both. However, unlike Cas9, IscB nucleases do not contain a Rec domain. In addition, IscB nucleases may further comprise a conserved N-terminal domain (also referred to herein as a PLMP domain), which is not present in Cas9 proteins. IscB proteins may also further comprise a conserved C-terminal domain. In one example embodiment, an IscB nuclease comprises, moving from the N- to C-terminus, a PLMP domain, a RuvC-I subdomain, a bridge helix, a RuvC-II subdomain, a HNH domain, a RuvC-III subdomain, and a C terminal domain. In one embodiment, IscB nucleic acid-guided nucleases may comprise CRISPR-associated IscB nucleases. In one embodiment, the IscB nucleases are CRISPR-associated proteins, e.g., the loci of the nucleases are associated with an CRISPR array. In one embodiment the IscBs may be referred to as Cas IscBs. The Cas IscB nucleic acid-guided nuclease may comprise one or more domains, e.g., one or more of a X domain (e.g., at N-terminus), a RuvC domain, a Bridge Helix domain, and a Y domain (e.g., at C-terminus). See International Application Publication No. WO 2022/087494 A1 incorporated herein by reference in its entirety.

### IsrB Nuclease

IsrBs are homologs of IscB nucleases. IsrB nucleases comprise the PLMP and RuvC domains but do not comprise a HNH domain. In one embodiment, the IsrB nuclease comprises a PLMP domain and a split RuvC but lacks the HNH domain present between the RuvC-II and III subdomains in IscB nucleases. In one embodiment, the IsrB is an OMEAG RNA guided nickase. In one embodiment, the OMEAG RNA guided IsrB nicks a DNA target. In one embodiment, the DNA target is a dsDNA and the nicks occurs on the non-target strand of the dsDNA target. In one embodiment, the IsrB nicks the dsDNA in a guide and TAM specific manner. Accordingly, applications where a nickase is utilized can be used with the IsrB nucleases detailed herein in a manner functionally similar to an IscB that has been inactivated at the HNH domain.

### IshB Nuclease

As noted above IshBs are IscB homologs and may be referred to herein as an Insertion sequence HNH-like OrfB (IshB) nuclease. IshB nucleases are generally smaller than IsrB or IscB nucleases and contain only the PLMP and HNH domain, but no RuvC domain. In one embodiment, the IshB, or IscB homolog, comprises a PLMP domain and an HNH domain, but does not comprise a RuvC domain.

Some IshB nucleases may be part of the IS605 OrfB family of transposases. In an embodiment, the IshB nuclease is from Actinoplanes lobatus and has the Genbank accession number MBB4752409. In an embodiment, the RefSeq database accession number for the nuclease with accession number MBB4752409 is WP_188124268 and the INSDC number is GGN95087.

### TnpB Nuclease

In one aspect, embodiments disclosed herein are directed to compositions comprising a TnpB and an OMEGA RNA capable of forming a complex with the TnpB and directing site-specific binding of the TnpB to a target sequence on a target polynucleotide.

TnpB nucleases may comprise a Ruv-C-like domain. Exemplary TnpB sequences are shown in FIG. 1, Table 1A, Table 1B, Table 1C and Table 5 of International Patent Publication Application No. WO 2022/159892 A1, herein incorporated by reference in its entirety. The RuvC domain may be a split RuvC domain comprising RuvC-I, RuvC-II, and RuvC-III subdomains. The TnpB may further comprise one or more of a HTH domain, a bridge helix domain and a zinc finger domain. TnpB nucleases do not comprise an HNH domain. In one example embodiment, TnpB proteins comprise, starting at the N-terminus a HTH domain, a RuvC-I sub-domain, a bridge helix domain, a RuvC-II sub-domain, a zinger finger domain, and a RuvC-III sub-domain. In one example embodiment, the RuvC-III sub-domain forms the C-terminus of the TnpB nuclease.

In one embodiment, the TnpB nuclease is from Epsilonproteobacteria bacterium, or Actinoplanes lobatus strain DSM 43150, Actinomadura celluolosilytica strain DSM 45823, Actinomadura namibiensis strain DSM 44197, Alicyclobacillus macrosprangiidus strain DSM 17980, Lipingzhangella halophila strain DSM 102030, or Ktedonobacter recemifer. In one embodiment, the TnpB nuclease is from Ktedonobacter racemifer, or comprises a conserved RNA region with similarity to the 5′ ITR of K. racemifer TnpB loci.

### Fanzor Nuclease

In one aspect, embodiments disclosed herein are directed to compositions comprising an engineered Fanzor and/or OMEGA RNA capable of forming a complex with the Fanzor and directing site-specific binding of the Fanzor to a target sequence on a target polypeptide.

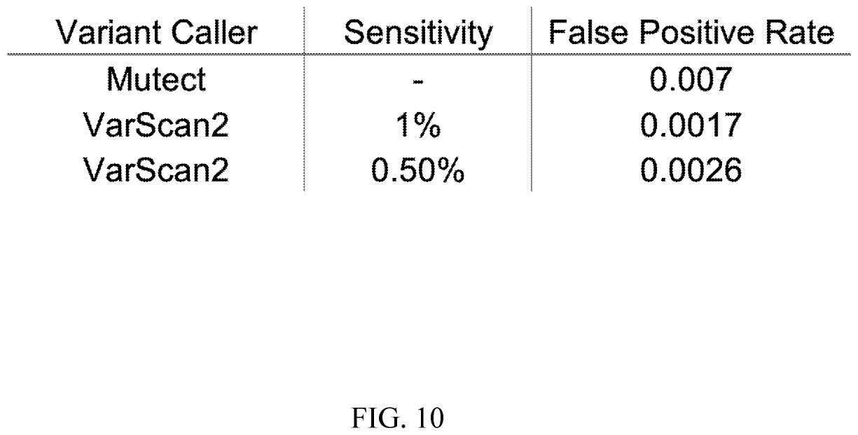

Fanzor nucleases may comprise a Ruv-C-like domain. Exemplary Fanzor sequences are shown or encoded by those in Table 1, Table 6, Table 7, Table 8, Table 9, Table 10, Table 11, Table 12, Table 13, Table 14, and FIG. 20 of International Patent Application Publication No. WO 2023/114872 A3, herein incorporated by reference in its entirety. In some embodiments, the Fanzor nuclease is a nuclease as shown and described in relation with FIGS. 10C-10E, FIG. 35, and FIG. 56A-56D of International Patent Application Publication No. WO 2023/114872 A3. The RuvC domain may be a split RuvC domain comprising a RuvC-I, RuvC-II, and RuvC-III subdomains. The Fanzor may further comprise one or more of a HTH domain, a bridge helix domain, a REC domain, a zinc finger domain, or any combination thereof. Fanzor nucleases do not comprise an HNH domain. In one example embodiment, Fanzor proteins comprise, starting at the N-terminus a HTH domain, a RuvC-I sub-domain, a bridge helix domain, a RuvC-II sub-domain, a zinger finger domain, and a RuvC-III sub-domain. In one example embodiment, the RuvC-III sub-domain forms the C-terminus of the Fanzor nuclease.

Example programmable nuclease systems are discussed in further detail below.

### DNA and RNA Base Editing

The present disclosure also provides for base editing systems. As used herein, “base editing” refers generally to the process of polynucleotide modification via a nucleic acid-guided nuclease (such as a programmable nuclease, including but not limited to, a CRISPR-Cas-based or Cas-based) system that does not include excising nucleotides to make the modification. Base editing can convert base pairs at precise locations without generating excess undesired editing byproducts that can be made using traditional CRISPR-Cas systems. Accordingly, in one example embodiment, the base editing system edits the target gene to reduce or eliminate its expression or to increase its expression. The methods and systems described herein allow for multi-site base editing through pooled editing. Combining base editors with pooled editing allows for one-step multi-site polynucleotide modification. Introducing multiple base edits in a single genome offers the ability to interrogate polynucleotide interactions at single-base resolution or at a gene level through stop codon generation. In example embodiments, base editing systems introduce multiple simultaneous polynucleotide edits.

In some embodiments, the base editing system is a DNA base editing system. In some embodiments, the base editing system is an RNA base editing system. In some embodiments, the base editing system is a DNA and RNA base editing system. In general, such a system may comprise a nucleotide deaminase (e.g., an adenosine deaminase or cytidine deaminase) fused or otherwise coupled with a programmable nuclease, e.g., Cas protein or other nuclease protein. The Cas protein may be a dead Cas protein or a Cas nickase protein. In certain examples, the system comprises a mutated form of an adenosine deaminase fused with a dead CRISPR-Cas or CRISPR-Cas nickase. The mutated form of the adenosine deaminase may have both adenosine deaminase and cytidine deaminase activities. The present disclosure also provides for compositions that include nucleotide sequence(s) comprising encoding sequences for one or more components of a base editing system.

The base editing systems may be capable of modifying a single nucleotide in a target polynucleotide. The modification may be a G→A or C→T point mutation, a T→C or A→G point mutation. The modification may repair or correct a pathogenic SNP. Accordingly, the compositions and systems may remedy a disease caused by a G→A or C→T point mutation, a T→C or A→G point mutation, or a pathogenic SNP. The modification may introduce a pathogenic SNP, such as in the context of generating a disease model. Accordingly, the compositions and systems of the present disclosure may be used in the context of disease modeling and/or drug screening.

In one example embodiment, a method of multi-site polynucleotide (e.g., gene, genome, transcript, or transcriptome) editing comprises administering a DNA or RNA base editing system to modify one or more targets of the randomized guide molecules. In some embodiments, the modification either decreases expression of one or more targets of the randomized guide molecules or increases expression of the one or more targets of the randomized guide molecules.

In one example embodiment, the nucleotide deaminase may be a DNA base editor used in combination with a programmable nuclease or system. In some embodiments, the programmable nuclease is a DNA binding programmable nuclease. In some embodiments, the DNA binding programmable nuclease is a Cas protein such as, but not limited to, Class 2 Type II and Type V systems. Two classes of DNA base editors are generally known: cytosine base editors (CBEs) and adenine base editors (ABEs). CBEs convert a C·G base pair into a T·A base pair (Komor et al. 2016. Nature. 533:420-424; Nishida et al. 2016. Science. 353; and Li et al. Nat. Biotech. 36:324-327) and ABEs convert an A·T base pair to a G·C base pair. Collectively, CBEs and ABEs can mediate all four possible transition mutations (C to T, A to G, T to C, and G to A). Rees and Liu. 2018. Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some embodiments, the programmable nuclease can be a catalytically disabled variant, such as a nickase. In some systems, the catalytically disabled programmable nuclease is a Cas (or other programmable nuclease described elsewhere herein) variant or modified Cas (or other programmable nuclease described elsewhere herein) that can have nickase functionality and can generate a nick in the non-edited DNA strand to induce repair of the non-edited strand using the edited strand as a template. Komor et al. 2016. Nature. 533:420-424; Nishida et al. 2016. Science. 353; and Gaudeli et al. 2017. Nature. 551:464-471.

Other Example Type V base editing systems that can be used with the present disclosure are described in International Patent Publication Nos. WO 2018/213708, WO 2018/213726, and International Patent Applications Nos. PCT/US2018/067207, PCT/US2018/067225, and PCT/US2018/067307, each of which is incorporated herein by reference in its entirety.

In one example embodiment, the base editing system may be an RNA base editing system. As with DNA base editors, a nucleotide deaminase capable of converting nucleotide bases may be fused to a Cas protein or other programmable nuclease described elsewhere herein. In these embodiments, the Cas (or other programmable nuclease) protein is capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA base editor may be used to delete or introduce a post-translation modification site in the expressed mRNA. In contrast to DNA base editors, whose edits are permanent in the modified cell, RNA base editors can provide edits where finer, temporal control may be needed, for example in modulating a particular immune response. Example Type VI RNA-base editing systems are described in Cox et al. 2017. Science 358: 1019-1027, International Patent Publication Nos. WO 2019/005884, WO 2019/005886, and WO 2019/071048, and International Patent Application Nos. PCT/US20018/05179 and PCT/US2018/067207, which are incorporated herein by reference in their entirety. An example FnCas9 system that may be adapted for RNA base editing purposes is described in International Patent Publication No. WO 2016/106236, which is incorporated herein by reference in their entirety.

An example method for delivery of base-editing systems, including use of a split-intein approach to divide CBE and ABE into reconstitutable halves, is described in Levy et al. Nature Biomedical Engineering doi.org/10.1038/s41441-019-0505-5 (2019), which is incorporated herein by reference.

### Example DNA Base Editing Modifications to Increase or Decrease Expression of Target Polynucleotides

In one embodiment, a DNA base editing system may be configured to make one or more base edits in one or more non-coding regions of a gene or genome. In one embodiment, a DNA base editing system may be configured to make one or more base edits in one or more coding regions of a gene or genome. In one embodiment, a DNA base editing system may be configured to make one or more base edits in one or more coding regions of a gene or genome and one or more non-coding regions of a gene or genome. In some embodiments, a DNA base editing system may be configured to make one or more base edits in one or more targets of the one or more randomized guide molecules. In some embodiments, the one or more targets of the one or more randomized guide molecules are in one or more coding regions of a gene or genome. In some embodiments, the one or more targets of the one or more randomized guide molecules are in one or more non-coding regions of a gene or genome. In some embodiments, the one or more targets of the one or more randomized guide molecules are in one or more non-coding regions of a gene or genome and in one or more coding regions of a gene or genome.

DNA Base-Editing Modifications that Decrease Expression by Targeting Non-Coding Regions

In one embodiment, a DNA base editing system may be configured to make one or more base edits in a coding and/or non-coding region of a gene or genome, where the base edits are made within one or more targets of the randomized guide molecules of the DNA base editing system. In some embodiments, the one or more targets of the randomized guide molecules are in one or more enhancer regions of one or more genes. Thus, in one embodiment, the one or more base edits introduce mutations in an enhancer region controlling expression of one or more genes containing one or more targets of the randomized guide molecules so as prevent or disrupt the binding of transcription factors such that transcription initiation and gene expression are blocked or reduced. In some embodiments, the one or more targets of the randomized guide molecules are in one or more promoter regions controlling expression of one or more genes. Thus, in one example embodiment, the one or more base edits introduce mutations in a promoter region controlling expression of one or more genes containing one or more targets of the randomized guide molecules to prevent or disrupt the binding of transcription factors and/or RNA polymerase such that transcription initiation and gene expression are blocked or reduced. In some embodiments, the one or more targets of the randomized guide molecules are in a silencing region controlling expression of one or more genes containing one or more targets of the randomized guide molecules. Thus, in one example embodiment, the base editor is configured to introduce one or more base edits that introduce a new silencing region or modify and strengthen an existing silencing region controlling expression of one or more genes that contain one or more targets for the one or more randomized guide molecules thereby leading to the recruitment of transcriptional repressors that block or decrease gene expression. In another embodiment, the base editor is configured to make one or more base edits that disrupt one or more insulator sequences controlling expression of one or more genes containing one or more targets of the randomized guide molecules such that nearby silencer elements or repressive chromatin structures are able to decrease gene expression. In one embodiment, a DNA base editing system may be configured to make one or more base edits in a non-coding region of one or more genes containing one or more targets of the randomized guide molecules that result in increased expression of the one or more genes.

DNA Base-Editing Modifications that Increase Expression by Targeting Non-Coding Regions

In one embodiment, a DNA base editing system may be configured to make one or more base edits in a non-coding region of one or more genes containing one or more targets of the randomized guide molecules that result in increased expression of the one or more genes containing one or more targets of the randomized guide molecules. In one embodiment, the base editing system is configured to introduce one or more base edits in one or more enhancer regions controlling expression of one or more genes containing one or more targets of the randomized guide molecules such that binding of transcription factors or other regulatory proteins is increased or strengthened and gene expression increased. In another embodiment, the base editing system is configured to introduce one or more base edits in one or more promoter regions controlling expression of one or more genes containing one or more targets of the randomized guide molecules such that binding of transcription factors and/or RNA polymerase is increased or strengthened and gene expression is increased. In another embodiment, the base editing system is configured to introduce one or more base edits that disrupt or remove one or more silencer elements that control expression of one or more genes containing one or more targets of the randomized guide molecules, such that binding of transcriptional repressors is prevented or weakened and gene expression is increased. In another embodiment, the base editing system is configured to introduce or strengthen insulator sequences controlling expression of the one or more genes containing one or more targets of the randomized guide molecules, thereby reducing the influence of nearby silencer elements or repressive chromatin structures such that gene expression is increased.

DNA Base-Editing Modifications that Decrease Expression by Targeting Coding Regions

In one embodiment, a DNA base editing system may be configured to make one or more base edits in a coding region of one or more genes containing one or more targets of the randomized guide molecules that result in decreased expression of the one or more genes containing one or more targets of the randomized guide molecules. In one embodiment, the one or more base edits result in a frame-shift mutation leading to introduction of a premature stop codon and the production of non-functional truncated gene products, or the triggering of nonsense-mediated mRNA decay (NMD), thereby resulting in reduced expression or gene product activity. In another embodiment, the one or more base edits result in introduction of a premature stop codon within the coding region resulting in production of truncated non-functional proteins or the triggering of NMD, thereby resulting in reduced gene expression or gene product activity. In another embodiment, the one or more base edits target specific functional domains within the coding region to create mutations that impair the function of the gene product. While this approach may not directly decrease gene expression, it can lead to the production of non-functional proteins, effectively resulting in a loss-of-function effect. In another embodiment, the one or more base edits may introduce mutations in the coding region at exon-intron boundaries or splice sites leading to aberrant splicing, production of non-function proteins or triggering NMD and thereby reducing gene expression or activity of a resulting gene product. In another embodiment, the one or more base edits may target regulatory elements within the coding regions that affect gene expression, such as internal ribosome entry sites (IRES). One or more modifications may be made at these regulatory elements to reduce gene expression. In another embodiment, the one or more base edits may introduce, change, or remove a sequence encoding a post-translation modification (PTM) site in the expressed gene product. Post-translational modification, such as phosphorylation, glycosylation, or ubiquitination, play an essential role in regulating protein function, stability and localization. Post-translation modification may be both necessary to inhibit a protein's functions or to activate a protein's function. Accordingly, modifications that introduce inhibitory PTMs or remove activating PTMs may be made to decrease protein function, stability, and/or degradation.

DNA Base-Editing Modifications that Increase Expression by Targeting Coding Regions

In one embodiment, the base editing system is configured such that one or more base edits are made in a coding region of the one or more genes containing one or more targets of the randomized guide molecules such that expression of the one or more genes is increased. In one embodiment, the one or more base edits comprise removing or disrupting inhibitor sequences, such as IRESs or upstream open reading frames (uORFs), which can negatively affect expression. In one embodiment, the one or more base edits may comprise introducing specific mutations within the coding region that can potentially improve protein stability, folding, or resistance to degradation. While this does not directly increase gene expression, it can lead to higher protein levels and enhanced function. In one embodiment, the one or more base edits may comprise removal or disruption of a sequence encoding an inhibitory PTM site, removal or disruption of one or more ubiquitination sites, or introduction of PTM sites that stabilize or enhance protein function. In one embodiment, the one or more base edits may comprise mutations or modifications within the coding region that improve catalytic activity, binding affinity, or other functional properties of the protein. This approach does not directly increase gene expression but can result in an overall increase in the functional output of the gene product.

### Example RNA Base Editing Modifications to Increase or Decrease Expression of Target Genes

RNA base editors allow for targeted RNA editing without modifying the underlying DNA sequence. Such systems may be useful where more temporal and/or spatial control of gene expression is desired.

In one embodiment, an RNA base editing system is used to introduce one or more base edits to one or more RNA molecules transcribed from one or more genes containing one or more targets of the randomized guide molecules, such that expression or activity of the gene product is reduced. In one embodiment, the one or more base edits introduce a frame-shift mutation leading to introduction of a premature stop code, resulting in production of a truncated protein or triggering NMD, both which lead to decreased gene expression. In another embodiment, the one or more base edits introduce splice sites or splice regulatory elements that lead to aberrant splicing and production of non-functional proteins or mRNA that is degraded through NMD, thereby decreasing gene expression. In one embodiment, the one or more base edits target specific functional domains of the gene product encoded within the mRNA that impair the function of the gene product. While this approach may not directly decrease translation of the mRNA, it leads to the production of a non-functional gene product or gene products with decreased function, effectively achieving a loss-of-function effect. In one embodiment, the one or more base edits modify regulatory elements within the mRNA. Some mRNAs have regulatory elements that can affect gene expression, such as upstream open reading frames (uORFs) or IRESs, and disrupting these elements may reduce gene expression. In one embodiment, the one or more base edits target translation initiation or elongation by introducing mutations in the mRNA's 5′ untranslated (5′UTR), 3′ untranslated region (3′UTR), or within the coding sequence, affecting translation initiation or elongation and resulting in decreased production of a gene product.

In one embodiment, a RNA base editing system is used to introduce one or more base edits to one or more RNA molecules transcribed from one or more genes containing one or more targets of the randomized guide molecules, such that expression or activity of the gene product is increased. In one embodiment, the one or more base edits are used to change suboptimal codons to more frequently used codons (while maintaining the same amino acid sequence) in the coding region of the mRNA, leading to improved translation efficiency and gene product production. In one embodiment, the one or more base edits remove inhibitor sequences in the mRNA. Some mRNA contain regulatory elements, such as uORFs and IRESs, that inhibit gene expression. The one or more base edits may be used to disrupt or remove these inhibitor sequences thereby increasing gene product production. In one embodiment, the one or more base edits may be used to modify regulatory elements within the mRNA, such as mRNA stability elements, microRNA binding sites, or RNA binding protein sites may enhance mRNA stability, translation efficiency, or prevent degradation, leading to increased gene expression. In one embodiment, the one or more base edits may introduce one or more mutations in the 5′ UTR or the 3′ UTR that enhance translation initiation or elongation, resulting in increased gene product production. In one embodiment, the one or more base edits may be used to introduce specific point mutations or modifications within the coding region of the mRNA using RNA based editors that can potentially improve the catalytic activity, binding affinity, or other functional properties of the gene product. While this approach may not directly increase mRNA translation it can result in an overall increase of the functional output of the gene product.

### ARCUS Base Editing

In one example embodiment, a target polynucleotide (e.g., a polynucleotide containing one or more targets of the randomized guide molecules) is modified with an ARCUS base editing system. Exemplary methods for using ARCUS can be found in U.S. Pat. No. 10,851,358, U.S. Patent Application Publication No. 2020-0239544, and WIPO Publication No. 2020/206231, which are incorporated herein by reference in their entirety. In some embodiments, the target polynucleotide is a polynucleotide in a gene, genome, transcript, or transcriptome.

In certain embodiments, the ARCUS base editing system comprises a nuclease, derived from I-CreI endonuclease (hereinafter, an “ARC nuclease”) with a recognition sequence for the one or more genes containing one or more targets of the randomized guide molecules and/or transcription factors (or polynucleotides encoding the same) containing one or more targets of the randomized guide molecules. In certain embodiments, the nuclease is a homing endonuclease or meganuclease as described in the section titled “Meganucleases”. In certain embodiments, the ARC nuclease is an engineered meganuclease prepared to recognize a target gene or transcription factor, or a region of a target gene or transcription factor. In certain embodiments, the ARC nuclease comprises a single-component protein containing both a site-specific DNA recognition interface and endonuclease activity. The combination of both substrate-recognition and catalytic motifs into a single protein have been shown to allow for both viral and non-viral delivery modalities (see, e.g., Gorsuch et al. (2022). Targeting the hepatitis B cccdna with a sequence-specific arcus nuclease to eliminate hepatitis B virus in vivo. Molecular Therapy, 30(9), 2909-2922. doi.org/10.1016/j.ymthe.2022.05.013).

In one example embodiment, the ARC nuclease is configured to decrease the expression of the one or more genes or transcription factors containing one or more targets of the randomized guide molecules or increase the expression of the one or more genes or transcription factors. In an example embodiment, the ARC nuclease scans a region of a target gene for the target site. For example, the ARCUS nuclease looks for a polynucleotide or region within one or more open reading frames of the one or more genes or transcription factors containing one or more targets of the randomized guide molecules. After binding to the target site, the DNA sequence is cut, creating a sticky 4-base 3′ overhang wherein the cut target site is repaired via homology-directed repair (HDR) or non-homologous end-joining (NHEJ). Non-homologous end-joining can result in insertions, deletions, substitutions, or otherwise a frameshift mutation that can interfere with gene expression. In one example embodiment, the interference with gene expression results in the decreased expression of the one or more genes or transcription factors from containing one or more targets of the randomized guide molecules or increased expression of the one or more genes or transcription factors containing one or more targets of the randomized guide molecules. HDR or NHEJ methods for repaired joining, and optionally, specific templates that could be utilized, are described in the respective sections titled “HDR Template Based Editing” and “NHEJ-Based Editing”. In certain embodiments, an additional template may prevent off-site insertions or deletions.

### Other Programmable Nuclease Systems

In example embodiments, the programable nuclease may comprise of a prime editor, CRISPR associated transposon system, and/or non-LTR retrotransposon.

### Prime Editors

The present disclosure also provides for a prime editing system to either decrease expression of one or more genes or transcription factors containing one or more targets of the randomized guide molecules or increase the expression of one or more genes or transcription factors containing one or more targets of the randomized guide molecules. Prime editing (PE) systems comprise a programable nuclease (e.g., Cas or other programmable nuclease described herein), most often a nickase, linked to a reverse transcriptase domain and a guide molecule (prime editing guide pegRNA), which comprises a target-specific spacer, a primer binding site, and an RT template. See e.g., Anzalone et al. 2019. Nature. 576: 149-157; and International Patent Application Publication No. WO 2022/150790A2. In some embodiments, the prime editing guide molecule can specify both the target polynucleotide information (e.g., sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g., a prime editing guide molecule or peg guide molecule) directly into the target site in the target polynucleotide. See e.g., Anzalone et al. 2019. Nature. 576: 149-157, particularly at FIGS. 1b, 1c, related discussion, and Supplementary discussion.

### Recombinase-Mediated Modifications

Prime editing systems can also be further combined with site-specific recombinases, such as integrases, to facilitate even larger insertions, substitutions and deletions. See e.g., WO 2021/138469; Anzalone AV, Gao XD, Podracky C J, et al. Programmable deletion, replacement, integration and inversion of large DNA sequences with twin prime editing. Nat Biotechnol. 2022; 40(5):731-740; Yarnall et al., NatBiotechnol (2022). doi.org/10.1038/s41587-022-01527-4, which is incorporated by reference as if expressed in its entirety herein. The prime editing system is used to insert a recombinase recognition site at the desire site of modification and an integrase facilitates the insertion of a donor sequence from a donor template. “Uni-directional recombinases” or “integrases” refer to recombinase enzymes whose recognition sites are destroyed after the recombination has taken place. The term “integrase” refers to a type of recombinase. In other words, the sequence recognized by the recombinase is changed into one that is not recognized by the recombinase upon recombination. As a result, once a sequence is subjected to recombination by the uni-directional recombinase, the continued presence of the recombinase cannot reverse the previous recombination event.

Typically, two different sites are involved (in regard to recombination termed “complementary sites”), one present in the target nucleic acid (e.g., a chromosome or episome of a eukaryote) and another on the nucleic acid that is to be integrated at the target recombination site. The terms “attB” and “attP,” which refer to attachment (or recombination) sites originally from a bacterial target (attachment site of bacteria) and a phage donor (attachment site of phage), respectively, are used herein although recombination sites for particular enzymes may have different names. The two attachment sites can share as little sequence identity as a few base pairs. The recombination sites typically include left and right arms separated by a core or spacer region. Thus, an attB recombination site consists of BOB′, where B and B′ are the left and right arms, respectively, and O is the core region. Similarly, attP is POP′, where P and P′ are the arms and O is again the core region. Upon recombination between the attB and attP sites, and concomitant integration of a nucleic acid at the target, the recombination sites that flank the integrated DNA are referred to as “attL” and “attR.” The attL and attR sites, using the terminology above, thus consist of BOP‘ and POB’, respectively. In some representations herein, the “O” is omitted and attB and attP, for example, are designated as BB‘ and PP’, respectively.

In example embodiments, the recombinase is a serine integrase. In example embodiments, serine integrases specifically recombine when recognizing the two attachment sites specific for the integrase. In example embodiments, the heterologous sites are referred to as attP and attB, however, these terms refer to the specific sequences recognized by the specific integrase and do not refer to a single consensus sequence. Serine integrases mediate site-specific recombination between short recognition sites located in phage genomes and bacterial chromosomes, respectively, the attachment site of phage (attP) and attachment site of bacteria (attB) (i.e., the target sites of the integrase), to form the hybrid attachment sites attL and attR. Unlike Cre and Flp recombinases that catalyze reversible site-specific recombination reactions, serine integrases are unidirectional and catalyze only attP and attB recombination without RDF or Xis accessory proteins. Thus, in the absence of any accessory factors, integrase is unidirectional. In addition, DNA substrates identified by serine integrases (attP and attB) are relatively short (30-50 bp) and have a minimal length of approximately 34-40 base pairs (bp) (Groth A C et al., Proc. Natl. Acad. Sci. USA 97, 5995-6000 (2000)). The compatibility of distinct DNA topological structures is also quite different from recognition of DNA by Hin recombinase or Tn3 resolvase. Serine integrases recognize DNA substrates specifically, not at random, but can facilitate recombination at sequences with partial identity with wild-type recombination sites, termed pseudo attachment sites (either pseudo attP or pseudo attB). A “pseudo-recombination site” is a DNA sequence recognized by a recombinase enzyme such that the recognition site differs in one or more base pairs from the wild-type recombinase recognition sequence and/or is present as an endogenous sequence in a genome that differs from the genome where the wild-type recognition sequence for the recombinase resides. “Pseudo attP site” or “pseudo attB site” refer to pseudo sites that are similar to wild-type phage or bacterial attachment site sequences, respectively, for phage integrase enzymes. “Pseudo att site” is a more general term that can refer to either a pseudo attP site or a pseudo attB site. Specific attB and attP sequences for use include all wildtype sequences as well as pseudo attB and attP sequences.

Recombination sites used in the present methods include those recognized by unidirectional, site-directed recombinases (e.g., integrases). Non-limiting examples of serine integrases and recombination sites applicable herein include C31 integrase, Bxbl, BT1 integrase, A118, TP901-1, and R4 and the corresponding recombination sites for each (see, e.g., Groth, A. C. and Calos, M. P. (2004) J. Mol. Biol. 335, 667-678; Lei, et al., FEBS Lett. 2018 April; 592(8):1389-1399; Singh, et al., Attachment Site Selection and Identity in Bxbl Serine Integrase-Mediated Site-Specific Recombination, PLoS Genet. 2013 May; 9(5):e1003490; and Gupta, et al., Nucleic Acids Res. 2007 May; 35(10): 3407-3419). Additional serine recombinases and recombination sites may be any of those disclosed in U.S. Patent Application Publication No. 2018/0346934A1 and U.S. Patent Application Publication No. 2010/0190178. In certain embodiments, a functional domain of the serine integrase is used.

In one example embodiment, the system can be used to insert or replace a sequence into one or more target genes. In example embodiments, the insertion or replacement results in an inactive target gene or less active form of the target gene. In one example embodiment, the system is used to replace all or a portion of the entire target gene. In one example embodiment, the system is used to replace all or a portion of an enhancer controlling the target gene expression.

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### Example Prime Editing Modifications for Decreasing or Increasing Expression of Target Genes

Prime Editing systems may be used to introduce insertions, deletions, or substitutions (modifications) that control expression of the one or more genes containing one or more targets of the randomized guide molecules. The modifications may be made in a non-coding region that controls expression of the one or more target genes (e.g., genes containing one or more targets of the randomized guide molecules), in a coding region encoding a gene expression product (e.g., a polypeptide), or both. Example modifications are described in further detail below. As a threshold matter, Prime Editing systems are capable of making all 4 base edits (A, T, C, G) and thus can be used to make all of the same DNA base edits described above in the Base Editor section. The following examples focus on additional insertions, deletions and substitutions that may be made using prime editors beyond single base edits using standard prime editors (PE), twinPE, or PE/twinPE in combination with a recombinase, which for purposes of the following example modification sections will be referred to collectively as prime editors.

### Prime Editing Modifications That Decrease Expression By Targeting Non-Coding Regions

In one example embodiment, the prime editing system is configured to introduce a deletion, insertion, or mutation in one or more non-coding regions controlling expression of one or more genes containing one or more targets of the randomized guide molecules such that the expression of the one or more genes is reduced. In one embodiment, the one or more modifications remove, modify, or disrupt an enhancer such that binding of transcription factors or other regulatory proteins controlling expression is disrupted, thereby reducing transcription initiation and gene expression. In one embodiment, the one or more modifications remove or disrupt an existing promoter or replace the existing promoter with a weakened promoter such that the binding of transcription factors and/or RNA polymerase binding are blocked or reduced. In one embodiment, the prime editing system is configured to introduce a silencer element into the non-coding region leading to the recruitment of transcriptional repressors that block or decrease gene expression. In one embodiment, the prime editor is configured to modify or replace an existing silencer element such that the silencing function of the silencer element is increased relative to an unmodified silencer sequence. In another embodiment, the prime editor is configured to disrupt or replace one or more insulator sequences such that nearby silencer elements or repressive chromatin structures can decrease gene expression.

### Prime Editing Modifications That Increase Expression by Targeting Non-Coding Regions

In another embodiment, the prime editor is configured to introduce one or more enhancer regions controlling expression of one or more genes containing one or more targets of the randomized guide molecules such that binding of transcription factors or other regulatory proteins is increased or strengthened and gene expression is increased. In another embodiment, the prime editor is configured to introduce one or more modifications in one or more promoter regions controlling expression of one or more genes containing one or more targets of the randomized guide molecules such that binding of transcription factors or RNA polymerase is increased or strengthened and gene expression is increased. In another embodiment, the prime editor is configured to introduce insertions/deletions/substitutions that disrupt or remove one or more silence elements, thereby preventing binding of transcriptional repressors and increasing gene expression. In another embodiment, the prime editor is configured to introduce or strengthen insulator sequences, thereby reducing the influence of nearby silencer elements or repressive chromatin structures such that gene expression is increased.

### Prime Editing Modifications That Decrease Expression By Targeting Coding Regions

In one embodiment, the prime editor is configured such that one or more modifications (e.g., insertions, deletions, substitutions) are made in a coding region of the one or more genes containing one or more targets of the randomized guide molecules such that expression of the one or more genes is reduced. In one embodiment, the one or more modifications result in a frame-shift mutation leading to introduction of a premature stop codon and the production of a non-functional, truncated gene product or the triggering of nonsense-mediated mRNA decay (NMD), thereby resulting in reduced expression or gene product activity. In another embodiment, the one or more modifications result in introduction of a premature stop codon within the coding region resulting in production of truncated non-functional proteins or the triggering of NMD and thereby resulting in reduced gene expression or gene product activity. In another embodiment, the one or more modifications target specific functional domains within the coding region to create insertions, deletions, or mutations that impair the function of the gene product. While this approach may not directly decrease gene expression, it can lead to the production of non-functional proteins, effectively resulting in a loss-of-function effect. In another embodiment, the one or more modifications introduce mutations in the coding region at exon-intron boundaries or splice sites leading to aberrant splicing, production of non-functional proteins or triggering NMD and thereby reducing gene expression or activity of a resulting gene product. In another embodiment, the one or more modifications may target regulatory elements within the coding regions that affect gene expression, such as internal ribosome entry sites (IRES). One or more modifications may be made at these regulatory elements to reduce gene expression. In another embodiment, the one or more modifications may introduce, change, or remove a sequence encoding a post-translation modification (PTM) site in the expressed gene product. Post-translational modification, such as phosphorylation, glycosylation, or ubiquitination, play an essential role in regulating protein function, stability and localization. Post-translation modification may be both necessary to inhibit a protein's functions or to activate a protein's function. Accordingly, modifications that introduce inhibitory PTMs or remove activating PTMs may be made to decrease protein function, stability, and/or degradation.

### Prime Editing Modifications That Increase Expression By Targeting Coding Regions

In one embodiment, the programmable nuclease and donor template are configured such that one or more modifications (e.g., insertions, deletions, substitutions) are made in a coding region of the one or more genes containing one or more targets of the randomized guide molecules such that expression of the one or more genes is increased. In one embodiment, the one or more modifications comprise removing inhibitors sequences, such as IRESs or upstream open reading frames (uORFs), which can negatively affect expression. In one embodiment, the one or more modifications may comprise introducing specific mutations or modifications within the coding region that can potentially improve protein stability, folding, or resistance to degradation. While this does not directly increase gene expression, it can lead to higher protein levels and enhanced function. In one embodiment, the modification may comprise removal or disruption of a sequence encoding an inhibitory PTM site, removal or disruption of one or more ubiquitination sites, or introduction of PTM sites that stabilize or enhance protein function. In one embodiment, the one or more modification may comprise mutations or modifications within the coding region that improve catalytic activity, binding affinity, or other functional properties of the protein. This approach does not directly increase gene expression but can result in an overall increase in the functional output of the gene product. In another embodiment, prime editing and/or twinPE are used in combination with a recombinase to insert an additional functional copy of the one or more genes containing one or more targets of the randomized guide molecules.

### CRISPR Associated Transposase (CAST) Systems

In one example embodiment, the nucleic acid modifying agent is a CRISPR associated transposase system (CAST). In one example embodiment, a CAST system is used to edit a plurality of genomic loci. A CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class 1 or Class 2 CAST systems. Example CAST systems are disclosed in Klompe et al. “Transposon-encoded CRISPR-Cas systems direct RNA-guided DNA integration,” Nature, 571:219-225 (2019); Saito et al. “Dual modes of CRISPR-associated transposon homing” Cell, 184(9):2441-2453 (2021); Cameron et al. “Harnessing Type 1 CRISPR-Cas systems for human genome engineering,” Nat Biotechno, 37:1471-1477 (2019); Halpin-Healy et al. “Structural basis of DNA targeting by transposon-encoded CRISPR-Cas systems” Nature, 577:271-274 (2020), Klompe et al. “Evolutionary and mechanistic diversity of Type I-F CRISPR-associated transposons,” Mol Cell, 82:616-628 (2022) An example Class 2 system is described herein and in Strecker et al. “RNA-guided DNA insertion with CRISPR-associated transposase,” Science, 365(6448):48-53 (2019)), and PCT/US2019/066835, which are incorporated herein by reference in their entirety.

The systems herein may comprise one or more components of a transposon and/or one or more transposases. The transposases in the systems herein may be CRISPR-associated transposases (also used interchangeably with Cas-associated transposases, CRISPR-associated transposase proteins herein) or functional fragments thereof. CRISPR-associated transposases may include any transposases that can be directed to or recruited to a region of a target polynucleotide by sequence-specific binding of a CRISPR-Cas complex. CRISPR-associated transposases may include any transposases that associate (e.g., form a complex) with one or more components in a CRISPR-Cas system, e.g., Cas protein, guide molecule etc.). In certain example embodiments, CRISPR-associated transposases may be fused or tethered (e.g., by a linker) to one or more components in a CRISPR-Cas system, e.g., Cas protein, guide molecule etc.).

The term “transposon”, as used herein, refers to a polynucleotide (or nucleic acid segment), which may be recognized by a transposase or an integrase enzyme and which is a component of a functional nucleic acid-protein complex (e.g., a transpososome, or transposon complex) capable of transposition. Transposons employ a variety of regulatory mechanisms to maintain transposition at a low frequency and sometimes coordinate transposition with various cell processes. Some prokaryotic transposons can also mobilize functions that benefit the host or otherwise help maintain the element.

The term “transposase” as used herein refers to an enzyme, which is a component of a functional nucleic acid-protein complex capable of transposition and which mediates transposition. The transposase may comprise a single protein or comprise multiple protein sub-units. A transposase may be an enzyme capable of forming a functional complex with a transposon end or transposon end sequences. The term “transposase” may also refer in certain embodiments to integrases. The expression “transposition reaction” used herein refers to a reaction wherein a transposase inserts a donor polynucleotide sequence in or adjacent to an insertion site on a target polynucleotide. The insertion site may contain a sequence or secondary structure recognized by the transposase and/or an insertion motif sequence where the transposase cuts or creates staggered breaks in the target polynucleotide into which the donor polynucleotide sequence may be inserted. Exemplary components in a transposition reaction include a transposon, comprising the donor polynucleotide sequence to be inserted, and a transposase or an integrase enzyme. The term “transposon end sequence” as used herein refers to the nucleotide sequences at the distal ends of a transposon. The transposon end sequences may be responsible for identifying the donor polynucleotide for transposition. The transposon end sequences may be the DNA sequences the transpose enzyme uses in order to form a transpososome complex and to perform a transposition reaction.

In some embodiments, the system comprises one or more Tn7 transposase polypeptides. In some embodiments, three transposon-encoded proteins form the core transposition machinery of Tn7: a heteromeric transposase (TnsA and TnsB) and a regulator protein (TnsC). In addition to the core TnsABC transposition proteins, Tn7 elements encode dedicated target site-selection proteins, TnsD and TnsE. In conjunction with TnsABC, the sequence-specific DNA-binding protein TnsD directs transposition into a conserved site referred to as the “Tn7 attachment site,” attTn7 via its C-terminal that binds directly with DNA. TnsD (e.g. TnsD1 and TnsD2) is a member of a large family of proteins that also includes TniQ (e.g. TniQ1 and TniQ2), a protein found in other types of bacterial transposons. TniQ has been shown to target transposition into resolution sites of plasmids. TniQ works with Cascade/Cas12k (CAST) for RNA guided transposition. TniQ is a shorter version of TnsD comprising around 300 amino acids. TniQ also comprises a N-terminal similar to that of TnsD but lacks the corresponding C-terminal. Therefore, TniQ interacts with the Cascade to bind DNA. The addition of a TnsD C-terminal to a TniQ would amount to a TnsD. As used herein, a TniQ transposase may be a TnsD transposase. In some examples, the Tn7 comprises a transposase that has the activities of typical TnsA and TnsB. In an example embodiment, the transposase that has the activities of typical TnsA and TnsB is a fusion protein and may also be referred to as TnsAB. In some examples, the transposase is not a fusion protein of typical TnsA and TnsB. An example of the transposase is TnsA in IB20. Examples of Tn7 transposase polypeptides include but are not limited to TnsA, TnsB, TnsC, TniQ, TnsD, and TnsE.

As used herein, a right end sequence element or a left end sequence element are made in reference to an example Tn7 transposon. The general structure of the left end (LE) and right end (RE) sequence elements of canonical Tn7 is established. Tn7 ends comprise a series of 22-bp TnsB-binding sites. Flanking the most distal TnsB-binding sites is an 8-bp terminal sequence ending with 5′-TGT-3′/3′-ACA-5′. The right end of Tn7 contains four overlapping TnsB-binding sites in the ~90-bp right end element. The left end contains three TnsB-binding sites dispersed in the ~150-bp left end of the element. The number and distribution of TnsB-binding sites can vary among Tn7-like elements. End sequences of Tn7-related elements can be determined by identifying the directly repeated 5-bp target site duplication, the terminal 8-bp sequence, and 22-bp TnsB-binding sites (Peters J E et al., Proc. Nat. Acad. Sci. 114(35) E7358-E7366 (2017)). Example Tn7 elements, including right end sequence element and left end sequence element include those described in Parks AR, Plasmid, 2009 January; 61(1):1-14.

As used herein, Tn7 transposons and transposases include Tn7-like transposons and transposases. For further guidance on CAST nucleic acid modifying agent see U.S. Pat. No. 11,384,344 B2, incorporated herein by reference in its entirety.

### TnpB Retrotransposon Systems

In one example embodiment, the nucleic acid modifying agent is a TnpB, one or more nucleic acid components, and one or more components of a retrotransposon, e.g., a non-LTR retrotransposon. The one or more components of a retrotransposon include a retrotransposon protein and retrotransposon RNA. The systems and compositions may be used to insert a donor polynucleotide into a target polynucleotide. The systems and compositions may further comprise a donor polynucleotide.

In example embodiments, the present disclosure provides an engineered, non-naturally occurring composition comprising: a TnpB polypeptide, a non-LTR retrotransposon protein associated with or otherwise capable of forming a complex with the TnpB polypeptide; a single nucleic acid component capable of forming a complex with the TnpB polypeptide and directing site-specific binding to a target sequence of a target polynucleotide. The composition may further comprise a donor construct comprising a donor polynucleotide for insertion in to the target polynucleotide and located between two binding elements capable of forming a complex with the non-LTR retrotransposon protein. In some cases, the TnpB polypeptide is engineered to have nickase activity.

In some examples, the TnpB polypeptide is fused to the N-terminus of the non-LTR retrotransposon protein. In some examples, the TnpB polypeptide is fused to the C-terminus of the non-LTR retrotransposon protein.

The nucleic acid component molecule s may direct the fusion protein to a target sequence 5′ of the targeted insertion site, and wherein the TnpB polypeptide generates a double-strand break at the targeted insertion site. The nucleic acid component molecule s may direct the fusion protein to a target sequence 3′ of the targeted insertion site, and wherein the TnpB polypeptide generates a double-strand break at the targeted insertion site.

The donor polynucleotide may further comprise a polymerase processing element to facilitate 3′ end processing of the donor polynucleotide sequence. The polymerase may be a DNA polymerase, e.g., DNA polymerase I. In some examples, the polymerase may be an RNA polymerase.

In some examples, the donor polynucleotide further comprises a homology region to the target sequence on the 5′ end of the donor construct, the 3′ end of the donor construct, or both. In some examples, the homology region is from 1 to 50, from 5 to 30, from 8 to 25, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 base pairs in length.

Native or wild-type non-LTR retrotransposons encode the protein machinery necessary for their self-mobilization. The non-LTR retrotransposon element comprises a DNA element integrated into a host genome. This DNA element may encode one or two open reading frames (ORFs). For example, the R2 element of Bombyx mori encodes a single ORF containing reverse transcriptase (RT) activity and a restriction enzyme-like (REL) domain. L1 elements encode two ORFs, ORF1 and ORF2. ORF1 contains a leucine zipper domain involved in protein-protein interactions and a C-terminal nucleic acid binding domain. ORF2 has a N-terminal apurinic/apyrimidinic endonuclease (APE), a central RT domain, and a C-terminal cysteine histidine rich domain. An example replicative cycle of a non-LTR retrotransposon may comprise transcription of the full-length retrotransposon element to generate an mRNA active element (retrotransposon RNA). The active element mRNA is translated to generate the encoded retrotransposon proteins or polypeptides. A ribonucleoprotein complex comprising the active element and retrotransposon protein or polypeptide is formed and this RNP facilitates integration of the active element into the genome. The RNA-transposase complex nicks the genome. The 3′ end of the nicked DNA serves as a primer to allow the reverse transcription of the transposon RNA into cDNA. Fourth, the transposase proteins integrate the cDNA into the genome.

Elements of these systems may be engineered to work within the context disclosed herein. For example, a non-LTR retrotransposon polypeptide may be fused to a site-specific nuclease. The binding elements that allow a non-LTR retrotransposon polypeptide to bind to the native retrotransposon DNA element, may be engineered into a donor construct to facilitate entry of a donor polynucleotide sequence into a target polypeptide.

In the present disclosure, the protein component of the non-LTR retrotransposon may be connected to or otherwise engineered to form a complex with a site-specific nuclease, e.g., TnpB polypeptide. The retrotransposon RNA may be engineered to encode a donor polynucleotide sequence. Thus, in certain example embodiments, the TnpB polypeptide, via formation of a TnpB polypeptide complex with a nucleic acid component molecule sequence, directs the retrotransposon complex (e.g., the retrotransposon polypeptide(s) and retrotransposon RNA to a target sequence in a target polynucleotide, where the retrotransposon RNP complex facilitates integration of the donor polynucleotide sequence into the target polynucleotide. Accordingly, the one or more non-LTR retrotransposon components may comprise retrotransposon polypeptides, or function domains thereof, that facilitate binding of the retrotransposon RNA, reverse transcription of the retrotransposon RNA into cDNA, and/or integration of the donor polynucleotide into the target polynucleotide, as well as retrotransposon RNA elements modified to encode the donor polynucleotide sequence.

Examples of non-LTR retrotransposons include CRE, R2, R4, L1, RTE, Tad, R1, LOA, I, Jockey, CR1. In one example, the non-LTR retrotransposon is R2. In another example, the non-LTR retrotransposon is L1. Examples of non-LTR retrotransposons may include those described in Christensen S M et al., RNA from the 5′ end of the R2 retrotransposon controls R2 protein binding to and cleavage of its DNA target site, Proc Natl Acad Sci USA. 2006 Nov. 21; 103(47):17602-7; Eickbush T H et al, Integration, Regulation, and Long-Term Stability of R2 Retrotransposons, Microbiol Spectr. 2015 April; 3(2):MDNA3-0011-2014. doi: 10.1128/microbiolspec. MDNA3-0011-2014; Han J S, Non-long terminal repeat (non-LTR) retrotransposons: mechanisms, recent developments, and unanswered questions, Mob DNA. 2010 May 12; 1(1):15. doi: 10.1186/1759-8753-1-15; Malik H S et al., The age and evolution of non-LTR retrotransposable elements, Mol Biol Evol. 1999 June; 16(6):793-805, which are incorporated by reference herein in their entireties. Examples of the non-LTR retrotransposon polypeptides also include R2 from Clonorchis sinensis, or Zonotrichia albicollis.

A non-LTR retrotransposon may comprise multiple retrotransposon polypeptides or polynucleotides encoding same. In one embodiment, the retrotransposon polypeptides may form a complex. For example, a non-LTR retrotransposon is a dimer, e.g., comprising two retrotransposon polypeptides forming a dimer. The dimer subunits may be connected or form a tandem fusion. A TnpB polypeptide may be associated with (e.g., connected to) one or more subunits of such complex. In some examples, the non-LTR retrotransposon is a dimer of two retrotransposon polypeptides; one of the retrotransposon polypeptides comprises nuclease or nickase activity and is connected with a TnpB polypeptide.

The retrotransposon polypeptides may comprise one or more modifications to, for example, enhance specificity or efficiency of donor polynucleotide recognition, target-primed template recognition (TPTR). The retrotransposon polypeptides may also comprise one or more truncations or excisions to remove domains or regions of wild-type protein to arrive at a minimal polypeptide that retain donor polynucleotide recognition and TPTR. In some example embodiments, the native endonuclease activity may be mutated to eliminate endonuclease activity.

In certain example embodiments, the modifications or truncations of the non-LTR retrotransposon peptide may be in a zinc finger region, a Myb region, a basic region, a reverse transcriptase domain, a cysteine-histidine rich motif, or an endonuclease domain.

A non-LTR retrotransposon may comprise polynucleotide encoding one or more retrotransposon RNA molecules. The polynucleotide may comprise one or more regulatory elements. The regulatory elements may be promoters. The regulatory elements and promoters on the polynucleotides include those described throughout this application. For example, the polynucleotide may comprise a pol2 promoter, a pol3 promoter, or a T7 promoter.

In some cases, the polynucleotide encodes a retrotransposon RNA with at least a portion of its sequence complementary to a target sequence. For example, the 3′ end of the retrotransposon RNA may be complementary to a target sequence. The RNA may be complementary to a portion of a nicked target sequence. In one embodiment, a retrotransposon RNA may comprise one or more donor polynucleotides. In certain cases, a retrotransposon RNA may encode one or more donor polynucleotides.

A retrotransposon RNA may be capable of binding to a retrotransposon polypeptide. Such retrotransposon RNA may comprise one or more elements for binding to the retrotransposon polypeptide. Examples of binding elements include hairpin structures, pseudoknots (e.g., a nucleic acid secondary structure containing at least two stem-loop structures in which half of one stem is intercalated between the two halves of another stem), stem loops, and bulges (e.g., unpaired stretches of nucleotides located within one strand of a nucleic acid duplex). In certain examples, the retrotransposon RNA comprises one or more hairpin structures. In some examples, the retrotransposon RNA comprises one or more pseudoknots. In certain examples, a retrotransposon RNA comprises a sequence encoding a donor polynucleotide and one or more binding elements for forming a complex with the retrotransposon polypeptide. The binding elements may be located on the 5′ end or the 3′ end.

In one embodiment, a retrotransposon RNA comprises a region capable of hybridizing with an overhang of a target polynucleotide at the target site. The overhang may be a stretch of single-stranded DNA. The overhang may function as a primer for reverse transcription of at least a portion of the retrotransposon RNA to a cDNA. In some cases, a region of the cDNA may be capable of hybridizing a second overhang of the target polynucleotide. The second overhang may function as a primer for the synthesis of a second strand to generate a double-stranded cDNA. The cDNA may comprise a donor polynucleotide sequence. The two overhangs may be from different strands of the target polynucleotide.

### Additional Prime Systems

Prime editing systems can also be used in tandem such that, the two pegRNAs template the synthesis of complementary DNA flaps on opposing strands of genomic DNA, which replace the endogenous DNA sequence between the PE-induced nick sites. See, e.g., Anzalone AV, Gao XD, Podracky C J, et al. Programmable deletion, replacement, integration and inversion of large DNA sequences with twin prime editing. Nat Biotechnol. 2022; 40(5):731-740. Thus, use of two pegRNAs allows for larger insertions or deletions because of the two overlapping 3′ flaps created by the two nicked sites. In one example embodiment, the system can be used to insert or replace a sequence into one or more target genes, such as those gene(s) containing one or more targets of the randomized guide molecules. In example embodiments, the insertion or replacement results in an inactive target gene or less active form of the target gene, such as those gene(s) containing one or more targets of the randomized guide molecules. In one example embodiment, the system is used to replace all or a portion of the entire target gene. In one example embodiment, the system is used to replace all or a portion of an enhancer controlling the target gene expression. twinPE systems can also be further combined with site-specific recombinases, such as integrases, to facilitate even larger insertions, substitutions and deletions as described above.

In example embodiments, the prime editing system is capable of simultaneous editing of both strands of a target double-stranded nucleotide sequence. For example, to accomplish simultaneous editing of both strands of a target double-stranded nucleotide sequence, a prime editing system may comprise a first and second prime editor complex as described in International Patent Publication No. WO 2021/226558 A8, incorporated herein by reference.

### Delivery of Guide Molecules and Programmable Nucleases

The delivery systems may comprise one or more delivery vehicles. The delivery vehicles may deliver the cargo into cells, tissues, organs, or organisms (e.g., animals or plants). The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses (e.g., virus particles), non-viral vehicles (e.g., mRNA, RNP), and other delivery reagents described herein.

In example embodiments, guide molecules and programmable nucleases for pooled multi-site edits are delivered to target cells using one or more vectors. As used herein, the delivery of nucleic acids into eukaryotic cells is called transfection. In example embodiments, multiple guide molecules are introduced simultaneously to a host cell for one-step encoding of information by editing at multiple genomic loci. In example embodiments, the method further comprises, before step (a), preparing a screening pool of guide molecule vectors comprising the set of guide molecules, wherein the set of guide molecules target a plurality of target sites in a cell genome and are configured to introduce the set of edits. In example embodiments, guide molecules that show robust editing in pools and demonstrate simultaneous multi-site editing across >100 genomic sites are introduced by one or more vectors. Applicants demonstrated that individual stem cells with more than two dozen edits can be obtained with minimal screening using guide molecules selected for robust editing when introduced to cells within a pool of guide molecules. Applicants observed that editing rate decreases with increasing transfected guide molecule batch size, but that this could be overcome using the selected for guide molecules. The guide molecules in these experiments were transfected into host cells on separate plasmids to avoid recombination within the plasmids and to allow for easily changing the combination of guide molecules used for cryptography. All of the guide molecules were expressed from the same plasmid vector and each transfected cell received all of the vectors in the pool. Therefore, the guide molecule sequences were responsible for the differences in editing rates between guide molecules in experiments using pooled transfection of multiple guide molecules. Thus, any vector capable of transferring the guide molecules to host cells can be used with the selected guide molecules to perform pooled multi-site base editing. In example embodiments, the choice of vector depends on the host cell, such as primary cells, stem cells, or immortalized tissue culture cells. For example, primary cells may be more difficult to transfect than tissue culture cells.

In example embodiments, the guide molecules are introduced on separate vectors, each vector encoding a single guide molecule. In example embodiments, a specific set of selected guide molecules is encoded for on a single vector. In example embodiments, a single vector can encode any of 1 to 20 guide molecules. In example embodiments, multiple vectors are used in pooled multi-site base editing. In example embodiments, a vector with more than one guide molecule is designed to have decreased recombination between homologous sequences (e.g., variation between guide molecule constant sequences).

In general, and throughout this specification, the term “vector” refers to a nucleic acid construct or carrier molecule capable of transporting a nucleic acid to which it has been linked to a host cell. Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses). A “viral vector” is also defined as a recombinantly produced virus or viral particle that comprises a polynucleotide to be delivered into a host cell, either in vivo, ex vivo or in vitro. Viral vectors also include polynucleotides carried by a virus for transfection into a host cell. Viral vectors can be used to transfer DNA or RNA into cells in a process called transduction. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Vectors for and that result in expression in a eukaryotic cell can be referred to herein as “eukaryotic expression vectors.” Common expression vectors of utility in recombinant DNA techniques are often in the form of plasmids. There are no limitations regarding the type of vector that can be used. The vector can be a cloning vector, suitable for propagation and for obtaining polynucleotides, gene constructs or expression vectors incorporated into several heterologous organisms. Suitable vectors include eukaryotic expression vectors based on viral vectors (e.g., adenoviruses, adeno-associated viruses, retroviruses and lentiviruses, and herpes viruses), as well as non-viral vectors such as plasmids. In example embodiments, the vector can be a viral vector. In example embodiments, the vector can be a liposome. In example embodiments, the vector can be a nanoparticle. In example embodiments, the vector can be a Ribonucleoprotein complex (RNP). In the present specification, “plasmid” and “vector” can be used interchangeably as the plasmid is the most commonly used form of vector. However, the methods and compositions described herein can include such other forms of expression vectors, such as viral vectors replication defective retroviruses, lentiviruses, adenoviruses and adeno-associated viruses), which serve equivalent functions. Viral vectors may, for example, also include vectors based on HIV, SV40, EBV, HSV or BPV. Despite success with lentiviral delivery, Hendel et al, (Nature Biotechnology 33, 985-989 (2015) doi:10.1038/nbt.3290) showed the efficiency of editing human T-cells with chemically modified RNA, and direct RNA delivery to T-cells via electroporation. In some embodiments, one or more of the viral or plasmid vectors may be delivered via liposomes, nanoparticles, exosomes, microvesicles, electroporation, or a gene-gun.

In example embodiments, base editing systems may be delivered as a pre-complex Ribonucleoprotein complex (RNP). Systems for delivering RNPs include the protein delivery systems: virus like particles; cell-penetrating peptides; and nanocarriers. In example embodiments, the Cas polypeptide and guide molecule may also be delivered as a pre-formed ribonucleoprotein complex (RNP). Delivery methods for delivery RNPs include virus like particles, cell-penetrating peptides, and nanocarriers. In particular embodiments, pre-complexed guide RNA and CRISPR effector protein, (optionally, adenosine deaminase fused to a CRISPR protein or an adaptor) are delivered as a ribonucleoprotein (RNP). RNPs have the advantage that they lead to rapid editing effects even more so than the RNA method because this process avoids the need for transcription. An important advantage is that both RNP delivery is transient, reducing off-target effects and toxicity issues. Efficient genome editing in different cell types has been observed by Kim et al. ((2014, Genome Res. 24(6):1012-9)), Paix et al. (2015, Genetics 204(1):47-54), Chu et al. (2016, BMC Biotechnol. 16:4), and Wang et al. (2013, Cell. 9; 153(4):910-8). In example embodiments, the ribonucleoprotein is delivered by way of a polypeptide-based shuttle agent as described in WO 2016/161516. WO 2016/161516 describes efficient transduction of polypeptide cargos using synthetic peptides comprising an endosome leakage domain (ELD) operably linked to a cell penetrating domain (CPD), to a histidine-rich domain and a CPD. Similarly, these polypeptides can be used for the delivery of CRISPR-effector based RNPs in eukaryotic cells.

In example embodiments, programmable nucleases are directed to specific genomic loci using guide molecules encoded for by the vectors for use in cryptography. In preferred embodiments, in order to hide the base edits within the genome or to hide that a cell has been encrypted with information, the vector(s) used to introduce guide molecules into cells is not integrated or replicated during cell division. Thus, in example embodiments, plasmids, non-integrating viral vectors, or RNPs are delivered to host cells. In example embodiments, guide molecule vectors are delivered by liposomes, nanoparticles, exosomes, microvesicles, electroporation, or a gene-gun. In one example, transient transfection is used to introduce the CRISPR components into the cell, such that no DNA encoding a guide RNA or Cas9 are incorporated into the cell's genome. In another example, an adenovirus is used because the adenovirus does not integrate into the host genome and expression of transgenes is transient (see, e.g., Chu D, Sullivan CC, Weitzman M D, et al. Direct comparison of efficiency and stability of gene transfer into the mammalian heart using adeno-associated virus versus adenovirus vectors. J Thorac Cardiovasc Surg. 2003; 126(3):671-679). In another example, an AAV is used because it can be used for transient expression.

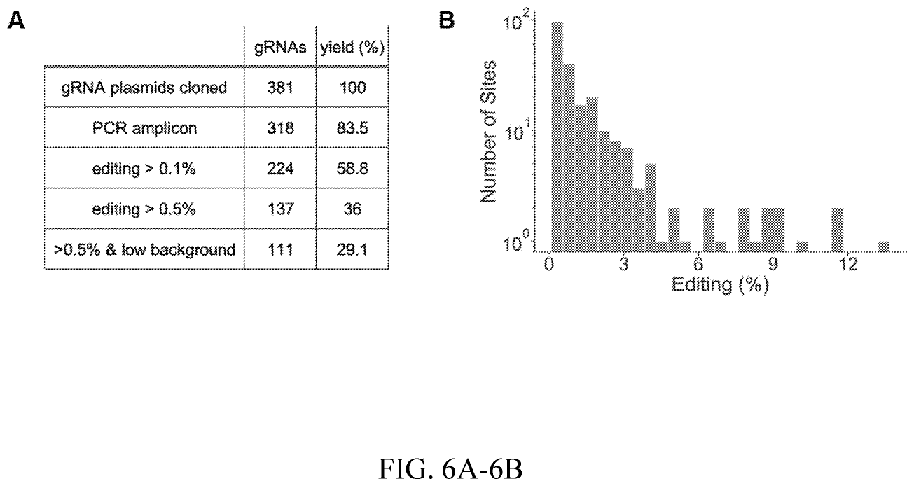

In example embodiments, the one or more guide molecules and/or one or more programmable nucleases described herein are encoded in mRNA. In example embodiments, mRNA may be delivered as naked RNA (with or without modification). In example embodiments, the mRNA may further comprise a delivery platform. Example delivery planforms include, but are not limited to liposomes, conjugates, peptides, exosomes, polymers, dendrimers, and inorganic nanoparticles. Example liposomes include Dlin-DMA, Dlin-MC3-DMA, and EnCore. Example conjugates include GalNAc, cholesterol, and RGD. Example polymers include cyclodextrin, PBAVE, PEI, and PLGA. Example peptides include DPC2.0 (MLP), and PNP. Example delivery platforms are described in Hu et al. “Therapeutic siRNA: state of the art” Signal Transduction and Targeted Therapy 5, Article number 100 (2020), particularly pages 11-20 and FIG. 6, which are incorporated herein by reference.

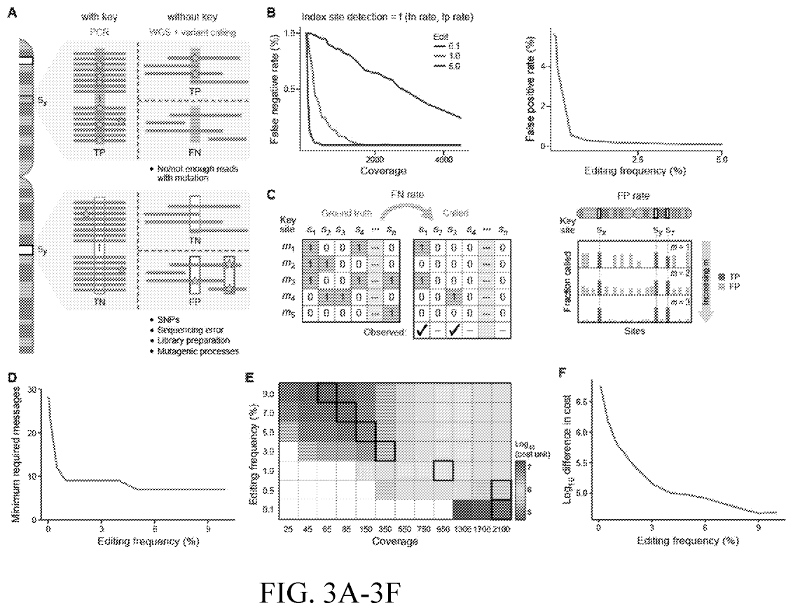

The mRNA modalities described above may comprise one or more modifications including, but not limited to, base modification, ribose modifications, and phosphate modifications. Example base modifications may include 2′-O-methyl, 2′0-methoxyethyl, 2′-arabinoo-fluoro, 2′-O-benzyl, 2′-O-methyl-4-pyridine, locked nucleic acid (LNA), (S)-cEt-BNA, tricyclo-DNA, PMO, unlocked nucleic acid, and glycol nucleic acid. Phosphate modifications include phophoorothioate (PS, Rp isomer, and PS, Sp isomer), phosphorodithioate, methylphosphonate, methoxypropyl-phosphonate, 5′-(E)-vinylphosphonate, 5′-Methyl Phosphonate, (S)-5′-C-methyl with phosphate, 5′-phosphorothioate,and peptide nucleic acid. Base modifications may include pseudouridine, 2′-thiouridine, N6′-methyladenosine, 5′-methylcytidine, 5′-fluoro-2′-deoxyuridine, N-ethylpiperidine 7′-EAA triazole modified adenine, N-ethylpiperidine 6′-triazole modified adenine, 6′-phenylpyrrolo-cytosinie, 2′,4′-difluorotoluly ribonucleoside, and 5′-nitroindole. A summary of modifications and example locations within a RNAi polynucleotide for each modification are described in Hu et al. “Therapeutic siRNA: state of the art” Signal Transduction and Targeted Therapy 5, Article number 100 (2020), particularly FIGs 2 and 3, which are incorporated herein by reference.

### Screening For Editing Efficiency

A further aspect relates to a method for screening one or more enrichment markers of a cell or cell population as disclosed herein, comprising: a) applying a set of guide molecules and one or more programmable nucleases to the cell or cell population; and b) detecting modulation of one or more enrichment markers of the cell or cell population by the set of guide molecules and one or more programmable nucleases. The enrichment markers of the cell or cell population that is modulated may be editing efficiency, an exogenous reporter, or a combination thereof. The editing efficiency may be a measure of a gene signature or biological program specific to a cell type or cell phenotype or phenotype specific to a population of cells (e.g., an inflammatory phenotype or suppressive immune phenotype). The editing efficiency may indicate the amount (either as a number, ratio, or percentage) of genomic loci edited in the population of cells. For example, the editing frequency of genomic locus X could be 1 out of 1000 genomic loci X in the plurality of genomic loci, or the editing frequency could be 1/1000^{th}, or the editing frequenting could be 0.1%. In certain embodiments, steps can include administering candidate programmable nucleases to cells, detecting identified cell (sub)populations for changes in signatures, or identifying relative changes in cell (sub) populations which may comprise detecting relative abundance of particular gene signatures.

In example embodiments, editing efficiency is determined by sequencing one or more genomic loci having a low overall editing efficiency in the plurality of genomic loci, wherein an edit above an editing efficiency threshold at the one or more genomic loci is an indicator that a cell comprises all edits in the set of edits. A genomic locus having a low overall editing efficiency refers to a genomic locus having an editing efficiency at least less than a genomic locus with the highest editing efficiency among the plurality of genomic loci. According, one or more low editing efficiencies may be used to for the enrichment method described herein. For example, the lowest editing efficiency may be used, the second lowest editing efficiency may be used, the third lowest editing efficiency may be used, etc., or a combination of low editing efficiencies may be used. In example embodiments, the editing efficiency threshold is at least 0.1%, 0.2%, 0.5%, or 1%. In example embodiments, the editing efficiency threshold is at maximum 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, 3.0%, 3.1%, 3.2%, 3.3%, 3.4%, 3.5%, 3.6%, 3.7%, 3.8%, 3.9%, 4.0%, 4.1%, 4.2%, 4.3%, 4.4%, 4.5%, 4.6%, 4.7%, 4.8%, 4.9% 5.0%, 5.1%, 5.2%, 5.3%, 5.4%, 5.5%, 5.6%, 5.7%, 5.8%, 5.9%, or 6.0%.

In example embodiments, the one or more genomic loci having a low overall editing efficiency are associated with a functional phenotype or an encoded reporter gene, wherein the functional phenotype or encoded reported gene are expressed if the one or more genomic loci having the lowest overall editing efficiency are successfully edited. In example embodiments, the encoded reporter gene may include beta-galactosidase, beta-lactamase, alkaline phosphatase and an optically active protein (e.g., a fluorescent or luminescent protein, including but not limited to green fluorescence protein GFP and luciferase. In example embodiments, the functional phenotype is antibiotic resistance.

In example embodiments, the exogenous reporter is co-delivered to the population of cells. In some methods, the exogenous reporter is a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996). In example embodiments, the exogenous reporter is a fluorescent protein.

In example embodiments, one or more cells that express the exogenous reporter above an expression threshold are isolated. In example embodiments, the expression threshold is in a top 20%, 15%, 10%, 5%, or 1% of the edited population of cells. In example embodiments, the expression threshold is in a top 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50%. In example embodiments, luminescence, absorbance and fluorescence detection methods may be used measure the encoded reporter gene or exogenous reporter. For example, the expression threshold may include cells whose measured luminescence, absorbance, or fluorescence is in a designated percentage of the edited population of cells.

The term “edit” broadly denotes a qualitative and/or quantitative alteration, change or variation in that which is being modulated. Where editing can be assessed quantitatively—for example, where editing comprises or consists of a change in a quantifiable variable such as a quantifiable property of a cell or where a quantifiable variable provides a suitable surrogate for the editing—editing specifically encompasses both increase (e.g., activation) or decrease (e.g., inhibition) in the measured variable. The term encompasses any extent of such editing, e.g., any extent of such increase or decrease, and may more particularly refer to statistically significant increase or decrease in the measured variable. Preferably, editing may be specific or selective, hence, one or more desired phenotypic aspects of an immune cell or immune cell population may be modulated without substantially altering other (unintended, undesired) phenotypic aspect(s).

### Sequencing Methods

In example embodiments, an edited single cell/nuclei cDNA library is utilized to obtain consensus sequences for a variable repeat region and transcriptome data from single cells. As used herein the term “transcriptome” refers to the set of transcript molecules. In some embodiments, transcript refers to RNA molecules, e.g., messenger RNA (mRNA) molecules, small interfering RNA (siRNA) molecules, transfer RNA (tRNA) molecules, ribosomal RNA (rRNA) molecules, and complimentary sequences, e.g., cDNA molecules. In some embodiments, a transcriptome refers to a set of mRNA molecules. In some embodiments, a transcriptome refers to a set of cDNA molecules. In some embodiments, a transcriptome refers to one or more of mRNA molecules, siRNA molecules, tRNA molecules, rRNA molecules, in a sample, for example, a single cell or a population of cells. In some embodiments, a transcriptome refers to cDNA generated from one or more of mRNA molecules, siRNA molecules, tRNA molecules, rRNA molecules, in a sample, for example, a single cell or a population of cells. In some embodiments, a transcriptome refers to 50%, 55, 60, 65, 70, 75, 80, 85, 90, 91, 92, 93, 94, 95, 96, 97, 98, 99, 99.9, or 100% of transcripts from a single cell or a population of cells. In some embodiments, transcriptome not only refers to the species of transcripts, such as mRNA species, but also the amount of each species in the sample. In some embodiments, a transcriptome includes each mRNA molecule in the sample, such as all the mRNA molecules in a single cell. The barcoded single cell/nuclei cDNA library can be prepared by any method known in the art.

Example embodiments involve single cell RNA sequencing (see, e.g., Qi Z, Barrett T, Parikh AS, Tirosh I, Puram SV. Single-cell sequencing and its applications in head and neck cancer. Oral Oncol. 2019; 99:104441; Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p666-673, 2012).

Example embodiments involve plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

Example embodiments involve high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO 2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO 2016/168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO 2014/210353A2; Zilionis, et al., 2017, “Single-cell barcoding and sequencing using droplet microfluidics” Nat Protoc. Jan; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273, all the contents and disclosure of each of which are herein incorporated by reference in their entirety.

Example embodiments involve single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; International Patent Application No. PCT/US2016/059239, published as WO2017164936 on Sep. 28, 2017; International Patent Application No. PCT/US2018/060860, published as WO/2019/094984 on May 16, 2019; International Patent Application No. PCT/US2019/055894, published as WO/2020/077236 on Apr. 16, 2020; Drokhlyansky, et al., “The enteric nervous system of the human and mouse colon at a single-cell resolution,” bioRxiv 746743; doi: doi.org/10.1101/746743; and Drokhlyansky E, Smillie CS, Van Wittenberghe N, et al. The Human and Mouse Enteric Nervous System at Single-Cell Resolution. Cell. 2020; 182(6):1606-1622.e23, which are herein incorporated by reference in their entirety.

In example embodiments, generating a single cell/nuclei cDNA library includes WTA and the term “cDNA library” can be used interchangeably with “WTA library.” In example embodiments, reverse transcription (RT) and whole transcriptome amplification (WTA) results in a library of complementary DNA (cDNA) molecules tagged with a cell barcode and UMI. In example embodiments, whole transcriptome amplification (WTA) is used to generate the cDNA library. The cDNA library may also be referred to as the whole transcriptome amplification (WTA) library. The library may include “WTA products”. “Whole transcriptome amplification” (“WTA”) refers to any amplification method that aims to produce an amplification product that is representative of a population of RNA from the cell from which it was prepared. An illustrative WTA method entails production of cDNA bearing linkers on either end that facilitate unbiased amplification. In many implementations, WTA is carried out to analyze messenger (poly-A) RNA (this is also referred to as “RNAseq”). WTA may include reverse transcription (RT) to generate first strand cDNA. First strand synthesis may be followed by second strand synthesis. First strand synthesis may include priming of the RT on a 3′ adaptor linked to the RNA molecules. In example embodiments, each RNA in a library may be amplified to create a whole transcriptome amplified (WTA) RNA by reverse transcription with a primer comprising a sequence adapter. The reverse transcribed product may be amplified by PCR amplification with primers that bind both 5′ and 3′ sequence adapters. In example embodiments, the amplified RNA comprises the orientation: 5′-sequencing adapter—cell barcode—unique molecular identifier (UMI) -UUUUUUU—mRNA-3′. In some embodiments, PCR amplification is conducted on the reverse transcribed products with primers that bind both sequence adapters and add a library barcode and optionally additional sequence adapters.

In example embodiments, any suitable RNA or DNA amplification technique may be used. In example embodiments, the RNA or DNA amplification is an isothermal amplification. In example embodiments, the isothermal amplification may be nucleic-acid sequenced-based amplification (NASBA), recombinase polymerase amplification (RPA), loop-mediated isothermal amplification (LAMP), strand displacement amplification (SDA), helicase-dependent amplification (HDA), or nicking enzyme amplification reaction (NEAR). In example embodiments, non-isothermal amplification methods may be used which include, but are not limited to, PCR, multiple displacement amplification (MDA), rolling circle amplification (RCA), ligase chain reaction (LCR), or ramification amplification method (RAM).

In example embodiments, transcripts comprising a variable repeat region are enriched using primers that are specifically designed to amplify cDNAs comprising the variable repeat region. In example embodiments, the WTA product (cDNA library) can be used as starting material to enrich full length cDNAs comprising the variable repeat region. In example embodiments, the cDNAs can be enriched from a single cell library by amplification with primers specific to the target cDNA (e.g., a combination of primers specific to the cDNA and an adapter sequence). In exemplary embodiments, a PCR primer pair includes a constant primer specific to one end of a paired end sequencing library (e.g., forward or reverse primer) and a second primer (e.g., forward or reverse primer) that is complementary to a gene specific sequence. In some embodiments, amplification can be performed in a multiplexed manner, wherein multiple target nucleic acid sequences are amplified simultaneously. In example embodiments, amplification is performed with one or more spike-in primers that target the gene transcript encompassing the variable repeat region 5′ of the variable repeat region.

In some embodiments, amplification can be performed using a polymerase chain reaction (PCR). As used herein, PCR can refer to a reaction for the in vitro amplification of specific DNA sequences by the simultaneous primer extension of complementary strands of DNA. As used herein, PCR can encompass derivative forms of the reaction, including but not limited to, RT-PCR, real-time PCR, nested PCR, quantitative PCR, multiplexed PCR, digital PCR, and assembly PCR. Amplification can be performed in one or more rounds. In some instances, there are multiple rounds of amplification. Amplification can comprise two or more rounds of amplification.

Another targeted PCR based sequencing method, developed by Raindance (Billerica, MA) uses microdroplet PCR and custom-designed droplet libraries (Tewhey, et al. Microdroplet-based PCR enrichment for large-scale targeted sequencing. Nat Biotechnol. 2009; 27:1025-1031). The nature of micro-droplet emulsion PCR significantly decreases PCR amplification bias (Hori et al., Uniform amplification of multiple DNAs by emulsion PCR. Biochem Biophys Res Commun. 2007; 352:323-328). Microdroplet PCR allows the user to set up 1.5×10⁶ micro-droplet amplifications in a single tube in under an hour. The droplet libraries are designed based on 500 bp amplicons, and a single custom library can target from 2000 to 10,000 different amplicons covering up to 5×10⁶ bases. In example embodiments, barcoded cDNAs are enriched by Microdroplet-based PCR enrichment. Micro-droplet emulsion PCR may advantageously be used to eliminate amplification bias caused by primer interactions in the case where multiple cDNA sequences are enriched. The discrete encapsulation of microdroplet PCR reactions prevents possible primer pair interactions allowing for highly efficient simultaneous amplification of up to 4,000 targeted sequences and greatly reduces the amount of reagents required.

In example embodiments, amplifying target sequences may include other PCR methods (e.g., Cold-PCR).

In example embodiments, amplification products are labeled with biotin. Biotin labeled amplicons may be isolated using an affinity agent (e.g., streptavidin beads), such that only amplified products are recovered.

In example embodiments, the enriched amplification products are size selected to separate longer amplification products from shorter amplification products. In example embodiments, the amplification products are separated by size into 2, 3, 4, or 5 groups, preferably 2. A classic method for isolating DNA of a target size involves the separation of the DNA in a gel, cutting out the desired gel band(s) and then isolating the DNA of the target size from the gel fragment(s). Another widely used technology is the size selective precipitation with polyethylene glycol based buffers (Lis and Schleif Nucleic Acids Res. 1975 March; 2(3):383-9) or the binding/precipitation on carboxyl-functionalized beads (DeAngelis et al, Nuc. Acid. Res. 1995, Vol 23(22), 4742-3; U.S. Pat. Nos. 5,898,071 and 5,705,628, commercialized by Beckman-Coulter (AmPure XP; SPRIselect) and U.S. Pat. No. 6,534,262). In another method, DNA is bound to a silicon containing surface of a binding matrix in the presence of a chaotropic salt and the size of DNA molecules that bind to the binding matrix can be controlled by the pH value of the binding mixture (see, e.g., U.S. Pat. No. 10,745,686 B2).

In example embodiments, the WTA product (cDNA library) can be used as starting material to enrich cDNAs comprising the variable repeat region and used as starting material for single cell/nuclei RNA-seq library generation. The libraries generated can then be sequenced by long read sequencing and short read sequencing, respectively.

In example embodiments, sequencing comprises high-throughput (formerly “next-generation”) technologies to generate sequencing reads. In DNA sequencing, a read is an inferred sequence of base pairs (or base pair probabilities) corresponding to all or part of a single DNA fragment. A typical sequencing experiment involves fragmentation of the genome into millions of molecules or generating complementary DNA (cDNA) fragments, which are size-selected and ligated to adapters. The set of fragments is referred to as a sequencing library, which is sequenced to produce a set of reads. Methods for constructing sequencing libraries are known in the art (see, e.g., Head et al., Library construction for next-generation sequencing: Overviews and challenges. Biotechniques. 2014; 56(2): 61-77; and Trombetta, J. J., Gennert, D., Lu, D., Satija, R., Shalek, A. K. & Regev, A. Preparation of Single-Cell RNA-Seq Libraries for Next Generation Sequencing. Curr Protoc Mol Biol. 107, 4 22 21-24 22 17, doi:10.1002/0471142727.mb0422s107 (2014). PMCID:4338574). A “library” or “fragment library” may be a collection of nucleic acid molecules derived from one or more nucleic acid samples, in which fragments of nucleic acid have been modified, generally by incorporating terminal adapter sequences comprising one or more primer binding sites and identifiable sequence tags. In example embodiments, the library members (e.g., genomic DNA, cDNA) may include sequencing adaptors that are compatible with use in, e.g., Illumina's reversible terminator method, long read nanopore sequencing, Roche's pyrosequencing method (454), Life Technologies' sequencing by ligation (the SOLiD platform), PacBio's long read sequencing, or Life Technologies' Ion Torrent platform. Recent advances in long-read sequencing have enabled sequencing full-length transcripts; Pacific Biosciences (PacBio) single-molecule real-time (SMRT) sequencing and Oxford Nanopore Technologies (ONT) nanopore sequencing can generate reads >10 Kb (see, e.g., Amarasinghe SL, Su S, Dong X, Zappia L, Ritchie ME, Gouil Q. Opportunities and challenges in long-read sequencing data analysis. Genome Biol. 2020; 21(1):30). Examples of such methods are described in the following references: Margulies et al (Nature 2005 437: 376-80); Schneider and Dekker (Nat Biotechnol. 2012 Apr. 10; 30(4):326-8); Ronaghi et al (Analytical Biochemistry 1996 242: 84-9); Shendure et al (Science 2005 309: 1728-32); Imelfort et al (Brief Bioinform. 2009 10:609-18); Fox et al (Methods Mol. Biol. 2009; 553:79-108); Appleby et al (Methods Mol. Biol. 2009; 513:19-39); Wenger, A. M., et al. (2019) Accurate circular consensus long-read sequencing improves variant detection and assembly of a human genome. Nature Biotechnology, 37, 1155-1162; Leung SK, Jeffries AR, Castanho I, et al. Full-length transcript sequencing of human and mouse cerebral cortex identifies widespread isoform diversity and alternative splicing. Cell Rep. 2021; 37(7):110022; Gordon SP, Tseng E, Salamov A, et al. Widespread Polycistronic Transcripts in Fungi Revealed by Single-Molecule mRNA Sequencing. PLoS One. 2015; 10(7):e0132628; and Morozova et al (Genomics. 2008 92:255-64), which are incorporated by reference for the general descriptions of the methods and the particular steps of the methods, including all starting products, reagents, and final products for each of the steps.

### Perturb-Seq

In some embodiments, the systems and methods for multi-site editing described here can be used to introduce perturbations for use in a Perturb-Seq screening. In certain embodiments, gene signatures are screened by perturbation of target genes within said signatures. In some embodiments, perturbation of the target genes is done using the systems and methods for multi-site editing of the present disclosure. Methods and tools for genome-scale screening of perturbations in single cells using programmable nucleases have been described, herein referred to as perturb-seq and can be adapted for use with the systems and methods described herein (see e.g., Dixit et al., “Perturb-Seq: Dissecting Molecular Circuits with Scalable Single-Cell RNA Profiling of Pooled Genetic Screens” 2016, Cell 167, 1853-1866; Adamson et al., “A Multiplexed Single-Cell CRISPR Screening Platform Enables Systematic Dissection of the Unfolded Protein Response” 2016, Cell 167, 1867-1882; Jaitin DA, Weiner A, Yofe I, et al. Dissecting Immune Circuits by Linking CRISPR-Pooled Screens with Single-Cell RNA-Seq. Cell. 2016; 167(7):1883-1896.e15; Feldman et al., Lentiviral co-packaging mitigates the effects of intermolecular recombination and multiple integrations in pooled genetic screens, bioRxiv 262121, doi: doi.org/10.1101/262121; Datlinger, et al., 2017, Pooled CRISPR screening with single-cell transcriptome readout. Nature Methods. Vol. 14 No. 3 DOI: 10.1038/nmeth.4177; Hill et al., On the design of CRISPR-based single cell molecular screens, Nat Methods. 2018 April; 15(4): 271-274; Replogle, et al., “Combinatorial single-cell CRISPR screens by direct guide RNA capture and targeted sequencing” Nat Biotechnol (2020). doi.org/10.1038/s41587-020-0470-y; Schraivogel D, Gschwind AR, Milbank J H, et al. “Targeted Perturb-seq enables genome-scale genetic screens in single cells”. Nat Methods. 2020; 17(6):629-635; Frangieh CJ, Melms JC, Thakore P I, et al. Multimodal pooled Perturb-CITE-seq screens in patient models define mechanisms of cancer immune evasion. Nat Genet. 2021; 53(3):332-341; US patent application publication number US 2020/0283843A1; and U.S. Pat. No. 11,214,797 B2). The present disclosure is compatible with perturb-seq, such that signature genes may be perturbed and the perturbation may be identified and assigned to the proteomic and gene expression readouts of single cells. In certain embodiments, signature genes may be perturbed in single cells and gene expression analyzed. Not being bound by a theory, networks of genes that are disrupted due to perturbation of a signature gene may be determined. Understanding the network of genes effected by a perturbation may allow for a gene to be linked to a specific pathway that may be targeted to modulate the signature and treat a cancer. Thus, in certain embodiments, perturb-seq is used to discover novel drug targets to allow treatment of specific cancer patients having the gene signature as disclosed herein.

The perturbation methods and tools allow reconstructing of a cellular network or circuit. In one embodiment, the method comprises (1) introducing single-order or combinatorial perturbations to a population of cells, (2) measuring genomic, genetic, proteomic, epigenetic and/or phenotypic differences in single cells and (3) assigning a perturbation(s) to the single cells. Not being bound by a theory, a perturbation may be linked to a phenotypic change, preferably changes in gene or protein expression. In preferred embodiments, measured differences that are relevant to the perturbations are determined by applying a model accounting for co-variates to the measured differences. The model may include the capture rate of measured signals, whether the perturbation actually perturbed the cell (phenotypic impact), the presence of subpopulations of either different cells or cell states, and/or analysis of matched cells without any perturbation. In certain embodiments, the measuring of phenotypic differences and assigning a perturbation to a single cell is determined by performing single cell RNA sequencing (RNA-seq). In preferred embodiments, the single cell RNA-seq is performed by any method as described herein (e.g., Drop-seq, InDrop, 10× genomics). In certain embodiments, a guide RNA is detected by RNA-seq using a transcript expressed from a vector encoding the guide RNA.

In certain embodiments, whole genome screens can be used for understanding the phenotypic readout of perturbing potential target genes. In preferred embodiments, perturbations target expressed genes as defined by a gene signature using a focused guide molecule library. Libraries may be focused on expressed genes in specific networks or pathways. In other preferred embodiments, regulatory drivers are perturbed. In certain embodiments, Applicants perform systematic perturbation of key genes that regulate T-cell function in a high-throughput fashion. In certain embodiments, Applicants perform systematic perturbation ofkey genes that regulate cancer cell function in a high-throughput fashion (e.g., immune resistance or immunotherapy resistance). Applicants can use gene expression profiling data to define the target of interest and perform follow-up single-cell and population RNA-seq analysis. Not being bound by a theory, this approach will accelerate the development of therapeutics for human disorders, in particular cancer. Not being bound by a theory, this approach will enhance the understanding of the biology of T-cells and tumor immunity, and accelerate the development of therapeutics for human disorders, in particular cancer, as described herein.

Not being bound by a theory, perturbation studies targeting the genes and gene signatures described herein could (1) generate new insights regarding regulation and interaction of molecules within the system that contribute to suppression of an immune response, such as in the case within the tumor microenvironment, and (2) establish potential therapeutic targets or pathways that could be translated into clinical application.

In certain embodiments, after determining perturbation effects in cancer cells and/or primary T-cells, the cells are infused back to the tumor xenograft models (melanoma, such as B16F10 and colon cancer, such as CT26) to observe the phenotypic effects of genome editing. Not being bound by a theory, detailed characterization can be performed based on (1) the phenotypes related to tumor progression, tumor growth, immune response, etc. (2) the TILs that have been genetically perturbed by CRISPR-Cas9 can be isolated from tumor samples, subject to cytokine profiling, qPCR/RNA-seq, and single-cell analysis to understand the biological effects of perturbing the key driver genes within the tumor-immune cell contexts. Not being bound by a theory, this will lead to validation of TILs biology as well as lead to therapeutic targets.

In example embodiments, screening is conducted in replicate and selecting the one or more cells comprises selecting one or more cells with a correlated enrichment. For example, the screening may be performed iteratively. A correlated enrichment may include selecting one or more cells with the same or similar amount (e.g., number, ratio, percentage) of enrichment markers. A similar amount of enrichment markers may include a difference of at minimum 1%, 2%, 3%, 4%, 5%, 6%, 7%, 8%, 9%, or 10% between correlated cells.

### Isolation of Cells

In example embodiments, isolating one or more cells from the population of cells that comprise the one or more enrichment markers. The isolation of one or more cells may depend on: level of automation; ability to isolate specific/individual cells; and compatibility with certain application requirements. Isolation methods include, but are not limited to, FACS/flow cytometry, manual cell picking, laser microdissection, random seeding/limiting dilution, microfluidics/lab-on-a-chip devices, electric fields (e.g., dielectrophoresis), capillary isolation, non-contact dispensing/printing, and optical tweezers. See e.g., Gross, A., Schoendube, J., Zimmermann, S., Steeb, M., Zengerle, R. and Koltay, P., 2015. Technologies for single-cell isolation. International journal of molecular sciences, 16(8), pp.16897-16919.

### Method of Introducing Multi-Site Edits in Cells

In one aspect, as described herein, are methods of introducing multi-site edits in cells and subsequent enrichment. The methods include, but are not limited to, cell factories, authentication of biological material, cell-based therapies, xenotransplantation, agricultural products, and applications in non-human animals.

### Types of Edited Cells/Applications

In one aspect, embodiments disclosed herein are directed to biological materials that have been modified as disclosed herein. In example embodiments, a biological material is a modified organism or a modified cell. In example embodiments, the modified cells are from a prokaryote, a eukaryote, or a combination thereof. In example embodiments, the modified cell(s) is a primary cell(s). A primary cell generally refers to a cell isolated or harvested from tissue or an organ. In some cases, a primary cell is terminally differentiated cells. The terminally differentiated cells may be a specific type and include a high level of function. For example, the modified cells can include any cell line or primary cell, such as HEK293T cells. The cell(s) may comprise a cell from or in a model non-human organism. In example embodiments, the modified cell(s) is a stem cell(s). Stem cells may include undifferentiated or partially differentiated cells, wherein these cells can differentiate into different types of cells. Stem cells can proliferate indefinitely to produce more of the same stem cell. The stem cell may be an adult or embryonic stem cell. For example, the modified cells may include any cell line or stem cell, such as embryonic stem cells (ESCs).

In one aspect, an engineered, non-naturally occurring cell, or progeny thereof, wherein the genome of the cell is modified according to the methods and systems described herein. The modified cells may be generated using the gene editing systems described herein. In example embodiments, the modified cell is a therapeutic cell. Clinical application of CRISPR-Cas9 gene-edited T cells is generally safe and feasible (see, e.g., Lu Y, Xue J, Deng T, et al. Safety and feasibility of CRISPR-edited T cells in patients with refractory non-small-cell lung cancer [published correction appears in Nat Med. 2020 July; 26(7):1149]. Nat Med. 2020; 26(5):732-740; Lacey SF, Fraietta JA. First Trial of CRISPR-Edited T cells in Lung Cancer. Trends Mol Med. 2020; 26(8):713-715; and Zhang X, Cheng C, Sun W, Wang H. Engineering T Cells Using CRISPR/Cas9 for Cancer Therapy. Methods Mol Biol. 2020; 2115:419-433). Immune cells can also be edited ex vivo using Zn Finger proteins (see, e.g., Perez EE, Wang J, Miller J C, et al. Establishment of HIV-1 resistance in CD4+ T cells by genome editing using zinc-finger nucleases. Nat Biotechnol. 2008; 26(7):808-816).

### Cell Factories

In example embodiments, as described herein, is a method of enriching for multi-site edited cell factories. In cell factory engineering, one or more cells can be edited to make one or more compounds of interest. Cell factory engineering can be organized into three (3) categories: 1) optimization of the production of a metabolite in the native host; 2) production of a non-native metabolite by expression of a heterologous biosynthetic pathway; and 3) expression of a heterologous protein.

Cell factories engineered for production of native metabolites are modified to produce naturally, either intracellularly or secreted, a naturally occurring compound. For example, amino and nucleic acids, antibiotics, vitamins, enzymes, bioactive compounds, and proteins produced from anabolic pathways of cells. Cell factories may be engineered for the production of native metabolites by: pathway overexpression; transporter engineering; de-branching; product degradation; co-factor engineering; removal of feedback inhibition; by-product elimination; precursor/substrate enrichment; de-regulation of carbon catabolism; or signal transduction.

Cell factories may, for example, be engineered for production of a non-native metabolite by expression of a heterologous biosynthetic pathway. For example, the cell factory is engineered based on the choice of production in the native host or transfer of the pathway to another well-known host. Considerations for cell factories engineered for the production of a non-native metabolite by expression of a heterologous biosynthetic pathway include: compartmentalization or steric proximity; co-factor availability; substrate and co-substrate availability; product efflux pumps; biosynthesis of functional groups; and transcription engineering.

Cell factories may, for example, be engineered for the expression of a heterologous protein. A cell factory of for the expression of a heterologous protein is chosen based on the properties and applications of the desired protein. Considerations for cell factories engineered for the expression of a heterologous protein include: promoter engineering; gene fusion for enhanced secretion; stability of heterologous gene transcripts; improved translocation; proteins secretion stress engineering; engineering the post-translational modification machinery; improved vesicle trafficking; protein glycosylation; protease deletions; and by-product removal. See e.g., Davy, A. M.; Kildegaard, H. F.; Andersen, M. R. Cell Factory Engineering. Cell Systems, 2017, 4, 262-275.

### Authentication of Biological Material

In example embodiments, as described herein, a method of enriching for multi-site edited cells that encode an authentication signature into a biological material. Encoding may comprise an encrypted verification signature in one or more genomes of the biological material by introducing edits using one or more programable nuclease at a plurality of genomic loci defined according to an encryption key, whereby measuring the plurality of the genomic loci as defined by the encryption key can be used to identify and/or authenticate the origin or source of the biological material. A verification signature may be any type of information, such as those described herein, associated with the biological material. Information associated with the biological material may be a biological material identification code such as a numerical ID, alphabet ID, or combination thereof.

In one aspect, as described herein, a method of authenticating a biological material, comprising adding one or more cells to the biological material, the one or more cells comprising information encrypted in a genome(s) of the one or more cells, wherein the encrypted information is used to authenticate the biological material.

In one aspect, as described herein, a method of enriching for multi-site edited cells comprising authenticated biological material comprising: measuring a set of genomic loci from one or more cells obtained from the biological material and as defined by an encryption key, wherein at least a portion of the cells of the biological material comprises genomes previously edited with one or more nucleic acid modifying agents to encode an authentication code according to the encryption key; wherein an observed allele status at the genomic loci, in combination with the encryption key, are used to decode the authentications signature that confirms an identity of and/or authenticates an origin of the biological material.

Biological authentication is a necessary precaution to prevent cross-contamination, misidentification (e.g., species determination), tampering, or misuse of biological material. The NIH requires authentication of biological material to receive funding grants and the FDA requires authentication of biological material included in investigational new drug applications. Current approaches rely on comparing the genomes of biological material to reference-quality whole genome sequences. The compositions, systems, and methods herein rely on a plurality of genomic loci according to an encryption key to authenticate biological material. Consequently, only the portion of the genome according to the encryption key needs to be sequenced to authenticate the biological material.

In example embodiments, a reproduced biological material comprising an authentication signature or other encrypted information can be authenticated by measuring the allele frequency of the authentication signature or other encrypted information. If the allele frequency is identical to the original biological material, then the reproduced biological material is validated. If the allele frequency is different, then the reproduced biological material has been altered from that of the original biological material.

In example embodiments, compositions, systems, and methods herein can be used for genome encryption in multi-cultures, which includes co-cultures. Multi-cultures attempt to replicate systems of tissues or ecologies to model complex interactions. Genomic encryption can be used to authenticate the process of multi-cultures or track changes to the systems overtime. See e.g., Goers, L.; Freemont, P.; Polizzi, K. M. Co-Culture Systems and Technologies: Taking Synthetic Biology to the next Level. Journal of The Royal Society Interface, 2014, 11, 20140065 and Diender, M.; Parera Olm, I.; Sousa, D. Z. Synthetic Co-Cultures: Novel Avenues for Bio-Based Processes. Current Opinion in Biotechnology, 2021, 67, 72-79.

In example embodiments, compositions, systems, and methods herein can be used for genome encryption in cell-based sensors, including cell-based screens. Cell-based sensors are used, for example, to detect changes in the environment (e.g., sample toxicity or soil conditions) or pharmacology (e.g., drug screening). Cell-based sensors use transduction/detection methods such as electrical cell-substrate impedance sensing (ECIS), light addressable potentiometric sensor, and fluorescent imaging. The engineered cells for cell-based sensing may comprise authentication signatures or otherwise encrypted information. See e.g., Gheorghiu, M. A Short Review on Cell-Based Biosensing: Challenges and Breakthroughs in Biomedical Analysis. The Journal of Biomedical Research, 2021, 35, 255.

In example embodiments, compositions, systems, and methods herein can be used for genome encryption and/or authentication in cell-based models, such as disease and drug models including Organ-on-a-Chip. See e.g., Ma, C.; et al. Organ-on-a-Chip: A New Paradigm for Drug Development. Trends in Pharmacological Sciences, 2021, 42, 119-133, Wu, Q.; et al. Organ-on-a-Chip: Recent Breakthroughs and Future Prospects. BioMedical Engineering OnLine, 2020, 19.

### Cell-Based Therapies

In example embodiments, wherein the biological material is a modified organism or a modified cell, such as those described elsewhere herein, the modified cell may comprise a therapeutic cell. In some embodiments, the therapeutic cells can be used in cell-based therapies. A method of a cell therapy generally includes administering, using a suitable method or technique, a modified cell or cell population (or a pharmaceutical formulation thereof) to a subject in need thereof. It will be appreciated that the cells can be autologous or allogeneic. In some embodiments, the modified cells are allogeneic and include modifications so as to reduce the recipient's immune or other response to the modified cells to increase efficacy of the therapy. In some embodiments, where the modified cell(s) are allogeneic, the method can comprise administering the cells with one or more protective biomaterials that are capable of shielding the allogenic cells from the recipient's immune system.

Cell-based therapies may include regenerative and tissue and/or organ replacement therapies. For example, replacement therapies may include adoptive cell therapies (ACT). As used herein, “ACT”, “adoptive cell therapy” and “adoptive cell transfer” may be used interchangeably. In certain embodiments, Adoptive cell therapy (ACT) can refer to the transfer of cells to a patient with the goal of transferring the functionality and characteristics into the new host by engraftment of the cells (see, e.g., Mettananda et al., Editing an α-globin enhancer in primary human hematopoietic stem cells as a treatment for β-thalassemia, Nat Commun. 2017 Sep. 4; 8(1):424). As used herein, the term “engraft” or “engraftment” refers to the process of cell incorporation into a tissue of interest in vivo through contact with existing cells of the tissue. Adoptive cell therapy (ACT) can refer to the transfer of cells, most commonly immune-derived cells (e.g., T cells or NK cells), back into the same patient or into a new recipient host with the goal of transferring the immunologic functionality and characteristics into the new host. If possible, use of autologous cells helps the recipient by minimizing GVHTD issues. The adoptive transfer of autologous tumor infiltrating lymphocytes (TIL) (Zacharakis et al., (2018) Nat Med. 2018 June; 24(6):724-730; Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma, metastatic breast cancer and colorectal carcinoma, as well as patients with CD19-expressing hematologic malignancies (Kalos et al., (2011) Science Translational Medicine 3 (95): 95ra73). In certain embodiments, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

Cell-based replacement therapies may also comprise delivering keratinocytes, fibroblasts, bone marrow, and/or adipose tissue-derived mesenchymal stem cells to improve chronic wound healing by delivery of different cytokines, chemokines, and growth factors. See e.g., Domaszewska-Szostek, A.; et al. Cell-Based Therapies for Chronic Wounds Tested in Clinical Studies. Annals of Plastic Surgery, 2019, 83, e96-e109. Cell-based replacement therapies may also comprise replacement of beta, islet, CNS, neuron, tissue, or stem cell replacement therapies. See e.g., Brasile, L.; Stubenitsky, B. Will Cell Therapies Provide the Solution for the Shortage of Transplantable Organs?Current Opinion in Organ Transplantation, 2019, 24, 568-573, Yamanaka, S. Pluripotent Stem Cell-Based Cell Therapy—Promise and Challenges. Cell Stem Cell, 2020, 27, 523-531, and/or Madrid, M.; et al. Autologous Induced Pluripotent Stem Cell-Based Cell Therapies: Promise, Progress, and Challenges. Current Protocols, 2021, 1.

Cell-based regenerative and replacement therapies comprise engineering biological structures, such as tissue; organs; or a portion thereof, via in vitro fabrication. See e.g., Langer, R.; Vacanti, J. Advances in Tissue Engineering. Journal of Pediatric Surgery, 2016, 51, 8-12, Bakhshandeh, B.; et al. Tissue Engineering; Strategies, Tissues, and Biomaterials. Biotechnology and Genetic Engineering Reviews, 2017, 33, 144-172. Shafiee, A.; Atala, A. Tissue Engineering: Toward a New Era of Medicine. Annual Review of Medicine, 2017, 68, 29-40.

Cell-based therapies may also comprise administration of engineered cells for delivery of substances, e.g., drugs such as antibiotics, vaccines, and antibodies, for example where the cells are engineered via therapeutic bioreactors.

Cell-based therapies may also comprise administering engineered or otherwise modified microbiomes. Engineered microbiomes are used directly as treatment or preventing adverse effects from other therapies. For example, direct treatments using engineered microbiomes include fecal microbiota transplant, prebiotics, probiotics, synbiotics and synthetic microbes. Example preventative measures using microbes include drug reactivation (e.g., β-glucuronidases), drug deactivation (e.g., tyrosine decarboxylase), or toxic byproducts. See e.g., Khan, S.; Hauptman, R.; Kelly, L. Engineering the Microbiome to Prevent Adverse Events: Challenges and Opportunities. Annual Review of Pharmacology and Toxicology, 2021, 61, 159-179.

### Xenotransplantation

The present disclosure also contemplates use of the compositions, systems, and methods described herein for modified tissues for transplantation. Xenotransplantation comprises, for example, the use of RNA-guided DNA nucleases to knockout, knockdown or disrupt selected genes in an animal, such as a transgenic pig (such as the human heme oxygenase-1 transgenic pig line) or, for example, by disrupting expression of genes that encode epitopes recognized by the human immune system, i.e., xenoantigen genes. Candidate porcine genes for disruption may for example include α(1,3)-galactosyltransferase and cytidine monophosphate-N-acetylneuraminic acid hydroxylase genes (see International Patent Application Publication WO 2014/066505). In addition, genes encoding endogenous retroviruses may be disrupted, for example the genes encoding all porcine endogenous retroviruses (see Yang et al., 2015, Genome-wide inactivation of porcine endogenous retroviruses (PERVs), Science 27 Nov. 2015: Vol. 350 no. 6264 pp. 1101-1104). In addition, RNA-guided DNA nucleases may be used to target a site for integration of additional genes in xenotransplant donor animals, such as a human CD55 gene to improve protection against hyperacute rejection.

Xenotransplantation also relates to methods and compositions related to knocking out genes, amplifying genes and repairing particular mutations associated with DNA repeat instability and neurological disorders (Robert D. Wells, Tetsuo Ashizawa, Genetic Instabilities and Neurological Diseases, Second Edition, Academic Press, Oct. 13, 2011—Medical). Specific aspects of tandem repeat sequences have been found to be responsible for more than twenty human diseases (New insights into repeat instability: role of RNA·DNA hybrids. McIvor EI, Polak U, Napierala M. RNA Biol. 2010September-October; 7(5):551-8). Effector protein systems may be harnessed to correct these defects of genomic instability.

Xenotransplantation may also relate to correcting defects associated with a wide range of genetic diseases which are further described on the website of the National Institutes of Health under the topic subsection Genetic Disorders (website at health.nih.gov/topic/GeneticDisorders). The genetic brain diseases may include but are not limited to Adrenoleukodystrophy, Agenesis of the Corpus Callosum, Aicardi Syndrome, Alpers' Disease, Alzheimer's Disease, Barth Syndrome, Batten Disease, CADASIL, Cerebellar Degeneration, Fabry's Disease, Gerstmann-Straussler-Scheinker Disease, Huntington's Disease and other Triplet Repeat Disorders, Leigh's Disease, Lesch-Nyhan Syndrome, Menkes Disease, Mitochondrial Myopathies and NINDS Colpocephaly. These diseases are further described on the website of the National Institutes of Health under the subsection Genetic Brain Disorders.

### Agricultural Products

In example embodiments, the biological material is a modified organism or a modified cell, where the modified organism is a modified plant. In example embodiments, the method further comprises adding one or more cells to the biological material. The compositions, systems, and methods described herein can be used to perform gene or genome interrogation in plants and fungi. For example, the applications include investigation and/or selection and/or interrogations and/or comparison and/or manipulations and/or transformation of plant genes or genomes; e.g., to create, identify, develop, optimize, or confer trait(s) or characteristic(s) to plant(s) or to transform a plant or fungus genome. There can accordingly be improved production of plants, new plants with new combinations of traits or characteristics, or new plants with enhanced traits. The compositions, systems, and methods can be used with regard to plants in Site-Directed Integration (SDI) or Gene Editing (GE) or any Near Reverse Breeding (NRB) or Reverse Breeding (RB) techniques.

The compositions, systems, and methods herein may be used to authenticate/monitor desired traits (e.g., enhanced nutritional quality, increased resistance to diseases and resistance to biotic and abiotic stress, and increased production of commercially valuable plant products or heterologous compounds) on essentially any plants and fungi, and their cells and tissues. The compositions, systems, and methods may be used to authenticate/monitor endogenous genes or to authenticate/monitor their expression without the permanent introduction into the genome of any foreign gene.

In one embodiment, genome editing in plants or where RNAi or similar genome editing techniques have been used previously are used for genomic encryption; see, e.g., Nekrasov, “Plant genome editing made easy: targeted mutagenesis in model and crop plants using the CRISPR-Cas system,” Plant Methods 2013, 9:39 (doi:10.1186/1746-4811-9-39); Brooks, “Efficient gene editing in tomato in the first generation using the CRISPR-Cas9 system,” Plant Physiology September 2014 pp 114.247577; Shan, “Targeted genome modification of crop plants using a CRISPR-Cas system,” Nature Biotechnology 31, 686-688 (2013); Feng, “Efficient genome editing in plants using a CRISPR/Cas system,” Cell Research (2013) 23:1229-1232. doi:10.1038/cr.2013.114; published online 20 Aug. 2013; Xie, “RNA-guided genome editing in plants using a CRISPR-Cas system,” Mol Plant. 2013 November; 6(6):1975-83. doi: 10.1093/mp/sstl19. Epub 2013 Aug. 17; Xu, “Gene targeting using the Agrobacterium tumefaciens-mediated CRISPR-Cas system in rice,” Rice 2014, 7:5 (2014), Zhou et al., “Exploiting SNPs for biallelic CRISPR mutations in the outcrossing woody perennial Populus reveals 4-coumarate: CoA ligase specificity and Redundancy,” New Phytologist (2015) (Forum) 1-4 (available online only at www.newphytologist.com); Caliando et al, “Targeted DNA degradation using a CRISPR device stably carried in the host genome, NATURE COMMUNICATIONS 6:6989, DOI: 10.1038/ncomms7989, www.nature.com/naturecommunications DOI: 10.1038/ncomms7989; U.S. Pat. No. 6,603,061 —Agrobacterium-Mediated Plant Transformation Method; U.S. Pat. No. 7,868,149—Plant Genome Sequences and Uses Thereof and US 2009/0100536—Transgenic Plants with Enhanced Agronomic Traits, Morrell et al “Crop genomics: advances and applications,” Nat Rev Genet. 2011 Dec. 29; 13(2):85-96, all the contents and disclosure of each of which are herein incorporated by reference in their entirety. Aspects of utilizing the compositions, systems, and methods may be analogous to the use of the composition in plants, and mention is made of the University of Arizona website “CRISPR-PLANT” (genome.arizona.edu/crispr/) (supported by Penn State and AGI).

The compositions, systems, and methods may also be used on protoplasts. A “protoplast” refers to a plant cell that has had its protective cell wall completely or partially removed using, for example, mechanical or enzymatic means resulting in an intact biochemical competent unit of living plant that can reform their cell wall, proliferate and regenerate grow into a whole plant under proper growing conditions.

The compositions, systems, and methods may be used for screening genes (e.g., endogenous, mutations) of interest. In some examples, genes of interest include those encoding enzymes involved in the production of a component of added nutritional value or generally genes affecting agronomic traits of interest, across species, phyla, and plant kingdom. By selectively targeting e.g., genes encoding enzymes of metabolic pathways, the genes responsible for certain nutritional aspects of a plant can be identified. Similarly, by selectively targeting genes which may affect a desirable agronomic trait, the relevant genes can be identified. Accordingly, the present disclosure encompasses screening methods for genes encoding enzymes involved in the production of compounds with a particular nutritional value and/or agronomic traits.

It is also understood that reference herein to animal cells may also apply, mutatis mutandis, to plant or fungal cells unless otherwise apparent; and the enzymes herein having reduced off-target effects and systems employing such enzymes can be used in plant applications, including those mentioned herein.

In some cases, nucleic acids introduced to plants and fungi may be codon optimized for expression in the plants and fungi. Methods of codon optimization include those described in Kwon K C, et al., Codon Optimization to Enhance Expression Yields Insights into Chloroplast Translation, Plant Physiol. 2016 September; 172(1):62-77.

In example embodiments, compositions, systems, and methods herein can be used for an engineered or otherwise modified microbiome. Plant associated microbes (e.g., phytomicrobiomes) are engineered to enhance plant growth-promoting traits, such as yield or resilience. See e.g., Ke, J.; Wang, B.; Yoshikuni, Y. Microbiome Engineering: Synthetic Biology of Plant-Associated Microbiomes in Sustainable Agriculture. Trends in Biotechnology, 2021, 39, 244-261, Arif, I.; Batool, M.; Schenk, P. M. Plant Microbiome Engineering: Expected Benefits for Improved Crop Growth and Resilience. Trends in Biotechnology, 2020, 38, 1385-1396, Foo, J. L.; et al. Microbiome Engineering: Current Applications and Its Future. Biotechnology Journal, 2017, 12, 1600099, and Bano, S.; WU, X.; Zhang, X. Towards Sustainable Agriculture: Rhizosphere Microbiome Engineering. Applied Microbiology and Biotechnology, 2021, 105, 7141-7160.

In example embodiments, compositions, systems, and methods herein can be used for modifying food products. For example, cultivated meat is produced in vitro and the cells sourced for this process is an important aspect. Therefore, authenticating cell lines with modifications can add a level of safety and security to the process. See e.g., Reiss, J.; Robertson, S.; Suzuki, M. Cell Sources for Cultivated Meat: Applications and Considerations throughout the Production Workflow. International Journal of Molecular Sciences, 2021, 22, 7513 and Pajcin, I.; et al. Bioengineering Outlook on Cultivated Meat Production. Micromachines, 2022, 13, 402.

In example embodiments, compositions, systems, and methods herein can be used for modifications in agricultural-based cell bioreactors. These cell bioreactors may be used to create industrial chemicals such as fuels, in the food industry such as brewing, and cosmetics. See e.g., Eibl, R.; et al. Plant Cell Culture Technology in the Cosmetics and Food Industries: Current State and Future Trends. Applied Microbiology and Biotechnology, 2018, 102, 8661-8675.

### Examples of Plants

The compositions, systems, and methods herein can be used for modifications in essentially any plant. A wide variety of plants and plant cell systems may encrypt information. In general, the term “plant” relates to any various photosynthetic, eukaryotic, unicellular or multicellular organisms of the kingdom Plantae characteristically growing by cell division, containing chloroplasts, and having cell walls comprising of cellulose. The term plant encompasses monocotyledonous and dicotyledonous plants.

The compositions, systems, and methods may be used over a broad range of plants, such as for example with dicotyledonous plants belonging to the orders Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales; monocotyledonous plants such as those belonging to the orders Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales, or with plants belonging to Gymnospermae, e.g., those belonging to the orders Pinales, Ginkgoales, Cycadales, Araucariales, Cupressales and Gnetales.

The compositions, systems, and methods herein can be used over a broad range of plant species, included in the non-limitative list of dicot, monocot or gymnosperm genera hereunder: Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Trifolium, Trigonella, Vicia, Vinca, Vilis, and Vigna; and the genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, Zea, Abies, Cunninghamia, Ephedra, Picea, Pinus, and Pseudotsuga.

In one embodiment, target plants and plant cells for engineering include those monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). Specifically, the plants are intended to comprise without limitation angiosperm and gymnosperm plants such as acacia, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, and zucchini.

The term plant also encompasses Algae, which are mainly photoautotrophs unified primarily by their lack of roots, leaves and other organs that characterize higher plants. The compositions, systems, and methods can be used over a broad range of “algae” or “algae cells.” Examples of algae include eukaryotic phyla, including the Rhodophyta (red algae), Chlorophyta (green algae), Phaeophyta (brown algae), Bacillariophyta (diatoms), Eustigmatophyta and dinoflagellates as well as the prokaryotic phylum Cyanobacteria (blue-green algae). Examples of algae species include those of Amphora, Anabaena, Anikstrodesmis, Botryococcus, Chaetoceros, Chlamydomonas, Chlorella, Chlorococcum, Cyclotella, Cylindrotheca, Dunaliella, Emiliana, Euglena, Hematococcus, Isochrysis, Monochrysis, Monoraphidium, Nannochloris, Nannnochloropsis, Navicula, Nephrochloris, Nephroselmis, Nitzschia, Nodularia, Nostoc, Oochromonas, Oocystis, Oscillartoria, Pavlova, Phaeodactylum, Playtmonas, Pleurochrysis, Porhyra, Pseudoanabaena, Pyramimonas, Stichococcus, Synechococcus, Synechocystis, Tetraselmis, Thalassiosira, and Trichodesmium.

### Specific Plant Organelles

The compositions and systems herein may comprise modifying the genome, or a portion thereof, in a specific plant organelle. In an example embodiment, it is envisaged that the compositions and systems are used to specifically modifying chloroplast genes, or a portion thereof.

Plants with Desired Traits

The compositions, systems, and methods herein may be used to encrypt information into the genome, or a portion thereof, into plants with desired traits. This approach allows monitoring of the plants with desired traits. Monitoring may include identifying genetic alterations to the plant with desired traits or ownership of a plant with desired traits.

### Encryption of Polyploid Plants

The compositions, systems, and methods may be used to modify the genome, or a portion thereof, of polyploid plants. Polyploid plants carry duplicate copies of their genomes (e.g., as many as six, such as in wheat). In some cases, the compositions, systems, and methods may be/can be multiplexed to affect all copies of a gene, or to target dozens of genes at once. For instance, the compositions, systems, and methods may be used to simultaneously ensure encryption in different genes responsible for suppressing defenses against a disease. The modification may be simultaneous suppression the expression of the TaMLO-Al, TaMLO-Bl and TaMLO-Dl nucleic acid sequence in a wheat plant cell and regenerating a wheat plant therefrom, in order to ensure that the wheat plant is resistant to powdery mildew (e.g., as described in WO 2015/109752).

### Plant Cultures and Regeneration

In one embodiment, the modified plants or plant cells may be cultured to regenerate a whole plant which possesses modified genome information. Examples of regeneration techniques include those relying on manipulation of certain phytohormones in a tissue culture growth medium, relying on a biocide and/or herbicide marker which has been introduced together with the desired nucleotide sequences, obtaining from cultured protoplasts, plant callus, explants, organs, pollens, embryos or parts thereof.

### Detecting Modifications in the Plant Genome-Selectable Markers

When the compositions, systems, and methods are used to modify the genome, or a portion thereof, of a plant, suitable methods may be used to confirm and detect the modification made in the plant. In some examples, when a variety of modifications are made, one or more desired modifications or traits resulting from the modifications may be selected and detected. The detection and confirmation may be performed by biochemical and molecular biology techniques such as those described herein.

Modifications may be used for selecting, monitoring, isolating cells and plants with desired modifications and traits. Modifications can confer positive or negative selection and is conditional or non-conditional on the presence of external substrates.

### Applications in Fungi

The compositions, systems, and methods described herein can be used to modify the genome, or a portion thereof, in fungi or fungal cells, such as yeast. The approaches and applications in plants may be applied to fungi as well.

A fungal cell may be any type of eukaryotic cell within the kingdom of fungi, such as phyla of Ascomycota, Basidiomycota, Blastocladiomycota, Chytridiomycota, Glomeromycota, Microsporidia, and Neocallimastigomycota. Examples of fungi or fungal cells include yeasts, molds, and filamentous fungi.

In one embodiment, the fungal cell is a yeast cell. A yeast cell refers to any fungal cell within the phyla Ascomycota and Basidiomycota. Examples of yeasts include budding yeast, fission yeast, and mold, S. cerervisiae, Kluyveromyces marxianus, Issatchenkia orientalis, Candida spp. (e.g., Candida albicans), Yarrowia spp. (e.g., Yarrowia lipolytica), Pichia spp. (e.g., Pichia pastoris), Kluyveromyces spp. (e.g., Kluyveromyces lactis and Kluyveromyces marxianus), Neurospora spp. (e.g., Neurospora crassa), Fusarium spp. (e.g., Fusarium oxysporum), and Issatchenkia spp. (e.g., Issatchenkia orientalis, Pichia kudriavzevii and Candida acidothermophilum).

In one embodiment, the fungal cell is a filamentous fungal cell, which grow in filaments, e.g., hyphae or mycelia. Examples of filamentous fungal cells include Aspergillus spp. (e.g., Aspergillus niger), Trichoderma spp. (e.g., Trichoderma reesei), Rhizopus spp. (e.g., Rhizopus oryzae), and Mortierella spp. (e.g., Mortierella isabellina).

In one embodiment, the fungal cell is of an industrial strain. Industrial strains include any strain of fungal cell used in or isolated from an industrial process, e.g., production of a product on a commercial or industrial scale. Industrial strain may refer to a fungal species that is typically used in an industrial process, or it may refer to an isolate of a fungal species that may be also used for non-industrial purposes (e.g., laboratory research). Examples of industrial processes include fermentation (e.g., in production of food or beverage products), distillation, biofuel production, production of a compound, and production of a polypeptide. Examples of industrial strains include, without limitation, JAY270 and ATCC4124.

In one embodiment, the fungal cell is a polyploid cell whose genome is present in more than one copy. Polyploid cells include cells naturally found in a polyploid state, and cells that has been induced to exist in a polyploid state (e.g., through specific regulation, alteration, inactivation, activation, or modification of meiosis, cytokinesis, or DNA replication). A polyploid cell may be a cell whose entire genome is polyploid, or a cell that is polyploid in a particular genomic locus of interest. In some examples, the abundance of guide RNA may more often be a rate-limiting component in genome engineering of polyploid cells than in haploid cells, and thus the methods using the composition described herein may take advantage of using certain fungal cell types.

In one embodiment, the fungal cell is a diploid cell, whose genome is present in two copies. Diploid cells include cells naturally found in a diploid state, and cells that have been induced to exist in a diploid state (e.g., through specific regulation, alteration, inactivation, activation, or modification of meiosis, cytokinesis, or DNA replication). A diploid cell may refer to a cell whose entire genome is diploid, or it may refer to a cell that is diploid in a particular genomic locus of interest.

In one embodiment, the fungal cell is a haploid cell, whose genome is present in one copy. Haploid cells include cells naturally found in a haploid state, or cells that have been induced to exist in a haploid state (e.g., through specific regulation, alteration, inactivation, activation, or modification of meiosis, cytokinesis, or DNA replication). A haploid cell may refer to a cell whose entire genome is haploid, or it may refer to a cell that is haploid in a particular genomic locus of interest.

### Applications in Non-Human Animals

The compositions, systems, and methods may be used to authenticate/monitor non-human animals. In one embodiment, the compositions, systems, and methods may be used to improve breeding and introducing desired traits, e.g., increasing the frequency of trait-associated alleles, introgression of alleles from other breeds/species without linkage drag, and creation of de novo favorable alleles. Genes and other genetic elements that can be targeted may be screened and identified. Applications described in other sections such as therapeutic, diagnostic, etc. can also be used on the animals herein.

The compositions, systems, and methods may be used on animals such as fish, amphibians, reptiles, mammals, and birds. The animals may be farm and agriculture animals, or pets. Examples of farm and agriculture animals include, but are not limited to, horses, goats, sheep, swine, cattle, llamas, alpacas, and birds, e.g., chickens, turkeys, ducks, and geese. The animals may be non-human primates, including but not limited to, baboons, capuchin monkeys, chimpanzees, lemurs, macaques, marmosets, tamarins, spider monkeys, squirrel monkeys, and vervet monkeys. Examples of pets include, but are not limited to, dogs, cats, horses, wolves, rabbits, ferrets, gerbils, hamsters, chinchillas, fancy rats, guinea pigs, canaries, parakeets, and parrots.

### Modified Cells

In one aspect, embodiments disclosed herein are directed to biological materials that have been modified as disclosed herein. In example embodiments, a biological material is a modified organism or a modified cell. In example embodiments, the modified cells are from a prokaryote, an eukaryote, or a combination thereof. In example embodiments, the cell is modified to encode information as described herein. For example, the modified cells can include any cell line or primary cell, such as HEK293T cells. The cell(s) may comprise a cell from or in a model non-human organism, for example a model non-human mammal that comprise encrypted genomes encoding information.

In one aspect, an engineered, non-naturally occurring cell, or progeny thereof, wherein the genome of the cell is modified to store encoded information encrypted according to the methods and systems described herein. The modified cells may be generated using the gene editing systems described herein. In example embodiments, the modified cell is a therapeutic cell. Clinical application of CRISPR-Cas9 gene-edited T cells is generally safe and feasible (see, e.g., Lu Y, Xue J, Deng T, et al. Safety and feasibility of CRISPR-edited T cells in patients with refractory non-small-cell lung cancer [published correction appears in Nat Med. 2020 July; 26(7):1149]. Nat Med. 2020; 26(5):732-740; Lacey SF, Fraietta JA. First Trial of CRISPR-Edited T cells in Lung Cancer. Trends Mol Med. 2020; 26(8):713-715; and Zhang X, Cheng C, Sun W, Wang H. Engineering T Cells Using CRISPR/Cas9 for Cancer Therapy. Methods Mol Biol. 2020; 2115:419-433). Immune cells can also be edited ex vivo using Zn Finger proteins (see, e.g., Perez EE, Wang J, Miller J C, et al. Establishment of HIV-1 resistance in CD4+ T cells by genome editing using zinc-finger nucleases. Nat Biotechnol. 2008; 26(7):808-816).

To deliver nucleic acid modifying agents to cells for modification, a wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432). Viral vectors may for example include vectors based on HIV, SV40, EBV, HSV or BPV. Despite success with lentiviral delivery, Hendel et al, (Nature Biotechnology 33, 985-989 (2015) doi:10.1038/nbt.3290) showed the efficiency of editing human T-cells with chemically modified RNA, and direct RNA delivery to T-cells via electroporation.

Further embodiments are illustrated in the following Examples which are given for illustrative purposes only and are not intended to limit the scope of the invention.

### EXAMPLES

### Example 1—DNA Cryptography in Living Cells Through Multi-Site Base Editing

Here, Applicants present a workflow for robust base editing at a high number of genomic sites in mammalian cells, across cell populations, and in individual stem cells. With the increasing interest in DNA as a medium for information transfer and storage, Applicants demonstrate editing capabilities by encoding short messages across >100 genomic sites and introduce a cryptographic encoding scheme, termed Genomic Sequence Encryption (GSE). GSE addresses the challenge of ensuring information confidentiality and integrity in biological substrates and is the first cryptographic scheme based on DNA sequencing properties, utilizing the difficulty of detecting point mutations. Applicants demonstrate that the encryption scheme is impossible to break until a certain number of messages have been observed, after which there is an asymmetric difficulty in decoding messages between someone with and without a cryptographic key. Applicants outline an application for cellular signatures that can be applied to authenticate cells or supply chains. Applicants conceptualize and demonstrate an authentication scheme that enables the detection of further genetic manipulation. Notably, Applicants also demonstrate that individual stem cells with more than two dozen edits across a single genome can be obtained with minimal screening, which Applicants anticipate to the applicability of the encrypted signatures to living animals.

### Main

DNA is the fundamental information storage medium of life. In recent years, DNA has gained increased interest as a repurposed medium for encoding data without any cellular function. The same qualities that underpin DNA's success in its biological context, such as its durability and ease of copying, contribute to its attractiveness as a general medium for information transfer and storage. Following initial successful in vitro demonstrations, the concept of information encoding in DNA has been extended to living systems. Encoding information in the genome of living organisms enables information propagation over generations. For example, scientists have utilized biological systems carrying DNA barcodes to create biological ‘tags’ that enable supply chain tracing and authentication, benefiting from the added physical protection of biological systems. Despite the increased interest in information encoding in DNA, mechanisms of guaranteeing secure information transfer in DNA remain to be addressed.

Here, Applicants developed a method to securely encode information through DNA cryptography in living cells, which Applicants term Genomic Sequence Encryption (GSE). Cryptography achieves information security by requiring a cryptographic key to decrypt information—here, the key comprises the list of genomic coordinates across which point mutations can be installed. Applicants utilized base editors, precise genome editing tools that are programmable via complementary gRNA sequences, to encode information through multiple single-nucleotide edits across the genome. Applicants demonstrated that GSE provides cryptographic security through the difficulty of detecting point mutations in large genomes. In contrast to algorithmic encryption, the protection GSE offers is not based on computational hardness and is thus not affected by increases in computing performance, nor by the emergence of quantum computers, realizing Shor's algorithm. GSE thus enables the safe encoding of information readable only by a recipient who has access to the key and secures information against falsification, which Applicants anticipate will be of relevance for the verification of cell strains and animals as genetically modified organisms are becoming more commonly used. In the same way, creators of genetically engineered organisms can use these genomic signatures to prove ownership infringement, even in cases where further genomic alterations are introduced.

First, Applicants developed a multi-site base editing workflow, which enables a DNA-based encryption scheme. Applicants introduce targeted edits using adenine base editors (ABEs) that enable adenine/thymine to guanine/cytosine (A·T→G·C) nucleotide transitions and cytosine base editors (CBEs) that enable cytosine/guanine to thymine/adenine (C·G→T·A) transitions. In contrast to traditional CRISPR/Cas9 editing, base editors do not rely on double-strand breaks and thus mitigate cell death and genotoxicity due to double strand breaks, insertions/deletions and chromosomal translocations that frequently occur when targeting more than one site simultaneously. However, obtaining reliable editing over a high number of sites remains challenging. Generating cells with multiple edits requires either sequential cycles of editing and isolating edited cells for subsequent editing rounds, or extensive screening of edited clones. Efforts to address this limitation have focused on using multi-gRNA arrays, frequently coupled with antibiotic selection; however, cloning of these constructs is prone to recombination due to the repetitive nature of gRNAs. Installing and enriching for multiple edits has proven particularly challenging in primary and stem cells, which have been of interest due to their physiological relevance such as for creating organoid models with a specified set of mutations. Edits in these cells have been limited to four to five sites, and efficient editing enrichment has been restricted by relying on phenotypic selection for each of the sites. Applicants develop a workflow that enables robust, single-step multi-site base editing using pooled editing and encode information by simultaneous editing across >100 genomic sites. Using this workflow, Applicants also demonstrated that individual stem cells with more than two dozen edits can be obtained with minimal screening.

GSE is the first DNA sequencing-based encryption scheme. Prior work on information encoding in DNA relies on security through obscurity in which a short synthesized DNA sequence is hidden in DNA. However, if the hidden information is discovered once or if the security mechanism is known, the security system is immediately broken as once the DNA is sequenced, short strings of sequence that do not map to a reference genome can be easily detected. Cryptography, on the other hand, requires a cryptographic key to decrypt information. GSE adheres to Shannon's Maxim which requires that a cryptographic system remains secure, even if everything about the system is known, except for the key. In GSE information is encoded across a set of genomic coordinates which represent the cryptographic key, and information is secured through the asymmetric difficulty of detecting edits with and without the key. The information thus remains secure even if the encryption scheme is known, as long as the key remains hidden. GSE can be broadly applied as a signature for living biological materials. This allows genetically modified strains to be cryptographically signed and authenticated over generations as genomic edits are propagated.

Second, Applicants implemented GSE and demonstrated that it can detect whether a cell strain has been modified in the interim by analyzing shifts in editing frequencies. Applicants further implemented GSE through multi-site editing across a single genome. Applicants demonstrated this in single embryonic mouse stem cells. As embryonic stem cells are commonly used for zygote injection, this step paves the way toward encrypted signatures in living animals. As genome engineering for cellular therapies and genetically modified organisms become more widespread, Applicants anticipate that encrypted genomic signatures will become important for securing and authenticating cell strains, genetically engineered animals, and supply chains.

### Results

Base editors can easily be programmed towards multiple genomic targets via gRNAs, and do not rely on DNA double-strand breaks that have been demonstrated to cause cellular toxicity when multiple edits are introduced. Applicants thus reasoned that base editors are a suitable editing tool to introduce multiple edits in a single round. Robust multi-site editing would in turn enable GSE, which comprises precise edits across distinct genomic loci. Additionally, Applicants aimed to use plasmids that each encode a single gRNA, thus enabling Applicants to easily combine different subsets of gRNAs for encoding information (FIG. 1a).

Applicants first characterized how the number of distinct gRNA plasmids impacted the editing efficiency per site. Applicants assessed pool sizes of up to 80 gRNAs while keeping the mass of total gRNA constant and observed that increasing the number of gRNAs leads to a decrease in the relative editing efficiency per site (FIG. 1b). This relationship was observed for all tested pool sizes, except for the smallest two, four and eight gRNAs, which show only a minimal difference in editing efficiency. This may be because at large gRNA pool sizes, the editing efficiency was limited by the availability of base editors (BE) protein molecules to form BE-gRNA complexes.

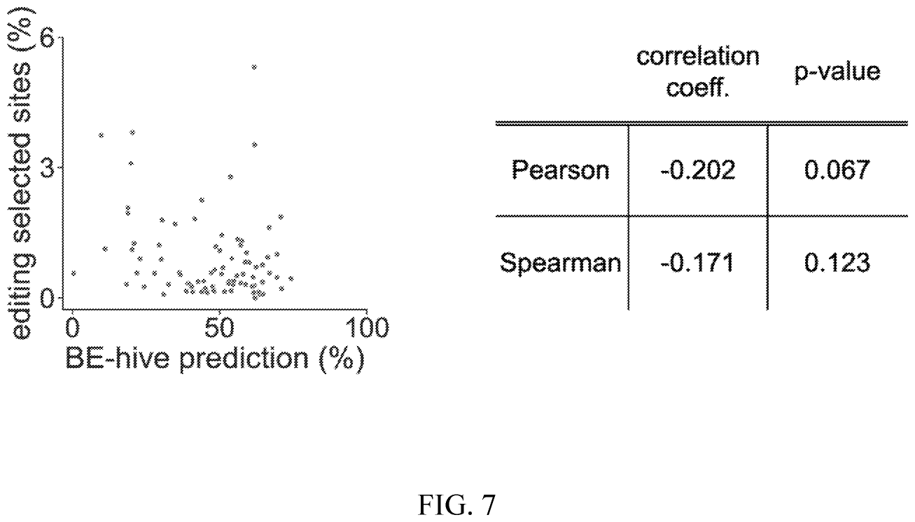

Next, Applicants evaluated gRNA editing efficiency and established an easy pooled assay to evaluate gRNA editing at endogenous genomic sites. Applicants demonstrated that editing rates of the pooled assay are strongly correlated with those of individually purified gRNAs (r=0.768; FIG. 1c, FIG. 6a-b), between two independent experiments with varied gRNA pool compositions. This result indicates that pooled screening is an efficient strategy for quick gRNA evaluation. Notably, prediction results from existing models for predicting gRNA efficiencies did not show a correlation with observed editing efficiencies (FIG. 7), suggesting that multi-site editing at endogenous loci is determined by factors not captured by current models (which might include pooled transfection on editing efficiencies or other cellular and site-specific factors) and that instead an experimental evaluation is needed.

To further investigate the robustness of editing efficiency, Applicants correlated two replicates and found that pooled editing rates for both CBEs and ABEs are highly correlated between two biological replicates (r=0.902 for CBE, r=0.970 for ABE) (FIG. 1d). These results support a reproducible one-step installation of multiple base edits.

Taken together, these results show that editing efficiency at individual sites depends on gRNA pool size, that gRNA efficiency can be quickly evaluated using pooled cloning—further indicating that editing remains stable across different gRNA pools—and that editing rates across sites are generally highly reproducible.

### Reliable Encoding and Detection of Edits at >100 Genomic Sites

Encoding information in a single editing step requires that edits can be not only robustly installed but also reliably decoded. Messages, that is, a specific edited state across sites, are encoded through pooled transfections using a subset of gRNAs. Here, Applicants choose a binary encoding scheme: Bits corresponding to zeros are unedited reference bases while bits corresponding to ones are C·Gs converted into T·As, or A·Ts converted into G·Cs, respectively (FIG. 2a). Applicants reasoned that in this binary implementation, around half of the sites would be edited for each full-length message and that Applicants can best analyze the fidelity of encoding and decoding messages by transfecting two even-sized, non-overlapping gRNA pools.

To analyze the fidelity with which intended edits are installed and detected by an intended recipient, Applicants determined whether Applicants can correctly identify which gRNAs have been transfected. Applicants calculated receiver operator characteristic (ROC) curves for varying editing thresholds, defining true positives as transfected gRNAs for which editing is detected upon analysis and defining true negatives as non-transfected sites for which no editing is detected. As not all transfected gRNAs might yield editing above an allele frequency threshold, and as sites for which no gRNA was transfected might have some degree of background, Applicants expect these cases to introduce classification errors, yielding false negatives (FNs) and false positives (FPs), respectively. For evaluating cytosine base editing, a total of 110 sites were evaluated. Applicants calculated the area under the ROC curve to be 0.980, indicating that targeted and untargeted sites can be separated with high accuracy (FIG. 2b). Precision-recall analysis further showed an area under the curve of 0.978, indicating that it is possible to detect edited sites with high accuracy (high precision) as well as the majority of positive results (high recall). For evaluating adenine base editing, a total of 90 gRNAs were evaluated. Applicants calculated the area under the ROC curve to be 0.969 and the area under the precision-recall curve to be 0.968 (FIG. 2c). These results demonstrate that one-pot encoding with both CBE and ABE allows an intended recipient to discern edited and unedited sites with high accuracy.

To determine an editing threshold for detecting edited sites in a population of cells, Applicants picked an editing rate that minimizes misclassifications based on the data where 50% of sites were targeted. For CBE, Applicants found that an editing threshold of 0.1% gave best results with only 4/110 false negatives and 4/110 false positives, corresponding to a true positive rate of 96.4% and a true negative rate of 96.4% (FIG. 2d). For ABE, an editing threshold of 0.145% gave best results with only 4/90 false negatives and 4/90 false positives, corresponding to a true positive rate and true negative rate of 95.6%, respectively.

These results demonstrate a base-editing protocol that can introduce edits across a hundred sites in the mammalian genome, providing a facile method for reliable information encoding and retrieval.

### Genomic Sequence Encryption Guarantees Information Security

To demonstrate that GSE provides information security, Applicants computationally simulated the difficulty of breaking the encryption. The cryptographic key in GSE comprises the genomic indices at which mutations are installed, which Applicants also refer to as ‘key sites’. Without access to the key sites, breaking the encryption entails searching over the full length of the genome. This process is prone to: i.) missing edited sites and ii.) detecting false hits due to reasons including sequencing error, single nucleotide polymorphisms (SNPs), or artifacts occurring during library preparation (FIG. 3a.). Applicants modeled the difficulty of this brute-force decryption first for a single message, then over multiple messages to finally determine the cost for breaking the encryption.

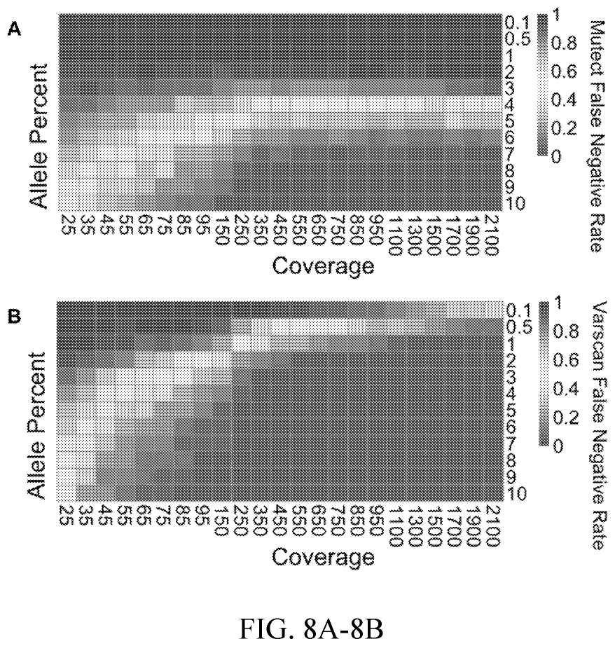

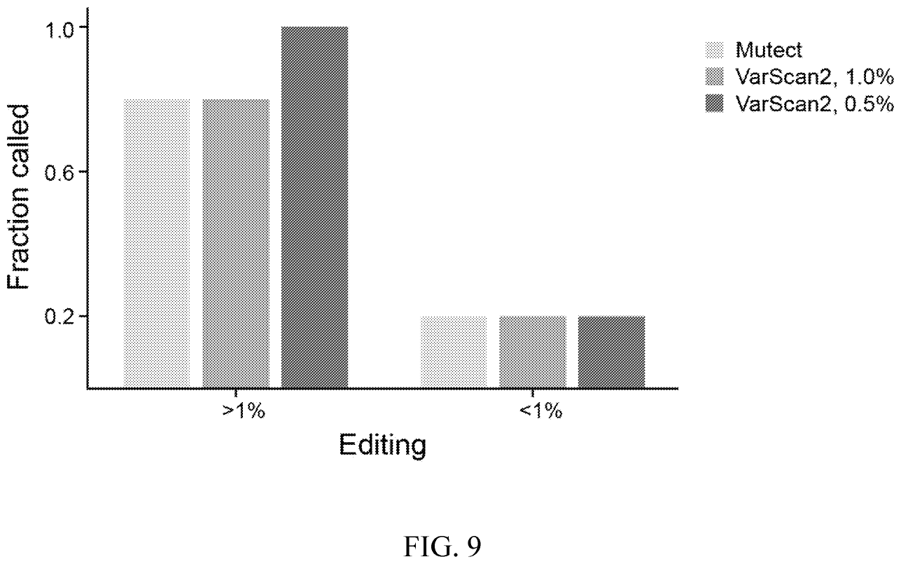

Applicants developed a simulation framework in which Applicants introduced synthetic edits into a published human deep sequencing data set, and examined the performance of commonly used variant callers (Methods). Applicants evaluated the detection of introduced mutations (false negative rate), the incorrect detection of variants (false positive rate), and their relation to the allele frequency of edits. The false negative rate was inversely proportional to both the allele frequency of the edit and sequencing coverage (FIG. 3b, FIG. 8a-b). The false positive rate depends on the variant caller sensitivity and thus indirectly on the allele frequency of the edits (FIG. 3b), as the sensitivity would need to be adjusted to allow the detection of true edits. At a sensitivity of 0.1%, the false positive rate corresponds to ~4%, or millions of sites across the human genome. These results suggest that an unintended recipient would not be able to discern index sites by observing a single message. Applicants further experimentally validated the difficulty of detecting edited sites at unknown coordinates, by inducing and analyzing silent edits across the human exome at >1000× coverage. Applicants observed both decreasing performance in detecting edits at decreasing allele frequencies (FIG. 9) and simultaneously increasing false positive rates (FIG. 10), thus further validating that even at high coverage, detecting key index sites remains challenging.

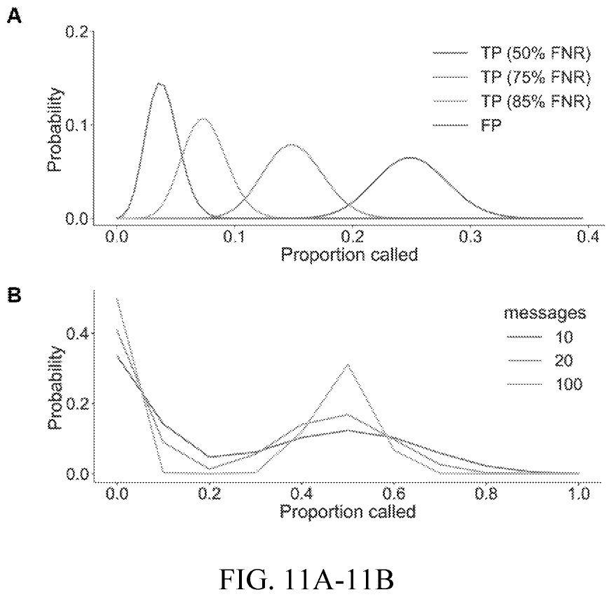

In an ideal cryptographic system, the same key could be used over multiple messages while still providing secure information transfer. Applicants hypothesized that observing multiple messages would increase the detection of both TPs and FPs due to sequencing error because the latter—in contrast to TPs or SNPs which are present in a high fraction of messages—are randomly distributed in the genome (FIG. 3c, FIG. 11a-b). A simulation of this scenario revealed that, assuming infinite sequence coverage, a minimum of 8 messages is required to break the code. With decreasing allele frequencies, the number of required messages further increases; at an editing frequency of 0.1%, an unintended recipient would need to observe at least 24 messages. (FIG. 3d). GSE is thus impossible to break and enables secure information transfer until a certain number of messages is observed, and this number depends on the allele frequency of the installed edits.

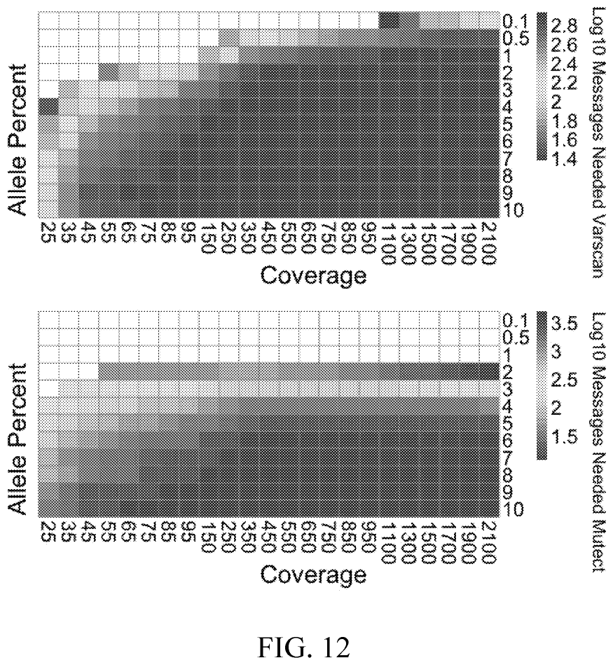

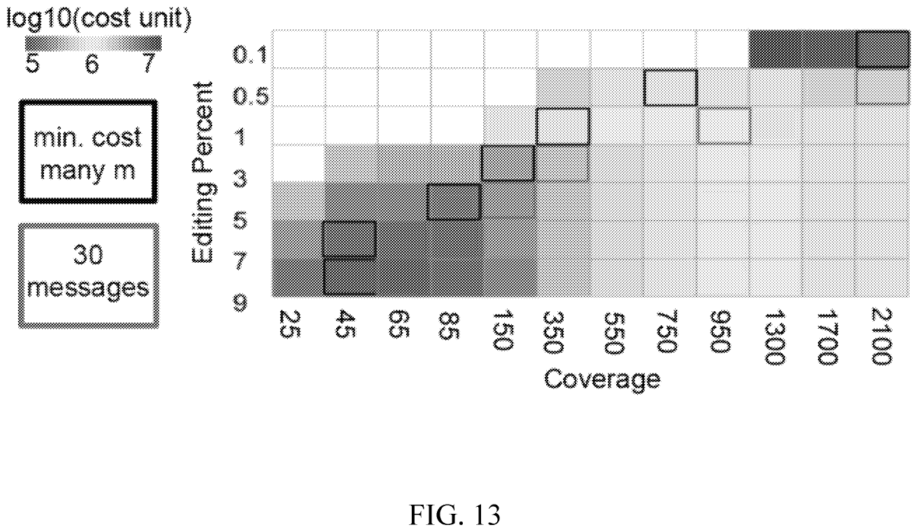

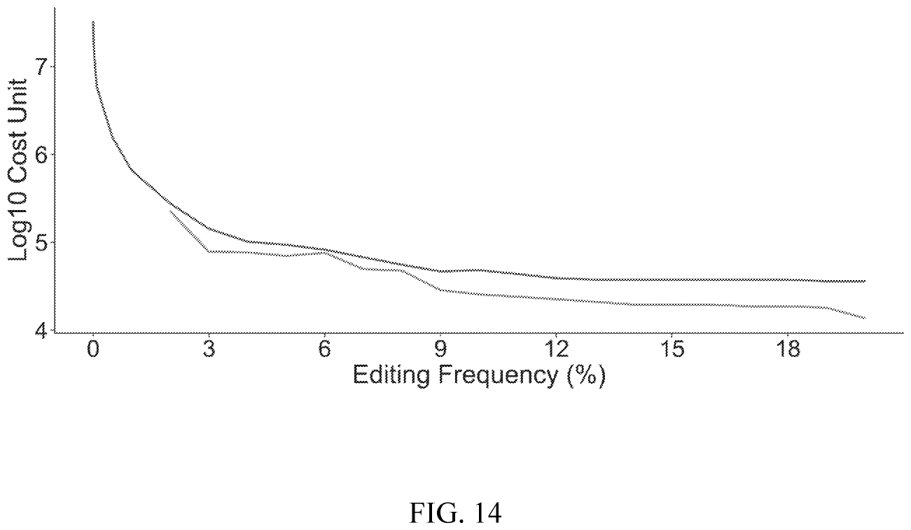

Finally, Applicants investigated the feasibility of breaking the encryption assuming that a number of messages higher than the number that provides complete security is observed. Applicants modeled this scenario under ideal conditions for the unintended recipient assuming knowledge of editing allele frequencies. Applicants calculated the total cost for two scenarios; in the first scenario an unintended recipient can freely vary sequencing coverage and the number of observed messages to achieve the lowest cost (FIG. 12), while in the second scenario, key indices need to be identified within fewer than 30 messages can be observed to break the key (FIG. 3e, FIG. 13). Compared to the cost of a recipient who has access to the key (FIG. 3f, FIG. 14), the cost of breaking the encryption is ~105 higher at allele frequencies of 5%, and this cost difference increases non-linearly at lower allele frequencies. At an editing frequency of 0.5%—which is well above the utilized detection threshold of 0.1%—the cost difference between recipients with and without the key is greater than 106.

Taken together, these results show that the message remains largely inaccessible without prior access to the key and that revealing key indices is asymmetrically difficult for an unintended recipient, requiring the observation of multiple messages even with infinite sequencing budgets. Importantly, even if the method of encoding information is known, the message remains secure, thus adhering to Shannon's maxim for secure cryptographic systems.

### Encrypted Signatures to Detect Instances of Further Genomic Manipulation

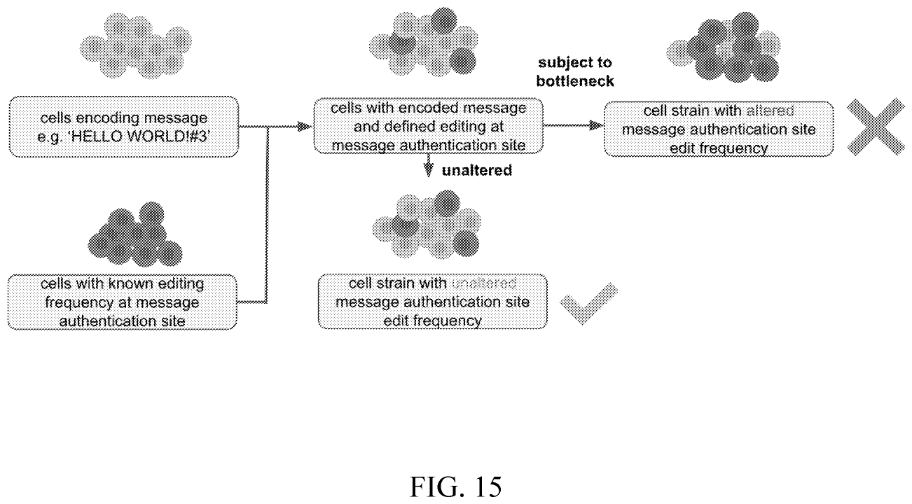

Applicants demonstrate an application of GSE for falsification-proof ‘cell signatures’, i.e. short messages in genetically modified strains that can be easily read out by an intended recipient to authenticate a cell strain. Further, Applicants show that GSE enables additionally incorporating editing at a ‘quality control’ (QC) site that allows for validating whether a cell line has been perturbed, similar to computational cryptographic schemes which frequently incorporate a method to validate the authenticity of the data. Applicants reasoned that by creating an edit at the QC site with a defined allelic frequency (FIG. 15), a recipient of a cell strain can detect whether a population of cells has been subjected to a genomic bottleneck (such as during a cell sorting or selection step upon genomic alteration), which would change the edit frequency. On the other hand, for an unmodified strain, the allelic frequency would be expected to remain stable (FIG. 3a).

First, Applicants examined changes in editing frequency when a population of cells is subjected to a genomic bottleneck compared to regular growth conditions. Applicants created a mammalian cell line with silent mutations and compared shifts in editing frequencies when cells were bottlenecked, or maintained under regular conditions (FIG. 3b). Applicants observed that edits remain more stable under regular passage conditions, where the highest absolute change in editing frequency was by a factor of 2.78 at any of the passages. In contrast, for the cell population bottlenecked at 500 cells, the highest absolute change was 5.18-fold. Applicants observed that subjecting cell populations to more stringent bottlenecks led to a larger disruption of editing frequencies (FIG. 3c). In addition, Applicants observed that 8/20 and 6/20 edited sites were no longer detected after the population was bottlenecked to 50 and 100 cells, respectively. Applicants next calculated the fraction of sites for which the editing percentage is perturbed over different log fold change thresholds (FIG. 3d). Applicants observe that the majority of sites in a 50-cell bottlenecked population are perturbed by greater than a 2-fold change in allele frequency, while none of the sites have a greater than 2-fold change in allele frequency from passaging the cells over 12 days. These results confirm differences in the shift of editing rates depending on the maintenance conditions of a cell strain. Thus, the allelic frequency at the QC site allows Applicants to gain information about whether a strain has been subjected to bottlenecks.

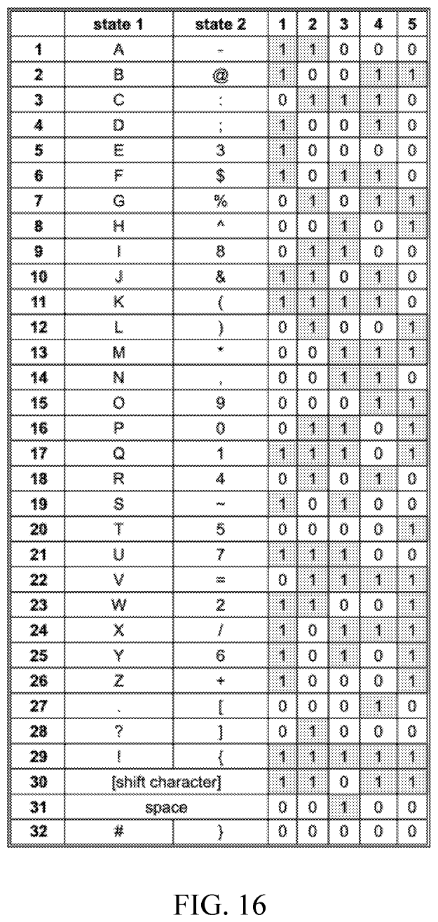

Next, Applicants demonstrated encoding short messages and installing an edit at the QC site at a desired editing frequency. Applicants employed a modified version of the five-bit International Telegraph Alphabet no. 2 (ITA2; FIG. 16) for converting text to binary. Three messages were selected for encoding, including the expected editing value at the QC site at the end of the message: ‘HELLO WORLD!#3’, ‘WHAT HATH GOD WROUGHT?’ and ‘221B BAKER STREET #2’. After transfection, an edit at the message authentication site was added at a defined editing percentage by mixing the cell population with the encoded message with a cell population that was edited only at the QC site. For each of the three messages, less than 3% of all bits were misclassified (FIG. 3e). Meanwhile, for the QC site, Applicants achieved editing values that are within ~10% of the desired editing frequencies (FIG. 3f), demonstrating that defined editing frequencies can be achieved.

In summary, these results demonstrate that even using a naive scheme with a uniform threshold for edit detection without error correction, messages can be successfully encoded and retrieved, and that GSE thus enables robust encoding and decoding of messages. Additionally, analyzing the editing rates at the QC site can be used to obtain information about whether a strain has been perturbed, such as when strains are subjected to selection steps or sorting steps when introducing genetic modifications. This is similar to message authentication concepts in cryptography—which guarantee that the message has not been modified in the interim—and thus represents a completely biological instantiation of another cryptographic key concept.

Generation of Single Embryonic Stem Cells with Multiple Edits

To extend encrypted signatures to living animals, multiple edits need to be made within a single genome. Mouse embryonic stem cells (mESCs) are routinely injected into zygotes for the generation of transgenic mice and encoding a signature in mESCs would thus extend the application of encrypted signatures to living animals (FIG. 5a) which Applicants hypothesize could then be used to generate living animals with genomic signatures.

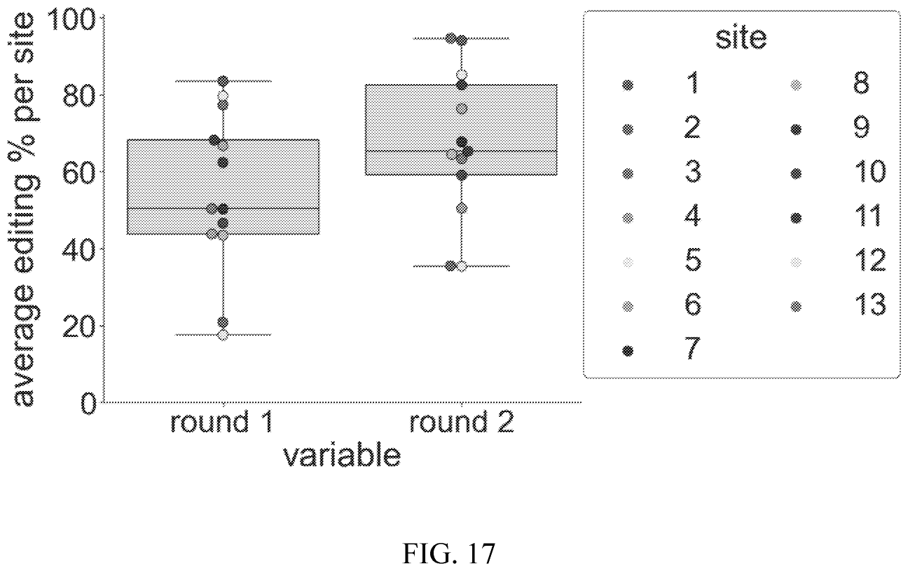

As stem cells are known to be less amenable to genome editing than differentiated cells, Applicants investigated the feasibility of simultaneously editing multiple sites in mESCs, to install a short genomic signature (“EUREKA”). Applicants observed editing across all thirteen targeted sites after one round of editing in a cell population. An iterative round of editing further increased editing rates (FIG. 17). Thus, Applicants demonstrated that Applicants could encode short genomic signatures in a population of mESCs.

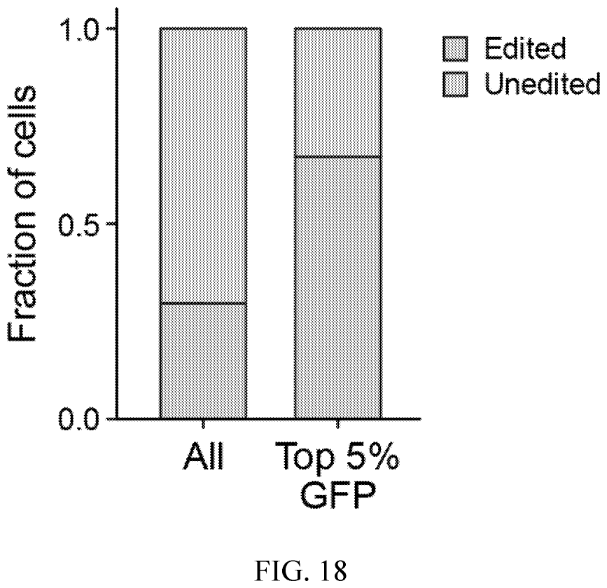

Next, Applicants determined whether individual cells carrying a mutational signature can be obtained through an enrichment workflow (FIG. 5b). As population-level editing rates do not allow Applicants to assess editing in individual cells, Applicants isolated and cultured single cells from an edited cell population. Obtaining highly edited individual cells would likely require a selection step. Applicants hypothesized that Applicants would be able to utilize co-transfection or co-editing to enrich for highly edited individual cells. First, Applicants investigated whether co-transfection with GFP can be used to enrich highly edited cells. Applicants observed an increase of editing from 35% to 67% at the site with lowest editing frequency when selecting the top 5% GFP-expressing cells (FIG. 18). This result suggests that enrichment of the top 5% GFP signal presents an easy step to increase editing.

Next, Applicants assessed whether co-editing between sites would allow one to easily find single cells with all edits. Applicants hypothesized that one could utilize the site with the lowest editing frequency to select highly edited cells. Selecting clones with editing at the lowest site, resulted in highly enriched editing and increased the average editing rate across the sites from 67 to 96% editing across all sites (allelic frequency) (FIG. 5c). This result shows that the selection of cells with editing at the site with the lowest editing rate leads to highly efficient enrichment in editing.

Applications such as encoding messages in living animals would need to take the zygosity of the edit into account, as only one copy of the DNA would be passed on to progeny. When analyzing the selected clones, 100%, or 50/50 clones, had either heterozygous or homozygous editing across all of the targeted sites, with an average number of 24.5 out of 26 edits (13 sites×2 in a diploid genome) (FIG. 5d). Each of the analyzed selected cells had at least 21 or more edits. The editing mode was 25 edits (46% of cells) with 26 edits, corresponding to homozygous editing at all targeted sites, being the second most frequent number of edits in cells (22% of cells). These results suggest that screening for editing at one of the sites allows for easy identification of multiplexed edited clones and that cells with homozygous edits at all sites can be identified with minimal screening.

Taken together, these results further demonstrate that the number of edited sites is not evenly distributed across a population of cells but that it is possible to isolate individual cells that are highly edited with minimal screening. Identifying a site with low editing efficiencies compared to other targeted sites enables a facile screening method to identify such cells. The results further demonstrate that signatures can be installed in individual mESCs, which can then be used to generate transgenic animals via zygote injections.

### DISCUSSION

Applicants developed an approach for simultaneous base editing of multiple endogenous genomic sites in mammalian cells. Applicants conceptualize GSE, the first cryptographic system based on DNA and sequencing properties. Applicants show that GSE provides information security through asymmetric sequencing costs mediated by the cryptographic key, i.e. knowledge of the genomic coordinates at which edits are installed. Applicants apply the encoding scheme for a demonstration of cell line authentication where a recipient of a cell strain could authenticate a strain by reading out the falsification-proof genomic signature. Further, Applicants propose a QC site that allows for verifying that no genomic alterations were introduced in the interim, as bottlenecks (such as during further genomic manipulation and selection of cells) would be detected. Lastly, Applicants demonstrated that the editing and encoding scheme can be implemented in individual mESCs, thus extending encrypted signatures to living animals.

Applicants show that base editors are suitable for introducing multiple simultaneous edits in mammalian cells. Introducing multiple base edits in a single genome enables the interrogation of genomic interactions at single-base resolution or at gene level through stop codon generation. Base editors have been suggested to enable a higher number of edits within the same genome, overcoming the toxicity barrier of traditional Cas912. Here, Applicants show that editing efficiency decreases per site as the number of gRNAs increases, which is likely due to a limited amount of expressed BE protein and thus BE-gRNA complexes. Previous efforts in stem cells have been limited to the introduction of four to five distinct edits simultaneously. At these edit numbers, Applicants would not yet expect a decrease in editing efficiency due to large gRNA pool sizes. Applicants demonstrated multi-site editing in mESCs, which will be of particular interest due to their high physiological relevance. Applicants demonstrated a facile enrichment protocol to obtain cells that have more than two dozen edits across a single genome. While Applicants have not yet tested the protocol for other gRNA pool sizes, Applicants anticipate that it enables the selection of cells bearing a higher number of mutations. Additionally, Applicants anticipate that coupling low-efficiency editing with a selectable phenotype, e.g. an antibiotic or fluorescent marker, will further facilitate the selection of highly edited clones.

This cryptographic system can be generalized to other organisms amenable to parallelized genome editing. GSE can be extended to include other information security concepts, such as ‘winnowing and chaffing’, where additional edits that do not include information are installed to add noise. A sparser encoding where a lower fraction of the total sites is edited, would further increase the difficulty of breaking the code. Moreover, Applicants envision that by targeting sites of common genetic variation, the message would become nearly indistinguishable from SNPs.

Applicants demonstrated the encoding of up to 110-bit messages through base editing. Applicants anticipate that longer messages can be encoded simultaneously by screening for a larger number of gRNAs with high editing efficiencies. Editing efficiency per site decreases with increasing number of gRNAs in the pools, thus there are limits to the number of edits that can be made within a single round. This challenge can be circumvented through iterative rounds of transfection. While the base editors used in this study require the presence of an NGG PAM site —thus imposing some restrictions on targeted sites—newer evolved base editors with relaxed or altered PAM site requirements could be employed to expand the accessible sequence space. More recently, the development of novel base editors that enable base transversions allow for the introduction of nearly all types of point mutations. In addition, Applicants expect that the scheme could be expanded to prime editors.

### REFERENCES FOR EXAMPLE 1

1. Goldman, N. et al. Towards practical, high-capacity, low-maintenance information storage in synthesized DNA. Nature 494, 77-80 (2013).
2. Church, G. M., Gao, Y. & Kosuri, S. Next-generation digital information storage in DNA. Science 337, 1628 (2012).
3. Shipman, S. L., Nivala, J., Macklis, J. D. & Church, G. M. CRISPR-Cas encoding of a digital movie into the genomes of a population of living bacteria. Nature 547, 345-349 (2017).
4. Yim, S. S. et al. Robust direct digital-to-biological data storage in living cells. Nat. Chem. Biol. 17, 246-253 (2021).
5. Farzadfard, F. et al. Single-Nucleotide-Resolution Computing and Memory in Living Cells. Mol. Cell 75, 769-780.e4 (2019).
6. Borkowski, O., Gilbert, C. & Ellis, T. SYNTHETIC BIOLOGY. On the record with E. coli DNA. Science vol. 353 444-445 (2016).
7. Choi, J. et al. A time-resolved, multi-symbol molecular recorder via sequential genome editing. Nature 608, 98-107 (2022).
8. Qian, J. et al. Barcoded microbial system for high-resolution object provenance. Science 368, 1135-1140 (2020).
9. Gaudelli, N. M. et al. Programmable base editing of A·T to G·C in genomic DNA without DNA cleavage. Nature 551, 464-471 (2017).
10. Komor, A. C., Kim, Y. B., Packer, M. S., Zuris, J. A. & Liu, D. R. Programmable editing of a target base in genomic DNA without double-stranded DNA cleavage. Nature 533, 420-424 (2016).
11. Martin-Rufino,J. D. et al. Massivelyparallelbaseeditingtomapvarianteffectsinhuman hematopoiesis. Cell 186, 2456-2474.e24 (2023).
12. Kuscu, C. et al. CRISPR-STOP: gene silencing through base-editing-induced nonsense mutations. Nat. Methods 14, 710-712 (2017).
13. Spencer, D. H. et al. Performance of common analysis methods for detecting low-frequency single nucleotide variants in targeted next-generation sequence data. J. Mol. Diagn. 16, 75-88 (2014).
14. Xu, C., Nezami Ranjbar, M. R., Wu, Z., DiCarlo, J. & Wang, Y. Detecting very low allele fraction variants using targeted DNA sequencing and a novel molecular barcode-aware variant caller. BMC Genomics 18, 5 (2017).
15. Shor, P. W. Algorithms for quantum computation: discrete logarithms and factoring. in Proceedings 35th Annual Symposium on Foundations of Computer Science 124-134 (1994).
16. Smith, C. J. et al. Enabling large-scale genome editing at repetitive elements by reducing DNA nicking. Nucleic Acids Res. 48, 5183-5195 (2020).
17. Fiumara, M. et al. Genotoxic effects of base and prime editing in human hematopoietic stem cells. Nat. Biotechnol. (2023) doi:10.1038/s41587-023-01915-4.
18. Aguirre, A. J. et al. Genomic Copy Number Dictates a Gene-Independent Cell Response to CRISPR/Cas9 Targeting. Cancer Discov. 6, 914-929 (2016).
19. Chen, Y. et al. Multiplex base editing to convert TAG into TAA codons in the human genome. Nat. Commun. 13, 4482 (2022).
20. Li, H. et al. Multiplex precision gene editing by a surrogate prime editor in rice. Mol. Plant 15, 1077-1080 (2022).
21. Yuan, Q. & Gao, X. Multiplex base- and prime-editing with drive-and-process CRISPR arrays. Nat. Commun. 13, 2771 (2022).
22. Katti, A. et al. Generation of precision preclinical cancer models using regulated in vivo base editing. Nat. Biotechnol. (2023) doi:10.1038/s41587-023-01900-x.
23. Geurts, M. H. et al. One-step generation of tumor models by base editor multiplexing in adult stem cell-derived organoids. Nat. Commun. 14, 4998 (2023).
24. Clelland, C. T., Risca, V. & Bancroft, C. Hiding messages in DNA microdots. Nature 399, 533-534 (1999).
25. Shannon, C. E. Communication theory of secrecy systems. The Bell System Technical Journal 28, 656-715 (1949).
26. Arbab, M. et al. Determinants of Base Editing Outcomes from Target Library Analysis and Machine Learning. Cell 182, 463-480.e30 (2020).
27. Schiroli, G. et al. Precise Gene Editing Preserves Hematopoietic Stem Cell Function following Transient p53-Mediated DNA Damage Response. Cell Stem Cell24, 551-565.e8 (2019).
28. Rivest, R. L. Chaffing and winnowing: Confidentiality without encryption. citeseerx.ist.psu.edu/viewdoc/download?doi=10.1.1.309.7743&rep=rep1&type=pdf.
29. Walton, R. T., Christie, K. A., Whittaker, M. N. & Kleinstiver, B. P. Unconstrained genome targeting with near-PAMless engineered CRISPR-Cas9 variants. Science 368, 290-296 (2020).
30. Chatterjee, P. et al. A Cas9 with PAM recognition for adenine dinucleotides. Nat. Commun. 11, 2474 (2020).
31. Kurt, I. C. et al. CRISPR C-to-G base editors for inducing targeted DNA transversions in human cells. Nat. Biotechnol. 39, 41-46 (2021).
32. Zhao, D. et al. Glycosylase base editors enable C-to-A and C-to-G base changes. Nat. Biotechnol. 39, 35-40 (2021).
33. Tong, H. et al. Programmable A-to-Y base editing by fusing an adenine base editor with an N-methylpurine DNA glycosylase. Nat. Biotechnol. 41, 1080-1084 (2023).
34. Anzalone, A. V. et al. Search-and-replace genome editing without double-strand breaks or donor DNA. Nature 576, 149-157 (2019).

Various modifications and variations of the described methods, pharmaceutical compositions, and kits will be apparent to those skilled in the art without departing from the scope and spirit of the invention. Although described in connection with specific embodiments, it will be understood that the disclosure is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A method of enriching for multi-site edited cells, comprising:
a. delivering to a population of cells, a set of guide molecules and one or more programmable nucleases, wherein the set of guide molecules directs the one or more programmable nucleases to introduce a set of edits to a plurality of genomic loci to generate an edited population of cells;
b. screening the population of cells for one or more enrichment markers; and
c. isolating one or more cells from the population of cells that comprise the one or more enrichment markers.

2. The method of claim 1, wherein the one or more enrichment markers comprise an editing efficiency, an exogenous reporter, or a combination thereof.

3. The method of claim, wherein editing efficiency is determined by sequencing one or more genomic loci having a low overall editing efficiency in the plurality of genomic loci, wherein an edit above an editing efficiency threshold at the one or more genomic loci is an indicator that a cell comprises all edits in the set of edits.

4. The method of claim, wherein the one or more genomic loci having a low overall editing efficiency are associated with a functional phenotype or an encoded reporter gene, wherein the functional phenotype or encoded reported gene are expressed if the one or more genomic loci having the low overall editing efficiency are successfully edited.

5. The method of claim, wherein the functional phenotype is antibiotic resistance.

6. The method of claim, wherein the editing efficiency threshold is at least 0.1%, 0.2%, 0.5%, or 1%.

7. The method of claim 2, wherein the exogenous reporter is co-delivered to the population of cells.

8. The method of claim, wherein the exogenous reporter is a fluorescent protein.

9. The method of claim, wherein one or more cells that express the exogenous reporter above an expression threshold are isolated.

10. The method of claim, wherein the expression threshold is in a top 20%, 15%, 10%, 5%, or 1% of the edited population of cells.

11. The method of claim 1, wherein the method further comprises, before step (a), preparing a screening pool of guide molecule vectors comprising the set of guide molecules, wherein the set of guide molecules target a plurality of target sites in a cell genome and are configured to introduce the set of edits.

12. The method of claim 1, wherein step (b) is conducted in replicate and selecting the one or more cells comprises selecting one or more cells with a correlated enrichment.

13. The method of claim 1, wherein the one or more programmable nucleases is a base editor comprising a nucleotide deaminase, a prime editor, or a Cas or OMEGA nuclease.

14. The method of claim, wherein the nucleotide deaminase is a cytidine deaminase or an adenosine deaminase.

15. (canceled)

16. (canceled)

17. An engineered cell comprising edits at a plurality of genomic loci and obtained by the method of claim 1.

18. The engineered cell of claim, wherein the engineered cell is a primary cell or a stem cell.

19. The engineered cell of claim 17, wherein the edits at the plurality of genomic loci are cryptographically encoded information.

20. The engineered cell of claim, wherein the engineered cell comprises one or more additional engineered modifications and wherein the cryptographically encoded information identifies the one or more additional engineered modifications.

21. The engineered cell of claim 17, wherein the engineered cell is an adoptive cell therapeutic.

22. The engineered cell of claim 17, wherein the engineered cell is a cell factory.
